SEQUENCE LISTING (17 sequences)

SEQ ID NO: 1 - DNA, 2030 bp, Ebola virus
  source 1..2030: mol_type = genomic DNA
atgggtgttacaggaatattgcagttacctcgtgatcgattcaagaggacatcattctttctttgggtaattatccttttccaaagaacattttccatcccgcttggagttatccacaatagtacattacaggttagtgatgtcgacaaactagtttgtcgtgacaaactgtcatccacaaatcaattgagatcagttggactgaatctcgaggggaatggagtggcaactgacgtgccatctgtgactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccatgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtcgagtggctattattctaccacaattagatatcaggctaccggttttggaactaatgagacagagtacttgttcgaggttgacaatttgacctacgtccaacttgaatcaagattcacaccacagtttctgctccagctgaatgagacaatatatgcaagtgggaagaggagcaacaccacgggaaaactaatttggaaggtcaaccccgaaattgatacaacaatcggggagtgggccttctgggaaactaaaaaaacctcactagaaaaattcgcagtgaagagttgtctttcacagctgtatcaaacggacccaaaaacatcagtggtcagagtccggcgcgaacttcttccgacccagagaccaacacaacaaatgaagaccacaaaatcatggcttcagaaaattcctctgcaatggttcaagtgcacagtcaaggaaggaaagctgcagtgtcgcatctgacaacccttgccacaatctccacgagtcctcaacctcccacaaccaaaacaggtccggacaacagcacccataatacacccgtgtataaacttgacatctctgaggcaactcaagttggacaacatcaccgtagagcagacaacgacagcacagcctccgacactccccccgccacgaccgcagccggacccttaaaagcagagaacaccaacacgagtaagagcgctgactccctggacctcgccaccacgacaagcccccaaaactacagcgagactgctggcaacaacaacactcatcaccaagataccggagaagagagtgccagcagcgggaagctaggcttaattaccaatactattgctggagtagcaggactgatcacaggcgggagaaggactcgaagagaagtaattgtcaatgctcaacccaaatgcaaccccaatttacattactggactactcaggatgaaggtgctgcaatcggattggcctggataccatatttcgggccagcagccgaaggaatttacacagaggggctaatgcacaaccaagatggtttaatctgtgggttgaggcagctggccaacgaaacgactcaagctctccaactgttcctgagagccacaactgagctgcgaaccttttcaatcctcaaccgtaaggcaattgacttcctgctgcagcgatggggtggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggtgttataattgcagttatcgctttattctgtatatgcaaatttgtcttttag

SEQ ID NO: 2 - DNA, 2031 bp, Ebola virus
  misc_feature 880: note = Addition of A nucleotide
  source 1..2031: mol_type = other DNA
atgggtgttacaggaatattgcagttacctcgtgatcgattcaagaggacatcattctttctttgggtaattatccttttccaaagaacattttccatcccgcttggagttatccacaatagtacattacaggttagtgatgtcgacaaactagtttgtcgtgacaaactgtcatccacaaatcaattgagatcagttggactgaatctcgaggggaatggagtggcaactgacgtgccatctgtgactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccatgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtcgagtggctattattctaccacaattagatatcaggctaccggttttggaactaatgagacagagtacttgttcgaggttgacaatttgacctacgtccaacttgaatcaagattcacaccacagtttctgctccagctgaatgagacaatatatgcaagtgggaagaggagcaacaccacgggaaaactaatttggaaggtcaaccccgaaattgatacaacaatcggggagtgggccttctgggaaactaaaaaaaacctcactagaaaaattcgcagtgaagagttgtctttcacagctgtatcaaacggacccaaaaacatcagtggtcagagtccggcgcgaacttcttccgacccagagaccaacacaacaaatgaagaccacaaaatcatggcttcagaaaattcctctgcaatggttcaagtgcacagtcaaggaaggaaagctgcagtgtcgcatctgacaacccttgccacaatctccacgagtcctcaacctcccacaaccaaaacaggtccggacaacagcacccataatacacccgtgtataaacttgacatctctgaggcaactcaagttggacaacatcaccgtagagcagacaacgacagcacagcctccgacactccccccgccacgaccgcagccggacccttaaaagcagagaacaccaacacgagtaagagcgctgactccctggacctcgccaccacgacaagcccccaaaactacagcgagactgctggcaacaacaacactcatcaccaagataccggagaagagagtgccagcagcgggaagctaggcttaattaccaatactattgctggagtagcaggactgatcacaggcgggagaaggactcgaagagaagtaattgtcaatgctcaacccaaatgcaaccccaatttacattactggactactcaggatgaaggtgctgcaatcggattggcctggataccatatttcgggccagcagccgaaggaatttacacagaggggctaatgcacaaccaagatggtttaatctgtgggttgaggcagctggccaacgaaacgactcaagctctccaactgttcctgagagccacaactgagctgcgaaccttttcaatcctcaaccgtaaggcaattgacttcctgctgcagcgatggggtggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggtgttataattgcagttatcgctttattctgtatatgcaaatttgtcttttag

SEQ ID NO: 3 - protein, 676 aa, Ebola virus
MGVTGILQLPRDRFKRTSFFLWVIILFQRTFSIPLGVIHNSTLQVSDVDKLVCRDKLSSTNQLRSVGLNLEGNGVATDVPSVTKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQPPTTKTGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 4 - DNA, 2031 bp, Ebola virus
  source 1..2031: mol_type = other DNA
atgggagtaactggaattctacaactaccaagagatagattcaaaagaacatctttttttctatgggttataattctatttcaaagaacattttctattccattgggagtaattcataattctacattgcaagtatctgatgtagataaactagtatgtagagataaattgtctagtacaaatcaactaagatctgtaggattgaatctagaaggaaatggtgtagcgacagatgttccatctgtaacaaaaagatggggttttagatctggtgtaccaccaaaagtagtaaattatgaagcgggagaatgggcggaaaattgttataatctagaaattaaaaaaccagatggatctgaatgtctaccagcggcgccagatggaattagaggatttccaagatgtagatatgttcataaagtatctggaacaggaccatgtgcgggagattttgcgtttcataaagaaggagcattttttctatatgatagactagcgtctacagtaatatatagaggaacaacatttgcggaaggtgtagtagcttttctaattctaccacaagcgaaaaaagatttttttagttctcatccactaagagaaccagtaaatgcgacagaagatccttcttctggatattattctactacaattagatatcaagcgacaggatttggaacaaatgaaacagaatatctatttgaagttgataatctaacatatgtacaactagaaagtagattcacaccacaatttctattgcaattgaatgaaacaatatatgcgtctggaaaaagatctaatacaactggaaaactaatttggaaagtaaatccagaaattgatacaacaattggagaatgggctttttgggaaacaaaaaaaaatttgacaagaaaaattagatctgaagaattgtcttttacagcggtatctaatggaccaaaaaatatttctggacaatctccagcgagaacttcttctgatccagaaacaaatactacaaatgaagatcacaaaattatggcgtctgaaaattcttctgctatggtacaagtacattctcaaggaagaaaagcggcggtatctcatctaacaacactagcgactatttctacatctccacaaccaccaacaacaaaaactggaccagataatagtacacataatactccagtttataaactagatatttctgaagcgacacaagttggacaacatcatagaagagcggataatgattctacagcgtctgatacaccaccagctacaacagctgctggaccattgaaagcggaaaatacaaatacttctaaatctgcggattctctagatttggcgacaacaacttctcctcaaaattattctgaaacagcgggaaataataatactcatcatcaagatactggagaagaatctgcgtctagtggaaaattgggactaattacaaatacaattgcgggtgtagcgggattgattactggtggaagaagaactagaagagaagtaatagttaatgcgcaacctaaatgtaatccaaatctacattattggacaactcaagatgaaggtgctgcgattggactagcttggattccatattttggacctgcggcggaaggaatatatactgaaggactaatgcataatcaagatggactaatttgtggactaagacaactagcgaatgaaactacacaagcgctacaactatttttgagagcgacaacagaactaagaacttttagtattctaaatagaaaagcgattgattttttgctacaaagatggggaggaacatgtcatattctaggaccagattgttgtattgaaccacatgattggacaaaaaatattacagacaaaattgatcaaattattcatgattttgttgataaaacactaccagatcaaggagataatgataattggtggacaggatggagacaatggattccagcgggaattggagtaacaggtgtaattattgcggttattgcgctattttgtatatgtaaatttgttttttaa

SEQ ID NO: 5 - DNA, 2031 bp, Ebola virus
  source 1..2031: mol_type = other DNA
atgggagtaactggaattctacaactaccaagagatagattcaaaagaacatctttctttctatgggttataattctatttcaaagaacattttctattccattgggagtaattcataattctacattgcaagtatctgatgtagataaactagtatgtagagataaattgtctagtacaaatcaactaagatctgtaggattgaatctagaaggaaatggtgtagcgacagatgttccatctgtaacaaagagatggggttttagatctggtgtaccaccaaaagtagtaaattatgaagcgggagaatgggcggaaaattgttataatctagaaattaagaaaccagatggatctgaatgtctaccagcggcgccagatggaattagaggatttccaagatgtagatatgttcataaagtatctggaacaggaccatgtgcgggagattttgcgtttcataaagaaggagcattctttctatatgatagactagcgtctacagtaatatatagaggaacaacatttgcggaaggtgtagtagcttttctaattctaccacaagcgaagaaagatttctttagttctcatccactaagagaaccagtaaatgcgacagaagatccttcttctggatattattctactacaattagatatcaagcgacaggatttggaacaaatgaaacagaatatctatttgaagttgataatctaacatatgtacaactagaaagtagattcacaccacaatttctattgcaattgaatgaaacaatatatgcgtctggaaagagatctaatacaactggaaaactaatttggaaagtaaatccagaaattgatacaacaattggagaatgggctttctgggaaacaaagaagaatttgacaagaaagattagatctgaagaattgtcttttacagcggtatctaatggaccaaagaatatttctggacaatctccagcgagaacttcttctgatccagaaacaaatactacaaatgaagatcacaaaattatggcgtctgaaaattcttctgctatggtacaagtacattctcaaggaagaaaagcggcggtatctcatctaacaacactagcgactatttctacatctccacaaccaccaacaacaaagactggaccagataatagtacacataatactccagtttataaactagatatttctgaagcgacacaagttggacaacatcatagaagagcggataatgattctacagcgtctgatacaccaccagctacaacagctgctggaccattgaaagcggaaaatacaaatacttctaaatctgcggattctctagatttggcgacaacaacttctcctcaaaattattctgaaacagcgggaaataataatactcatcatcaagatactggagaagaatctgcgtctagtggaaaattgggactaattacaaatacaattgcgggtgtagcgggattgattactggtggaagaagaactagaagagaagtaatagttaatgcgcaacctaaatgtaatccaaatctacattattggacaactcaagatgaaggtgctgcgattggactagcttggattccatattttggacctgcggcggaaggaatatatactgaaggactaatgcataatcaagatggactaatttgtggactaagacaactagcgaatgaaactacacaagcgctacaactattcttgagagcgacaacagaactaagaacttttagtattctaaatagaaaagcgattgatttcttgctacaaagatggggaggaacatgtcatattctaggaccagattgttgtattgaaccacatgattggacaaagaatattacagacaaaattgatcaaattattcatgattttgttgataaaacactaccagatcaaggagataatgataattggtggacaggatggagacaatggattccagcgggaattggagtaacaggtgtaattattgcggttattgcgctattttgtatatgtaaatttgttttttaa

SEQ ID NO: 6 - DNA, 2041 bp, Ebola virus
  source 1..2041: mol_type = other DNA
atgggagtaactggaattctacaactaccaagagatagattcaaaagaacatctttctttctatgggttataattctatttcaaagaacattttctattccattgggagtaattcataattctacattgcaagtatctgatgtagataaactagtatgtagagataaattgtctagtacaaatcaactaagatctgtaggattgaatctagaaggaaatggtgtagcgacagatgttccatctgtaacaaagagatggggttttagatctggtgtaccaccaaaagtagtaaattatgaagcgggagaatgggcggaaaattgttataatctagaaattaagaaaccagatggatctgaatgtctaccagcggcgccagatggaattagaggatttccaagatgtagatatgttcataaagtatctggaacaggaccatgtgcgggagattttgcgtttcataaagaaggagcattctttctatatgatagactagcgtctacagtaatatatagaggaacaacatttgcggaaggtgtagtagcttttctaattctaccacaagcgaagaaagatttctttagttctcatccactaagagaaccagtaaatgcgacagaagatccttcttctggatattattctactacaattagatatcaagcgacaggatttggaacaaatgaaacagaatatctatttgaagttgataatctaacatatgtacaactagaaagtagattcacaccacaatttctattgcaattgaatgaaacaatatatgcgtctggaaagagatctaatacaactggaaaactaatttggaaagtaaatccagaaattgatacaacaattggagaatgggctttctgggaaacaaagaagaatttgacaagaaagattagatctgaagaattgtcttttacagcggtatctaatggaccaaagaatatttctggacaatctccagcgagaacttcttctgatccagaaacaaatactacaaatgaagatcacaaaattatggcgtctgaaaattcttctgctatggtacaagtacattctcaaggaagaaaagcggcggtatctcatctaacaacactagcgactatttctacatctccacaaccaccaacaacaaagactggaccagataatagtacacataatactccagtttataaactagatatttctgaagcgacacaagttggacaacatcatagaagagcggataatgattctacagcgtctgatacaccaccagctacaacagctgctggaccattgaaagcggaaaatacaaatacttctaaatctgcggattctctagatttggcgacaacaacttctcctcaaaattattctgaaacagcgggaaataataatactcatcatcaagatactggagaagaatctgcgtctagtggaaaattgggactaattacaaatacaattgcgggtgtagcgggattgattactggtggaagaagaactagaagagaagtaatagttaatgcgcaacctaaatgtaatccaaatctacattattggacaactcaagatgaaggtgctgcgattggactagcttggattccatattttggacctgcggcggaaggaatatatactgaaggactaatgcataatcaagatggactaatttgtggactaagacaactagcgaatgaaactacacaagcgctacaactattcttgagagcgacaacagaactaagaacttttagtattctaaatagaaaagcgattgatttcttgctacaaagatggggaggaacatgtcatattctaggaccagattgttgtattgaaccacatgattggacaaagaatattacagacaaaattgatcaaattattcatgattttgttgataaaacactaccagatcaaggagataatgataattggtggacaggatggagacaatggattccagcgggaattggagtaacaggtgtaattattgcggttattgcgctattttgtatatgtaaatttgttttttaataatttttat

SEQ ID NO: 7 - DNA, 981 bp, Ebola virus
  source 1..981: mol_type = genomic DNA
atgaggcgggttatattgcctactgctcctcctgaatatatggaggccatataccctgccaggtcaaattcaacaattgctaggggtggcaacagcaatacaggcttcctgacaccggagtcagtcaatggagacactccatcgaatccactcaggccaattgctgatgacaccatcgaccatgccagccacacaccaggcagtgtgtcatcagcattcatcctcgaagctatggtgaatgtcatatcgggccccaaagtgctaatgaagcaaattccaatttggcttcctctaggtgtcgctgatcaaaagacctacagctttgactcaactacggccgccatcatgcttgcttcatatactatcacccatttcggcaaggcaaccaatccgcttgtcagagtcaatcggctgggtcctggaatcccggatcaccccctcaggctcctgcgaattggaaaccaggctttcctccaggagttcgttcttccaccagtccaactaccccagtatttcacctttgatttgacagcactcaaactgatcactcaaccactgcctgctgcaacatggaccgatgacactccaactggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggtatcgaagtgccagaaactctggtccacaagctgaccggtaagaaggtgacttccaaaaatggacaaccaatcatccctgttcttttgccaaagtacattgggttggacccggtggctccaggagacctcaccatggtaatcacacaggattgtgacacgtgtcattctcctgcaagtcttccagctgtggttgagaagtaa

SEQ ID NO: 8 - DNA, 981 bp, Ebola virus
  source 1..981: mol_type = other DNA
atgagaagagtaattctaccaacagcgccaccagaatatatggaagcgatatatccagcgagatctaattctacaattgcgagaggtggaaattctaatactggatttctaacaccagaatctgtaaatggagatacaccatctaatccactaagaccaattgcggatgatacaatagatcatgcgagtcatactccaggatctgtatcttctgcttttattctagaagctatggttaatgtaatttctggaccaaaagtactaatgaaacaaattccaatttggctaccattgggagtagcggatcaaaagacatattcttttgattctactacagcggcgattatgctagcgtcttatacaattacacattttggaaaagcgacaaatccactagttagagtaaatagactaggacctggaataccagatcatccattgagactactaagaattggaaatcaagcttttctacaagaatttgttctaccaccagtacaactaccacaatactttacatttgatctaacagcgctaaaactaattacacaaccattgccagcggcgacatggacagatgatacaccaacaggatctaatggtgctctaagacctggtatttcttttcatccaaaactaagacctattctattgccaaataaatctggaaagaaaggaaattctgcggatctaacatctccagaaaagattcaagcgattatgacatctctacaagacttcaaaattgtaccaattgatccaacaaagaatattatgggaattgaagtaccagaaacactagttcataaactaactggaaagaaagtaacatctaaaaatggacaacctattattccagtattgctacctaaatatattggactagatccagtagcgcctggagatctaacaatggttattacacaagattgtgatacttgtcattctccagcgagtttgcctgcggtagtagaaaaataa

SEQ ID NO: 9 - DNA, 991 bp, Ebola virus
  source 1..991: mol_type = other DNA
atgagaagagtaattctaccaacagcgccaccagaatatatggaagcgatatatccagcgagatctaattctacaattgcgagaggtggaaattctaatactggatttctaacaccagaatctgtaaatggagatacaccatctaatccactaagaccaattgcggatgatacaatagatcatgcgagtcatactccaggatctgtatcttctgcttttattctagaagctatggttaatgtaatttctggaccaaaagtactaatgaaacaaattccaatttggctaccattgggagtagcggatcaaaagacatattcttttgattctactacagcggcgattatgctagcgtcttatacaattacacattttggaaaagcgacaaatccactagttagagtaaatagactaggacctggaataccagatcatccattgagactactaagaattggaaatcaagcttttctacaagaatttgttctaccaccagtacaactaccacaatactttacatttgatctaacagcgctaaaactaattacacaaccattgccagcggcgacatggacagatgatacaccaacaggatctaatggtgctctaagacctggtatttcttttcatccaaaactaagacctattctattgccaaataaatctggaaagaaaggaaattctgcggatctaacatctccagaaaagattcaagcgattatgacatctctacaagacttcaaaattgtaccaattgatccaacaaagaatattatgggaattgaagtaccagaaacactagttcataaactaactggaaagaaagtaacatctaaaaatggacaacctattattccagtattgctacctaaatatattggactagatccagtagcgcctggagatctaacaatggttattacacaagattgtgatacttgtcattctccagcgagtttgcctgcggtagtagaaaaataataatttttat

SEQ ID NO: 10 - DNA, 5883 bp, Ebola virus
  source 1..5883: mol_type = other DNA
gaattcggagtatacgaaccgggaaagagaagatggttaaaaataaagcgagactatttgaacgagggttccatggcagattctgccgatttagtagtactaggtgcttactatggtaaaggagcaaagggtggtatcatggcagtctttctaatgggttgttacgacgatgaatccggtaaatggaagacggttaccaagtgttcaggacacgatgataatacgttaagggagttgcaagaccaattaaagatgattaaaattaacaaggatcccaaaaaaattccagagtggttagtagttaataaaatctatattcccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatttttatggcgcgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagctccccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatttttatctcgaggccgctggtacccaacctaaaaattgaaaataaatacaaaggttcttgagggttgtgttaaattgaaagcgagaaataatcataaataagcccgggatgagaagagtaattctaccaacagcgccaccagaatatatggaagcgatatatccagcgagatctaattctacaattgcgagaggtggaaattctaatactggatttctaacaccagaatctgtaaatggagatacaccatctaatccactaagaccaattgcggatgatacaatagatcatgcgagtcatactccaggatctgtatcttctgcttttattctagaagctatggttaatgtaatttctggaccaaaagtactaatgaaacaaattccaatttggctaccattgggagtagcggatcaaaagacatattcttttgattctactacagcggcgattatgctagcgtcttatacaattacacattttggaaaagcgacaaatccactagttagagtaaatagactaggacctggaataccagatcatccattgagactactaagaattggaaatcaagcttttctacaagaatttgttctaccaccagtacaactaccacaatactttacatttgatctaacagcgctaaaactaattacacaaccattgccagcggcgacatggacagatgatacaccaacaggatctaatggtgctctaagacctggtatttcttttcatccaaaactaagacctattctattgccaaataaatctggaaagaaaggaaattctgcggatctaacatctccagaaaagattcaagcgattatgacatctctacaagacttcaaaattgtaccaattgatccaacaaagaatattatgggaattgaagtaccagaaacactagttcataaactaactggaaagaaagtaacatctaaaaatggacaacctattattccagtattgctacctaaatatattggactagatccagtagcgcctggagatctaacaatggttattacacaagattgtgatacttgtcattctccagcgagtttgcctgcggtagtagaaaaataataatttttatgtcgacctgcagctaatgtattagttaaatattaaaacttaccacgtaaaacttaaaatttaaaatgatatttcattgacagatagatcacacattatgaactttcaaggacttgtgttaactgacaattgcaaaaatcaatgggtcgttggaccattaataggaaaaggtggatttggtagtatttatactactaatgacaataattatgtagtaaaaatagagcccaaagctaacggatcattatttaccgaacaggcattttatactagagtacttaaaccatccgttatcgaagaatggaaaaaatctcacaatataaagcacgtaggtcttatcacgtgcaaggcatttggtctatacaaatccattaatgtggaatatcgattcttggtaattaatagattaggtgcagatctagatgcggtgatcagagccaataataatagattaccaaaaaggtcggtgatgttgatcggaatcgaaatcttaaataccatacaatttatgcacgagcaaggatattctcacggagatattaaagcgagtaatatagtcttggatcaaatagataagaataaattatatctagtggattacggattggtttctaaattcatgtcaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 11 - DNA, 7074 bp, Ebola virus
  source 1..7074: mol_type = other DNA
gaattccctgggacatacgtatatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataattttgagaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaggcgcgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagctcgaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaactcgaggccgctggtacccaacctaaaaattgaaaataaatacaaaggttcttgagggttgtgttaaattgaaagcgagaaataatcataaataagcccgggatgggagtaactggaattctacaactaccaagagatagattcaaaagaacatctttctttctatgggttataattctatttcaaagaacattttctattccattgggagtaattcataattctacattgcaagtatctgatgtagataaactagtatgtagagataaattgtctagtacaaatcaactaagatctgtaggattgaatctagaaggaaatggtgtagcgacagatgttccatctgtaacaaagagatggggttttagatctggtgtaccaccaaaagtagtaaattatgaagcgggagaatgggcggaaaattgttataatctagaaattaagaaaccagatggatctgaatgtctaccagcggcgccagatggaattagaggatttccaagatgtagatatgttcataaagtatctggaacaggaccatgtgcgggagattttgcgtttcataaagaaggagcattctttctatatgatagactagcgtctacagtaatatatagaggaacaacatttgcggaaggtgtagtagcttttctaattctaccacaagcgaagaaagatttctttagttctcatccactaagagaaccagtaaatgcgacagaagatccttcttctggatattattctactacaattagatatcaagcgacaggatttggaacaaatgaaacagaatatctatttgaagttgataatctaacatatgtacaactagaaagtagattcacaccacaatttctattgcaattgaatgaaacaatatatgcgtctggaaagagatctaatacaactggaaaactaatttggaaagtaaatccagaaattgatacaacaattggagaatgggctttctgggaaacaaagaagaatttgacaagaaagattagatctgaagaattgtcttttacagcggtatctaatggaccaaagaatatttctggacaatctccagcgagaacttcttctgatccagaaacaaatactacaaatgaagatcacaaaattatggcgtctgaaaattcttctgctatggtacaagtacattctcaaggaagaaaagcggcggtatctcatctaacaacactagcgactatttctacatctccacaaccaccaacaacaaagactggaccagataatagtacacataatactccagtttataaactagatatttctgaagcgacacaagttggacaacatcatagaagagcggataatgattctacagcgtctgatacaccaccagctacaacagctgctggaccattgaaagcggaaaatacaaatacttctaaatctgcggattctctagatttggcgacaacaacttctcctcaaaattattctgaaacagcgggaaataataatactcatcatcaagatactggagaagaatctgcgtctagtggaaaattgggactaattacaaatacaattgcgggtgtagcgggattgattactggtggaagaagaactagaagagaagtaatagttaatgcgcaacctaaatgtaatccaaatctacattattggacaactcaagatgaaggtgctgcgattggactagcttggattccatattttggacctgcggcggaaggaatatatactgaaggactaatgcataatcaagatggactaatttgtggactaagacaactagcgaatgaaactacacaagcgctacaactattcttgagagcgacaacagaactaagaacttttagtattctaaatagaaaagcgattgatttcttgctacaaagatggggaggaacatgtcatattctaggaccagattgttgtattgaaccacatgattggacaaagaatattacagacaaaattgatcaaattattcatgattttgttgataaaacactaccagatcaaggagataatgataattggtggacaggatggagacaatggattccagcgggaattggagtaacaggtgtaattattgcggttattgcgctattttgtatatgtaaatttgttttttaataatttttatgtcgacctgcagtcaaactctaatgaccacatctttttttagagatgaaaaattttccacatctccttttgtagacacgactaaacattttgcagaaaaaagtttattagtgtttagataatcgtatacttcatcagtgtagatagtaaatgtgaacagataaaaggtattcttgctcaatagattggtaaattccatagaatatattaatcctttcttcttgagatcccacatcatttcaaccagagacgttttatccaatgatttacctcgtactataccacatacaaaactagattttgcagtgacgtcgtatctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaacaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 12 - DNA, 5876 bp, Ebola virus
  source 1..5876: mol_type = other DNA
gaattcggagtatacgaaccgggaaagagaagatggttaaaaataaagcgagactatttgaacgagggttccatggcagattctgccgatttagtagtactaggtgcttactatggtaaaggagcaaagggtggtatcatggcagtctttctaatgggttgttacgacgatgaatccggtaaatggaagacggttaccaagtgttcaggacacgatgataatacgttaagggagttgcaagaccaattaaagatgattaaaattaacaaggatcccaaaaaaattccagagtggttagtagttaataaaatctatattcccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatttttatggcgcgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagctccccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatttttatctcgaggccgctggtacccaacctaaaaattgaaaataaatacaaaggttcttgagggttgtgttaaattgaaagcgagaaataatcataaataagcccgggatgaaaagagtaacagtaccaacagcgccaccagcgtatgcggatataggatatccaatgtctatgctacctattaaatcttctagagcggtatctggaattcaacaaaagcaagaagtactacctggaatggatacaccatctaattctatgagaccagtagcggatgataatattgatcatacttctcatactccaaatggtgtagcgtctgcttttattctagaagcgacagtaaatgtaatttctggaccaaaagtactaatgaaacaaattccaatttggctaccactaggaattgcggatcaaaagacatattcttttgattctacaacagcggcgattatgctagcgtcttatacaattacacattttggaaaagcgaataatccactagttagagtaaatagactaggacaaggaataccagatcatccactaagactactaagaatgggaaatcaagcttttctacaagaatttgttctaccaccagtacaactaccacaatactttacatttgatctaacagcgctaaaactagtaacacaaccactaccagcggcgacatggacagatgaaactccatctaatctaagtggtgctctaagaccaggactatcttttcatccaaaactaagacctgtactactaccaggaaagactggaaagaaaggacatgtatctgatttgacagcgccagacaaaattcaaacaatagtaaatctaatgcaagacttcaaaattgtaccaattgatccagcgaaatctattattggaattgaagtaccagaactactagttcataaattgactggaaagaaaatgtctcaaaagaatggacaacctattattccagtactattgcctaaatatattggtctagatcctatttctcctggagatctaacaatggttattacaccagattatgatgattgtcattctccagcgtcttgttcttatctatctgaaaagtaataagtcgacctgcagctaatgtattagttaaatattaaaacttaccacgtaaaacttaaaatttaaaatgatatttcattgacagatagatcacacattatgaactttcaaggacttgtgttaactgacaattgcaaaaatcaatgggtcgttggaccattaataggaaaaggtggatttggtagtatttatactactaatgacaataattatgtagtaaaaatagagcccaaagctaacggatcattatttaccgaacaggcattttatactagagtacttaaaccatccgttatcgaagaatggaaaaaatctcacaatataaagcacgtaggtcttatcacgtgcaaggcatttggtctatacaaatccattaatgtggaatatcgattcttggtaattaatagattaggtgcagatctagatgcggtgatcagagccaataataatagattaccaaaaaggtcggtgatgttgatcggaatcgaaatcttaaataccatacaatttatgcacgagcaaggatattctcacggagatattaaagcgagtaatatagtcttggatcaaatagataagaataaattatatctagtggattacggattggtttctaaattcatgtcaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 13 - DNA, 7067 bp, Ebola virus
  source 1..7067: mol_type = other DNA
gaattccctgggacatacgtatatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataattttgagaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaggcgcgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagctcgaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaactcgaggccgctggtacccaacctaaaaattgaaaataaatacaaaggttcttgagggttgtgttaaattgaaagcgagaaataatcataaataagcccgggatgggaggactatctctactacaactaccaagagataagtttagaaaatcttctttctttgtttgggttataattctatttcaaaaggcgttctctatgccattgggagtagtaacaaattctacactagaagtaacagaaattgatcaactagtatgtaaagatcatctagcgtctacagatcaattgaaatctgttggattgaatctagaaggatctggtgtatctacagatattccatctgcgacaaagagatggggttttagaagtggtgtaccaccaaaagtagtatcttatgaagcgggagaatgggcggaaaattgttataatctagaaattaagaaaccagatggatctgaatgtttgccaccaccaccagatggtgttagaggatttccaagatgtagatatgttcataaagcgcaaggaacaggaccatgtcctggagattatgcgtttcataaagatggtgcattctttctatatgatagattggcgtctactgtaatatatagaggtgtaaattttgcggaaggtgtaattgcttttctaattctagcgaaacctaaagaaacatttctacaatctccaccaattagagaagcggttaattatacagaaaatacttcatcttattatgcgacatcttatctagaatatgaaattgaaaattttggagcgcaacattctacaactttgttcaaaattgataataatacttttgttagactagatagaccacatacaccacaatttttgtttcaattgaatgatacaattcatctacatcaacaactatctaatacaactggaagattgatttggacactagatgcgaatattaatgcggatattggagaatgggctttctgggaaaataagaagaatctatctgaacaactaagaggagaagaattgtcttttgaagcgctatctctaaatgaaactgaagatgatgatgcggcgtctagtagaattacaaaaggaagaatttctgatagagcgacaagacaatattctgatctagtaccaaagaatccacctggaatggttccattgcatattccagaaggagaaacaacactaccatctcaaaattctactgaaggaagaagagtatctgtaaatactcaagaaacaattacagaaacagcggcgacaattattggaacaaatggaaatcatatgcaaatttctactattggaattagaccatcttcttctcaaattccatcttctagtccaacaacagcgccatctccagaagcgcaaacaccaacaacacatacaagtggaccatctgtaatggcgacagaagaacctacaacaccaccaggatcttctccaggtccaactacagaagcgccaactctaactacaccagaaaatattacaacagctgtaaagacagtactaccacaagaatctacttctaatggactaattacatctacagtaactggaattctaggatctctaggactaagaaagagatctagaagacaaacaaatacaaaagcgactggaaaatgtaatccaaatctacattattggacagcgcaagaacaacataatgcggcgggaattgcttggattccatattttggaccaggtgctgaaggaatatatactgaaggtctaatgcataatcaaaatgcgctagtatgtggactaagacaactagcgaatgaaacaactcaagcgctacaactatttctaagagcgactacagaactaagaacatatacaattctaaatagaaaagctattgatttcttgttgagaagatggggaggaacatgtagaatattgggaccagattgttgtattgaaccacatgattggacaaagaatattactgacaaaattaatcaaattattcatgactttattgataatccactaccaaatcaagataatgatgataattggtggacaggatggagacaatggattccagcgggaataggaattactggaattattattgcgattatagcgctactatgtgtatgtaaactactatgttaataagtcgacctgcagtcaaactctaatgaccacatctttttttagagatgaaaaattttccacatctccttttgtagacacgactaaacattttgcagaaaaaagtttattagtgtttagataatcgtatacttcatcagtgtagatagtaaatgtgaacagataaaaggtattcttgctcaatagattggtaaattccatagaatatattaatcctttcttcttgagatcccacatcatttcaaccagagacgttttatccaatgatttacctcgtactataccacatacaaaactagattttgcagtgacgtcgtatctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaacaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 14 - DNA, 5807 bp, Marburg virus
  source 1..5807: mol_type = other DNA
gaattcggagtatacgaaccgggaaagagaagatggttaaaaataaagcgagactatttgaacgagggttccatggcagattctgccgatttagtagtactaggtgcttactatggtaaaggagcaaagggtggtatcatggcagtctttctaatgggttgttacgacgatgaatccggtaaatggaagacggttaccaagtgttcaggacacgatgataatacgttaagggagttgcaagaccaattaaagatgattaaaattaacaaggatcccaaaaaaattccagagtggttagtagttaataaaatctatattcccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatttttatggcgcgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagctccccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatttttatctcgaggccgctggtacccaacctaaaaattgaaaataaatacaaaggttcttgagggttgtgttaaattgaaagcgagaaataatcataaataagcccgggatggcgtctagttctaattataatacttatatgcaatatctaaatccaccaccatatgcggatcatggtgctaatcaactaattccagcggatcaactatctaatcaacatggaattacaccaaattatgttggagatctaaatctagatgatcagtttaaaggaaatgtttgtcatgcgtttacactagaagcgattattgatatttctgcgtataatgaaagaacagtaaaaggtgtaccagcttggctaccactaggaattatgtctaattttgaatatccactagcgcatacagtagcggcgctattgacaggatcttatacaattacacagtttacacataatggacaaaagtttgttagagtaaatagactaggaactggaataccagcgcatccactaagaatgctaagagaaggaaatcaagcttttattcaaaatatggttattccaagaaatttctctacaaatcagtttacttataatctaactaatctagtactatctgtacaaaagctaccagatgatgcttggagaccatctaaagataaactaattggaaatacaatgcatccagcgatttctattcatccaaatctaccaccaatagtactaccaactgtaaagaaacaagcgtatagacaacataagaatccaaataatggaccactattggcgatttctggaattctacatcaactaagagtagaaaaggtaccagaaaagacatctttgtttagaatttctctaccagcggatatgttttctgtaaaagaaggaatgatgaagaaaagaggagaatcttctccagtagtatattttcaagcgccagaaaattttccattgaatggttttaataatagacaagtagtactagcgtatgcgaatccaacactatctgcgatataataagtcgacctgcagctaatgtattagttaaatattaaaacttaccacgtaaaacttaaaatttaaaatgatatttcattgacagatagatcacacattatgaactttcaaggacttgtgttaactgacaattgcaaaaatcaatgggtcgttggaccattaataggaaaaggtggatttggtagtatttatactactaatgacaataattatgtagtaaaaatagagcccaaagctaacggatcattatttaccgaacaggcattttatactagagtacttaaaccatccgttatcgaagaatggaaaaaatctcacaatataaagcacgtaggtcttatcacgtgcaaggcatttggtctatacaaatccattaatgtggaatatcgattcttggtaattaatagattaggtgcagatctagatgcggtgatcagagccaataataatagattaccaaaaaggtcggtgatgttgatcggaatcgaaatcttaaataccatacaatttatgcacgagcaaggatattctcacggagatattaaagcgagtaatatagtcttggatcaaatagataagaataaattatatctagtggattacggattggtttctaaattcatgtcaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 15 - DNA, 7082 bp, Marburg virus
  source 1..7082: mol_type = other DNA
gaattccctgggacatacgtatatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataattttgagaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaggcgcgcctttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatgcacgagctgtacaagtaagagctcgaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaactcgaggccgctggtacccaacctaaaaattgaaaataaatacaaaggttcttgagggttgtgttaaattgaaagcgagaaataatcataaataagcccgggatgtggacaacatgtttcttcatttctctaattctaattcaaggaattaaaacactaccaattctagaaattgcgtctaatgatcaaccacaaaatgtagattctgtatgttctggaacactacaaaagactgaagatgtacatttgatgggttttacactatctggacaaaaggtagcggattctccactagaagcgtctaaaagatgggcgtttagaacaggtgtaccaccaaagaatgttgaatatacagaaggagaagaagcgaaaacttgttataatatttctgtaacagatccatctggaaaatctctactactagatccaccaactaatgttagagattatccaaaatgtaaaacaattcatcatattcaaggacaaaatccacatgcgcaaggaattgcgctacatctatggggagcattctttctatatgatagaatagcgtctacaacaatgtatagaggaaaagttttcactgaaggaaatattgcggctatgatagtaaataagacagttcacaaaatgatattttctagacaaggacaaggatatagacatatgaatctaacatctacaaataaatattggacatcttctaatggaacacaaacaaatgatacaggatgttttggaacattgcaagaatataatagtacaaagaatcaaacatgtgcgccatctaaaactccaccaccacctccaacagcgcatccagaaattaaacctacatctacaccaacagatgcgacaagattgaatacaacaaatccaaattctgatgatgaagatctaacaacatctggatctggaagtggagaacaagaaccatatacaacaagtgatgcggttacaaagcaaggactatcttctacaatgccaccaacactatctccacaacctggaactccacaacaaggtggaaataatacaaatcattctcaagatgcggcgacagaactagataatactaatacaactgcgcaaccaccaatgccatctcataatactacaactatttctactaataatacttctaaacataatctatctacattgtctgaaccacctcaaaatactactaatcctaatactcaatctatggcgactgaaaatgaaaagacttctgcgcctccaaagacaactctaccaccaactgaatctccaacaacagaaaagagtacaaataatacaaaatctccaactacaatggaacctaatacaactaatggacactttacatctccatcttctactcctaattctacaacacaacatttgatatactttagaagaaagagatctattttgtggagagaaggagatatgtttccatttctagatggattgattaatgcgccaattgattttgatccagtaccaaatacaaagacaattttcgatgaatcttcttcttctggtgcttctgcggaagaagatcaacatgcgtctagtaatattagtctaacattgtcttatctacctcatacttctgaaaatactgcgtatagtggagaaaatgagaatgattgtgatgcggaactaagaatttggagtgtacaagaagatgatctagcggcgggattgtcttggattcctttcttcggacctggaattgaaggactatatacagcgggattgattaagaatcagaataatctagtatgtagactaagaagattggcgaatcaaacagcgaaatctctagaactactactaagagtaacaactgaagaaagaacattctctttgattaatagacatgcgattgattttctattgacaagatggggaggaacatgtaaagtactaggaccagattgttgtattggaatagaagatctatctagaaatatttcagaacaaattgatcaaattaagaaagatgaacaaaaggaaggaactggatggggactaggtggaaaatggtggacatctgattggggagtactaacaaatctaggaattctactattgctatctattgcggtactaattgcgttgtcttgtatatgtagaattttcacaaagtatattggataataagtcgacctgcagtcaaactctaatgaccacatctttttttagagatgaaaaattttccacatctccttttgtagacacgactaaacattttgcagaaaaaagtttattagtgtttagataatcgtatacttcatcagtgtagatagtaaatgtgaacagataaaaggtattcttgctcaatagattggtaaattccatagaatatattaatcctttcttcttgagatcccacatcatttcaaccagagacgttttatccaatgatttacctcgtactataccacatacaaaactagattttgcagtgacgtcgtatctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaacaagcttgtctccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttcgagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggatttaggtgacactata

SEQ ID NO: 16 - DNA, 1479 bp, Lassa virus
  source 1..1479: mol_type = other DNA
atgggacaaatagtaacattcttccaagaagtaccacatgtaattgaagaagtaatgaatattgtactaattgcgctatctgtactagcggtattgaaaggattgtataatttcgcgacatgtggactagtaggactagttacatttctactactatgtggaagatcttgtacaacttctttgtataaaggagtatatgaactacaaacactagaattgaatatggaaactctaaatatgacaatgcctctatcatgtacaaagaataattctcatcattatattatggttggaaatgaaacaggactagaactaacactaacaaatacttctattattaatcataaattctgtaatctatctgatgcgcataagaagaatctatatgatcatgcgctaatgtctattatttctacatttcatctatctattccaaactttaatcaatatgaagctatgtcttgtgactttaatggtggaaagatttctgtacaatataatctaagtcattcttatgcgggagatgcggcgaatcattgtggaacagtagcgaatggtgtactacaaactttcatgagaatggcgtggggaggatcttatattgcgctagattctggaagaggaaattgggattgtattatgacatcttatcaatatctaattattcagaatacaacatgggaagatcattgtcaattctctagaccatctccaataggatatctaggactactatctcaaagaacaagagatatatatattagtagaagattgctaggaactttcacatggacactatctgattctgaaggaaaggatacacctggaggatattgtctaacaagatggatgctaattgaagcggaattgaaatgttttggaaatactgcggtagcgaaatgtaatgaaaagcatgatgaagaattttgtgatatgctaagactatttgactttaataaacaagcgattcaaagattgaaagcggaagcgcaaatgagtattcaattgataaataaagcggttaatgctttgattaatgatcaactaattatgaagaatcatctaagagatattatgggaattccatattgtaattatagtaaatattggtatctaaatcatacaacaactggaagaacatctctaccaaaatgttggctagtatctaatggatcttatctaaatgaaacacatttctctgatgatattgaacaacaagcggataatatgattacagaaatgctacaaaaggaatatatggaaagacaaggaaagacaccactaggattggtagatctatttgttttctctacatctttctatctaattagtatatttctacatctagtaaagattccaacacatagacatatagtaggaaaatcttgtccaaaaccacatagattgaatcatatgggaatatgttcttgtggattgtataaacaaccaggtgtaccagttaaatggaaaagataataa

SEQ ID NO: 17 - DNA, 303 bp, Lassa virus
  source 1..303: mol_type = other DNA
atgggaaataaacaagcgaaagcgccagaatctaaagattctccaagagcgagtctaattccagatgcgacacatctaggaccacaattttgtaaatcttgttggtttgaaaataaaggactagtagaatgtaataatcattatctatgtctaaattgtctaacactactactatctgtatctaatagatgtccaatatgcaaaatgccactaccaacaaaactaagaccatctgctgctccaacagcgccaccaacaggtgctgctgattctattagaccaccaccatattctccataataa

(19) United States
(12) Patent Application Publication
(10) Pub. No.: US 2024/0156940 A1
(43) Pub. Date: May 16, 2024

(54) REPLICATION-DEFICIENT MODIFIED VACCINIA ANKARA (MVA) EXPRESSING MARBURG VIRUS GLYCOPROTEIN (GP) AND MATRIX PROTEIN (VP40)

(71) Applicant: GeoVax, Inc., Smyrna, GA (US)

(72) Inventors: Harriet Robinson, Palo Alto, CA (US); Arban Domi, Atlanta, GA (US); Michael Hellerstein, Atlanta, GA (US)

(73) Assignee: GeoVax, Inc., Smyrna, GA (US)

(21) Appl. No.: 18/394,555

(22) Filed: Dec. 22, 2023

Related U.S. Application Data
(63) Continuation of application No. 17/584,231, filed on Jan. 25, 2022, now Pat. No. 11,896,657.
Application No. 17/584,231 is a continuation of application No. 15/543,139, filed on May 17, 2018, now Pat. No. 11,701,418.
(60) Provisional application No. 62/102,425, filed on Jan. 12, 2015.
(60) Provisional application No. 62/213,819, filed on Sep. 3, 2015.
(60) Provisional application No. 62/215,536, filed on Sep. 8, 2015.

(51) Int. Cl. A61K 39/12; C12N 15/86; C12N 15/863

(52) U.S. Cl. CPC A61K 39/12; C12N 15/86; C12N 15/863; A61K 2039/5256; C12N 2710/24141; C12N 2710/24143; C12N 2760/10034; C12N 2760/14134; C12N 2760/14234

(57) ABSTRACT

The compositions and methods are described for generating an immune response to a hemorrhagic fever virus such as ebolavirus, Marburgvirus, or arenavirus. The compositions and methods described herein relate to a modified vaccinia Ankara (MVA) vector encoding one or more viral antigens for generating a protective immune response to a member of genus Ebolavirus (such as a member of species Zaire ebolavirus), a member of genus Marburgvirus (such as a member of species Marburg marburgvirus), or a member of genus Arenavirus (such as a member of species Lassa virus) in the subject to which the vector is administered. The compositions and methods of the present invention are useful both prophylactically and therapeutically and may be used to prevent and/or treat an infection caused by ebolavirus, Marburgvirus, or arenavirus.

### CROSS REFERENCE TO RELATED APPLICATIONS

This application is a continuation of U.S. patent application Ser. No. 17/584,231, filed Jan. 25, 2022, which is a continuation of U.S. patent application Ser. No. 15/543,139, filed May 17, 2018, which is a national stage application under 35 U.S.C. § 371 of International Application No. PCT/US2016/013021, filed Jan. 12, 2016, which claims the benefit of and priority to U.S. provisional patent application U.S. 62/102,425 filed Jan. 12, 2015, U.S. provisional patent application U.S. 62/213,819 filed Sep. 3, 2015, and U.S. provisional patent application U.S. 62/215,536 filed Sep. 8, 2015. The entirety of each of these applications is hereby incorporated by reference herein for all purposes.

### FIELD OF THE INVENTION

The present invention is directed to compositions, including vaccine compositions, for generating an immune response to a hemorrhagic fever virus, as well as methods of manufacture and methods of use thereof. Hemorrhagic fever viruses include filoviruses (members of family Filoviridae), such as members of genera Ebolavirus and Marburgvirus; and arenaviruses (members of family Arenaviridae) such as members of genus Arenavirus. More specifically, the compositions and methods described herein relate to a modified vaccinia Ankara (MVA) vector encoding one or more viral antigens for generating a protective immune response in the subject to which the vector is inhibited to a member of genus Ebolavirus (such as a member of species Zaire ebolavirus), a member of genus Marburgvirus (such as a member of species Marburg marburgvirus), or a member of genus Arenavirus (such as a member of species Lassa virus). The compositions and methods of the present invention are useful both prophylactically and therapeutically.

### INCORPORATION BY REFERENCE

The contents of the xml file named “19101-015US3_ST26_2023-10-19” which was created on Oct. 19, 2023, and is 68.8 KB in size, are hereby incorporated by reference in their entirety.

### BACKGROUND OF THE INVENTION

The Filoviridae family is composed of three genera, Ebolavirus, Marburgvirus, and Cuevavirus. Genera Ebolavirus and Marburgvirus include highly pathogenic and virulent viruses causing rapidly fatal hemorrhagic fever in humans and non-human primates. Genus Marburgvirus has only one known species (Marburg marburgvirus), whereas genus Ebolavirus is more variable and has five known species.

The five distinct species of genus Ebolavirus include Zaire ebolavirus, Sudan ebolavirus, Taï Forest ebolavirus, Bundibugyo ebolavirus, and Reston ebolavirus. (Carroll et al., J. Virol., 87(5):2608-2616 (2013). Four of these species (Zaire ebolavirus, Sudan ebolavirus, Taï Forest ebolavirus, and Bundibugyo ebolavirus), cause fatal disease in humans.

Known viruses belonging to species Zaire ebolavirus are commonly referred to as Ebola viruses. Ebola virus is abbreviated as EBOV. Known viruses belonging to species Sudan ebolavirus are commonly referred to as Sudan viruses. Sudan virus is abbreviated as SUDV. Known viruses belonging to species Taï Forest ebolavirus are commonly referred to as Tai Forest viruses. Tai Forest virus is abbreviated as TAFV. Known viruses belonging to species Bundibugyo ebolavirus are commonly referred to as Bundibugyo viruses. Bundibugyo virus is abbreviated as BDBV. Known viruses belonging to species Marburg marburgvirus include Marburg virus (MARV) and Ravn virus (RAVV). (Kuhn et al., Viruses, 6:3663-3682 [2014]) Various forms of filovirus nomenclature and abbreviation have been used in the past. Other known abbreviations for members of this group include ZEBOV for Ebola virus, SEBOV for Sudan virus, CIEBOV for Taï Forest virus, BEBOV for Bundibugyo virus, and REBOV for Reston virus.

In this application, the terms “ebolavirus” or “Ebolavirus” (single word, not italicized) will be used to refer to any member of genus Ebolavirus, while the terms “marburgvirus” or “Marburgvirus” will be used to refer to any member of genus Marburgvirus.

The genetic organization of filoviruses is similar, each containing seven genes in a linear, single-stranded, negative-sense RNA genome. Among the viral proteins expressed from the ebolavirus genome, the envelope glycoprotein exists in three alternative forms: a 50-70 kilodalton (kDa) secreted protein encoded by the viral genome (sGP), a 130 kDa transmembrane glycoprotein (GP), and a small secreted glycoprotein (ssGP), which is a smaller (approximately 50 kDa) version of the secreted glycoprotein. Transcripts for the full-length glycoprotein and ssGP are generated by RNA editing. The functions of sGP and ssGP are unknown, while the transmembrane protein mediates viral entry. (Mehedi, M. et al., J. Virol. 85:5406-5414 (2011); Peters, C. J. et al., Filoviridae: Marburg and Ebola Viruses. in Fields Virology. (eds., Fields, B. N., Knipe, D. M. & Howley, P. M.) 1161-1176 (Philadelphia, Lippincott-Raven, 1996); Sanchez, A. et al., PNAS (USA) 93:3602-3607 (1996). Other gene products include the nucleoprotein (NP), matrix proteins VP24 and VP40, the transcription factor VP30, the polymerase cofactor VP35, and the viral polymerase L (Biedenkopf, N. et al., J. Biol. Chem. 288:11165-11174 (2013); Nanbo, A. et al., Scientific Reports 3, doi: 10.1038/srep01206 (2013); reviewed in Peters, C. J. et al., Filovirdae: Marburg and Ebola Viruses. in Fields Virology. (eds., Fields, B. N., Knipe, D. M. & Howley, P. M.) 1161-1176 (Philadelphia, Lippincott-Raven, 1996)). Proteins expressed by marburgviruses are very similar, but marburgvirus does not express sGP or ssGP (Radoshitzsky, S. R. et al.; J. Virol. 85:8502-8513 (2011)).

Although spontaneous variation of their RNA sequence does occur in nature, there appears to be less nucleotide polymorphism within ebolavirus subtypes than among other RNA viruses (Sanchez, A. et al., PNAS (USA) 93:3602-3607 (1996)).

Since Ebola virus was discovered in 1976, more than 20 outbreaks have occurred (source: cdc.gov). The development of countermeasures against filoviruses have largely focused on SUDV and EBOV, the two species that have historically been responsible for nearly all ebolavirus outbreaks. To date, however, no approved vaccine or therapeutic product is available for Filovirus infections. As such, medical professionals have no means to prevent infection other than the traditional methods of isolation and sanitation, and no means to treat infected patients.

Arenaviridae comprises a family of viruses whose members are generally associated with rodent-transmitted diseases in humans. Arenaviruses are divided into two groups: the New World or Tacaribe complex and the Old World or LCM/Lassa complex. Arenavirus infections are relatively common in humans in some areas of the world and can cause severe illnesses.

Lassa virus (LASV) is an arenavirus that causes Lassa hemorrhagic fever, a type of viral hemorrhagic fever (VHF), in human and non-human primates. Lassa virus is an emerging virus and a select agent, requiring containment under Biosafety Level 4 or an equivalent standard. LASV is endemic in West African countries, especially Sierra Leone, the Republic of Guinea, Nigeria, and Liberia, where the annual incidence of infection is between 300,000 and 500,000 cases, resulting in 5,000 deaths per year (Kyei et al. (2015), BMC Infectious Diseases 15:217).

Lassa viruses are enveloped, single-stranded, bisegmented, ambisense RNA viruses (Lashley, Felissa R., and Jerry D. Durham. Emerging Infectious Diseases: Trends and Issues. New York: Springer Pub., 2002). Their genome is contained in two RNA segments that code for two proteins each, one in each sense, for a total of four viral proteins (Ridley, Matt. Genome: The Autobiography of a Species in 23 Chapters. New York: HarperCollins, 1999). The large segment encodes a small zinc-binding protein (Z) that regulates transcription and replication, and the RNA polymerase (L). The small segment encodes the nucleoprotein (NP) and the surface glycoprotein precursor (GP, also known as the viral spike), which is proteolytically cleaved into the envelope glycoproteins GP1 and GP2 that bind to the alpha-dystroglycan receptor and mediate host cell entry (Cornu, T. I.; De La Torre, J. C. (2001). RING Finger Z Protein of Lymphocytic Choriomeningitis Virus (LCMV) Inhibits Transcription and RNA Replication of an LCMV S-Segment Minigenome“. Journal of Virology 75 (19): 9415-9426; Djavani M, et al. (September 1997). “Completion of the Lassa fever virus sequence and identification of a RING finger open reading frame at the L RNA 5′ End.”. Virology 235 (2): 414-8; Cao, W.; Henry, M. D.; Borrow, P.; Yamada, H.; Elder, J. H.; Ravkov, E. V.; Nichol, S. T.; Compans, R. W.; Campbell, K. P.; Oldstone, M. B. (1998). “Identification of -Dystroglycan as a Receptor for Lymphocytic Choriomeningitis Virus and Lassa Fever Virus”. Science 282 (5396): 2079-2081)

The pathogenesis of the Lassa virus remains unclear, but it has been shown that the main targets of the virus are antigen-presenting cells (mainly dendritic cells) and endothelial cells (Mahanty, S.; Hutchinson, K.; Agarwal, S.; McRae, M.; Rollin, P. E.; Pulendran, B. (2003). “Cutting edge: Impairment of dendritic cells and adaptive immunity by Ebola and Lassa viruses”. Journal of immunology, 170 (6): 2797-2801; Baize, S.; Kaplon, J.; Faure, C.; Pannetier, D.; Georges-Courbot, M. C.; Deubel, V. (2004). “Lassa virus infection of human dendritic cells and macrophages is productive but fails to activate cells”. Journal of immunology (Baltimore, Md.: 1950) 172 (5): 2861-2869). Also, it is reported that Lassa virus prevents a host's innate immune system by NP activity. NP encoded in Lassa virus is essential in viral replication and transcription, but it also suppresses host innate interferon (IFN) response by inhibiting translocation of IRF-3. NP of Lassa virus is reported to have an exonuclease activity to only dsRNAs. dsRNA exonuclease activity of the NP leads to counteract IFN responses by digesting the PAMP which leads to the evasion of host immune responses.

Currently there is no US licensed vaccine for humans against the Lassa virus. Lassa fever is one of the most prevalent viral hemorrhagic fevers in West Africa responsible for thousands of deaths annually.

What is therefore needed are vaccine compositions and methods of use to prevent and treat disease caused by hemorrhagic fever virus infection, such as an ebolavirus, marburgvirus, or Lassa virus infection.

### SUMMARY OF THE INVENTION

The compositions and methods of the invention described herein are useful for generating an immune response to at least one hemorrhagic fever virus in a subject in need thereof. Advantageously, the compositions and methods may be used prophylactically to immunize a subject against ebolavirus, marburgvirus or Lassa virus infection, or used therapeutically to prevent, treat or ameliorate the onset and severity of disease.

In a first aspect, the present invention is a recombinant modified vaccinia Ankara (MVA) vector comprising a glycoprotein sequence and a matrix protein sequence, wherein both the glycoprotein sequence and matrix protein sequence are inserted into the MVA vector under the control of promoters compatible with poxvirus expression systems.

In one embodiment, the glycoprotein sequence and the matrix protein sequence are inserted into one or more deletion sites of the MVA vector.

In one embodiment, the glycoprotein sequence and the matrix protein sequence are inserted into the MVA vector in a natural deletion site, a modified natural deletion site, or between essential or non-essential MVA genes.

In another embodiment, the glycoprotein sequence and the matrix protein sequence are inserted into the same natural deletion site, a modified natural deletion site, or between the same essential or non-essential MVA genes

In another embodiment, the glycoprotein sequence and the matrix protein sequence are inserted into a deletion site selected from I, II, III, IV, V or VI and the matrix protein sequence is inserted into a deletion site selected from I, II, III, IV, V or VI.

In another embodiment, the glycoprotein sequence and the matrix protein sequence are inserted into different natural deletion sites, modified deletion sites, or between different essential or non-essential MVA genes.

In another embodiment, the glycoprotein sequence is inserted in a first deletion site and matrix protein sequence is inserted into a second deletion site.

In a particular embodiment, the glycoprotein sequence is inserted between two essential and highly conserved MVA genes; and the matrix protein sequence is inserted into a restructured and modified deletion III.

In one embodiment, the deletion III is modified to remove non-essential sequences and insert the matrix protein sequence between essential genes.

In a particular embodiment, the matrix protein sequence is inserted between MVA genes, I8R and G1L.

In a particular embodiment, the glycoprotein sequence is inserted between two essential and highly conserved MVA genes to limit the formation of viable deletion mutants.

In a particular embodiment, the glycoprotein protein sequence is inserted between MVA genes, I8R and G1L.

In one embodiment, the promoter is selected from the group consisting of Pm2H5, Psyn II, and mH5 promoters or combinations thereof.

In one embodiment, the glycoprotein sequence is optimized. In a particular embodiment, the glycoprotein sequence is optimized by changing selected codons to other synonymous codons that are optimal for protein expression by MVA, interrupting homopolymer stretches using silent mutations, interrupting transcription terminator motifs using silent mutations, or leading to expression of the transmembrane (rather than secreted) form of glycoprotein, and combinations thereof.

In one embodiment, the recombinant MVA viral vector expresses glycoprotein and matrix proteins that assemble into VLPs.

In one embodiment, the glycoprotein sequence and the matrix protein sequence are from a Filovirus species selected from the group of consisting of Zaire ebolavirus, Sudan ebolavirus, Taï forest ebolavirus, Bundibugyo ebolavirus, Reston ebolavirus, and Marburg marburgvirus, or a combination thereof.

In a particular embodiment, the glycoprotein sequence and the matrix protein sequence are from a Zaire ebolavirus.

In a particular embodiment, the glycoprotein sequence and the matrix protein sequence are from a 2014 epidemic strain of Zaire ebolavirus.

In a particular embodiment, the glycoprotein sequence and the matrix protein sequence are from a Sudan ebolavirus.

In a particular embodiment, the glycoprotein sequence and the matrix protein sequence are from a Bundibugyo ebolavirus.

In a particular embodiment, the glycoprotein sequence is from Zaire ebolavirus and the matrix protein sequence is from a Sudan ebolavirus.

In a particular embodiment, the glycoprotein sequence is from Zaire ebolavirus and the matrix protein sequence is from Bundibugyo ebolavirus.

In a particular embodiment, the glycoprotein sequence is from Sudan ebolavirus and the matrix protein sequence is from a Zaire ebolavirus.

In a particular embodiment, the glycoprotein sequence is from a Sudan ebolavirus and the matrix protein sequence is from a Bundibugyo ebolavirus.

In a particular embodiment, the glycoprotein sequence is from a Bundibugyo ebolavirus and the matrix protein sequence is from a Sudan ebolavirus.

In a particular embodiment, the glycoprotein sequence is from a Bundibugyo ebolavirus and the matrix protein sequence is from a Zaire ebolavirus.

In a particular embodiment, the glycoprotein sequence and the matrix protein sequence are from a Marburg marburgvirus.

In a particular embodiment, the glycoprotein sequence and the matrix protein sequence are from a Lassa virus.

In one embodiment, the recombinant MVA viral vector expresses Lassa virus glycoprotein and Z proteins that assemble into VLPs.

In one embodiment, the recombinant MVA viral vector expresses Lassa virus glycoprotein, NP and Z proteins that assemble into VLPs.

In a second aspect, the present invention is a pharmaceutical composition comprising the recombinant MVA vector of the present invention and a pharmaceutically acceptable carrier.

In one embodiment, the recombinant MVA vector is formulated for intraperitoneal, intramuscular, intradermal, epidermal, mucosal or intravenous administration.

In a third aspect, the present invention is a pharmaceutical composition comprising a first recombinant MVA vector and a second recombinant MVA vector, each comprising a glycoprotein sequence and a matrix protein sequence, wherein (i) the glycoprotein sequence of the first recombinant MVA vector is different than the glycoprotein sequence of the second recombinant MVA vector and/or (ii) the matrix protein sequence of the first recombinant MVA vector is different than the matrix protein sequence of the second recombinant MVA vector.

In a particular embodiment, the glycoprotein sequence of the first recombinant MVA vector is from a different species than the glycoprotein sequence of the second recombinant MVA vector.

In a particular embodiment, the glycoprotein sequences of the recombinant MVA vectors are from a Zaire ebolavirus and a Bundibugyo ebolavirus.

In a particular embodiment, the glycoprotein sequences of the recombinant MVA vectors are from a Zaire ebolavirus and a Sudan ebolavirus.

In a particular embodiment, the glycoprotein sequences of the recombinant MVA vectors are from a Sudan ebolavirus and a Bundibugyo ebolavirus.

In a particular embodiment, the glycoprotein sequences of the recombinant MVA vectors are from a Zaire ebolavirus and a Marburg marburgvirus.

In a particular embodiment, the glycoprotein sequences of the recombinant MVA vectors are from a Sudan ebolavirus and a Marburg marburgvirus.

In a particular embodiment, the glycoprotein sequences of the recombinant MVA vectors are from a Bundibugyo ebolavirus and a Marburg marburgvirus.

In a particular embodiment, the glycoprotein sequences of the recombinant MVA vectors are from a Zaire ebolavirus and a Lassa virus.

In a particular embodiment, the glycoprotein sequences of the recombinant MVA vectors are from a Sudan ebolavirus and a Lassa virus.

In a particular embodiment, the glycoprotein sequences of the recombinant MVA vectors are from a Bundibugyo ebolavirus and a Lassa virus.

In a particular embodiment, the glycoprotein sequences of the recombinant MVA vectors are from a Marburg marburgvirus and a Lassa virus.

In another particular embodiment, the matrix protein sequence of the first recombinant MVA vector is from a different species than the matrix protein sequence of the second recombinant MVA vector.

In a particular embodiment, the matrix protein sequences of the recombinant MVA vectors are from a Zaire ebolavirus and a Bundibugyo ebolavirus.

In a particular embodiment, the matrix protein sequences of the recombinant MVA vectors are from a Zaire ebolavirus and a Sudan ebolavirus.

In a particular embodiment, the matrix protein sequences of the recombinant MVA vectors are from a Sudan ebolavirus and a Bundibugyo ebolavirus.

In a particular embodiment, the matrix protein sequences of the recombinant MVA vectors are from a Zaire ebolavirus and a Marburg marburgvirus.

In a particular embodiment, the matrix protein sequences of the recombinant MVA vectors are from a Sudan ebolavirus and a Marburg marburgvirus.

In a particular embodiment, the matrix protein sequences of the recombinant MVA vectors are from a Bundibugyo ebolavirus and a Marburg marburgvirus.

In a particular embodiment, the matrix protein sequences of the recombinant MVA vectors are from a Zaire ebolavirus and a Lassa virus.

In a particular embodiment, the matrix protein sequences of the recombinant MVA vectors are from a Sudan ebolavirus and a Lassa virus.

In a particular embodiment, the matrix protein sequences of the recombinant MVA vectors are from a Bundibugyo ebolavirus and a Lassa virus.

In a particular embodiment, the matrix protein sequences of the recombinant MVA vectors are from a Marburg marburgvirus and a Lassa virus.

In a fourth aspect, the present invention is a pharmaceutical composition comprising three or more recombinant MVA vectors each comprising a glycoprotein sequence and a matrix protein sequence, wherein (i) the three or more recombinant MVA vectors contain different glycoprotein sequences and/or (ii) the three recombinant MVA vectors contain different matrix protein sequences.

In a particular embodiment, the glycoprotein sequence and matrix sequence of each recombinant vector are from the same species.

In a particular embodiment, the glycoprotein sequences of the three or more recombinant MVA vectors are from different species.

In a particular embodiment, the glycoprotein sequences of the three or more recombinant MVA vectors are from a Zaire ebolavirus, a Sudan ebolavirus, and a Bundibugyo ebolavirus.

In a particular embodiment, the glycoprotein sequences of the three or more recombinant MVA vectors are from a Zaire ebolavirus, a Sudan ebolavirus, and a Marburg marburgvirus.

In a particular embodiment, the glycoprotein sequences of the three or more recombinant MVA vectors are from a Zaire ebolavirus, a Sudan ebolavirus, and a Lassa virus.

In a particular embodiment, the glycoprotein sequences of the three or more recombinant MVA vectors are from a Zaire ebolavirus, a Bundibugyo ebolavirus, and a Marburg marburgvirus.

In a particular embodiment, the glycoprotein sequences of the three or more recombinant MVA vectors are from a Zaire ebolavirus, a Bundibugyo ebolavirus, and a Lassa virus.

In a particular embodiment, the glycoprotein sequences of the three or more recombinant MVA vectors are from a Zaire ebolavirus, a Marburg marburgvirus, and a Lassa virus.

In a particular embodiment, the glycoprotein sequences of the three or more recombinant MVA vectors are from a Sudan ebolavirus, a Bundibugyo ebolavirus, and a Marburg marburgvirus.

In a particular embodiment, the glycoprotein sequences of the three or more recombinant MVA vectors are from a Sudan ebolavirus, a Bundibugyo ebolavirus, and a Lassa virus.

In a particular embodiment, the glycoprotein sequences of the three or more recombinant MVA vectors are from a Sudan ebolavirus, a Marburg marburgvirus, and a Lassa virus.

In a particular embodiment, the glycoprotein sequences of the three or more recombinant MVA vectors are from a Bundibugyo ebolavirus, a Marburg marburgvirus, and a Lassa virus.

In a particular embodiment, the glycoprotein sequences of the three or more recombinant MVA vectors are from a Zaire ebolavirus, a Sudan ebolavirus, a Bundibugyo ebolavirus, and a Marburg marburgvirus.

In a particular embodiment, the glycoprotein sequences of the three or more recombinant MVA vectors are from a Zaire ebolavirus, a Sudan ebolavirus, a Bundibugyo ebolavirus, and a Lassa virus.

In a particular embodiment, the glycoprotein sequences of the three or more recombinant MVA vectors are from a Sudan ebolavirus, a Bundibugyo ebolavirus, a Marburg marburgvirus, and a Lassa virus.

In a particular embodiment, the glycoprotein sequences of the three or more recombinant MVA vectors are from a Zaire ebolavirus, a Bundibugyo ebolavirus, a Marburg marburgvirus, and a Lassa virus.

In a particular embodiment, the glycoprotein sequences of the three or more recombinant MVA vectors are from a Zaire ebolavirus, a Sudan ebolavirus, a Bundibugyo ebolavirus, a Marburg marburgvirus, and a Lassa virus.

In a particular embodiment, the matrix protein sequences of the three or more recombinant MVA vectors are from different species.

In a particular embodiment, the matrix protein sequences of the three or more recombinant MVA vectors are from a Zaire ebolavirus, a Sudan ebolavirus, and a Bundibugyo ebolavirus.

In a particular embodiment, the matrix protein sequences of the three or more recombinant MVA vectors are from a Zaire ebolavirus, a Sudan ebolavirus, and a Marburg marburgvirus.

In a particular embodiment, the matrix protein sequences of the three or more recombinant MVA vectors are from a Zaire ebolavirus, a Sudan ebolavirus, and a Lassa virus.

In a particular embodiment, the matrix protein sequences of the three or more recombinant MVA vectors are from a Zaire ebolavirus, a Bundibugyo ebolavirus, and a Marburg marburgvirus.

In a particular embodiment, the matrix protein sequences of the three or more recombinant MVA vectors are from a Zaire ebolavirus, a Bundibugyo ebolavirus, and a Lassa virus.

In a particular embodiment, the matrix protein sequences of the three or more recombinant MVA vectors are from a Zaire ebolavirus, a Marburg marburgvirus, and a Lassa virus.

In a particular embodiment, the matrix protein sequences of the three or more recombinant MVA vectors are from a Sudan ebolavirus, a Bundibugyo ebolavirus, and a Marburg marburgvirus.

In a particular embodiment, the matrix protein sequences of the three or more recombinant MVA vectors are from a Sudan ebolavirus, a Bundibugyo ebolavirus, and a Lassa virus.

In a particular embodiment, the matrix protein sequences of the three or more recombinant MVA vectors are from a Sudan ebolavirus, a Marburg marburgvirus, and a Lassa virus.

In a particular embodiment, the matrix protein sequences of the three or more recombinant MVA vectors are from a Bundibugyo ebolavirus, a Marburg marburgvirus, and a Lassa virus.

In a particular embodiment, the matrix protein sequences of the three or more recombinant MVA vectors are from a Zaire ebolavirus, a Sudan ebolavirus, a Bundibugyo ebolavirus, and a Marburg marburgvirus.

In a particular embodiment, the matrix protein sequences of the three or more recombinant MVA vectors are from a Zaire ebolavirus, a Sudan ebolavirus, a Bundibugyo ebolavirus, and a Lassa virus.

In a particular embodiment, the matrix protein sequences of the three or more recombinant MVA vectors are from a Sudan ebolavirus, a Bundibugyo ebolavirus, a Marburg marburgvirus, and a Lassa virus.

In a particular embodiment, the matrix protein sequences of the three or more recombinant MVA vectors are from a Zaire ebolavirus, a Bundibugyo ebolavirus, a Marburg marburgvirus, and a Lassa virus.

In a particular embodiment, the matrix protein sequences of the three or more recombinant MVA vectors are from a Zaire ebolavirus, a Sudan ebolavirus, a Bundibugyo ebolavirus, a Marburg marburgvirus, and a Lassa virus.

In a fifth aspect, the present invention is a method of inducing an immune response in a subject in need thereof, said method comprising administering the composition of the present invention to the subject in an amount sufficient to induce an immune response.

In one embodiment, the immune response is a humoral immune response, a cellular immune response or a combination thereof.

In a particular embodiment, the immune response comprises production of binding antibodies against the ebolavirus, marburgvirus, or Lassa virus.

In a particular embodiment, the immune response comprises production of neutralizing antibodies against the ebolavirus, marburgvirus, or Lassa virus.

In a particular embodiment, the immune response comprises production of non-neutralizing antibodies against the ebolavirus, marburgvirus, or Lassa virus.

In a particular embodiment, the immune response comprises production of a cell-mediated immune response against the ebolavirus, marburgvirus, or Lassa virus.

In a particular embodiment, the immune response comprises production of neutralizing and non-neutralizing antibodies against the ebolavirus, marburgvirus, or Lassa virus.

In a particular embodiment, the immune response comprises production of neutralizing antibodies and cell-mediated immunity against the ebolavirus, marburgvirus, or Lassa virus.

In a particular embodiment, the immune response comprises production of non-neutralizing antibodies and cell-mediated immunity against the ebolavirus, marburgvirus, or Lassa virus.

In a particular embodiment, the immune response comprises production of neutralizing antibodies, non-neutralizing antibodies, and cell-mediated immunity against the ebolavirus, marburgvirus, or Lassa virus.

In a sixth aspect, the present invention is a method of preventing a hemorrhagic fever virus infection in a subject in need thereof, said method comprising administering the recombinant MVA vector of the present invention to the subject in a prophylactically effective amount.

In one embodiment, the hemorrhagic fever infection is an ebolavirus, marburgvirus, or Lassa virus infection.

In one embodiment, the method prevents infection by a Zaire ebolavirus.

In another embodiment, the method prevents infection by a Sudan ebolavirus.

In another embodiment, the method prevents infection by a Bundibugyo ebolavirus.

In another embodiment, the method prevents infection by a Marburg marburgvirus.

In another embodiment, the method prevents infection by a Lassa virus.

In yet another embodiment, the method prevents infection by more than one species of hemorrhagic fever virus, e.g., a Zaire ebolavirus and a Sudan ebolavirus or a Zaire ebolavirus and a Marburg marburgvirus or a Zaire ebolavirus and a Lassa virus.

In a seventh aspect, the present invention is a method of inducing an immune response in a subject in need thereof, said method comprising administering the recombinant MVA vector of the present invention to the subject in a prophylactically effective amount.

In one embodiment, the immune response is considered a surrogate marker for protection.

In one embodiment, the method induces an immune response against a Zaire ebolavirus.

In another embodiment, the method induces an immune response against a Sudan ebolavirus.

In another embodiment, the method induces an immune response to a Bundibugyo ebolavirus.

In another embodiment, the method induces an immune response to a Marburg marburgvirus.

In another embodiment, the method induces an immune response to a Lassa virus.

In yet another embodiment, the method induces an immune response to more than one species of hemorrhagic fever virus, e.g., a Zaire ebolavirus and a Sudan ebolavirus or a Zaire ebolavirus and a Marburg marburgvirus or a Sudan ebolavirus and a Lassa virus

In an eighth aspect, the present invention is a method of treating hemorrhagic fever virus infection in a subject in need thereof, said method comprising administering the recombinant MVA vector in a therapeutically effective amount to the subject.

In one embodiment, the hemorrhagic fever virus infection is caused by an ebolavirus, an marburgvirus or Lassa virus.

In one embodiment, the subject is exposed to hemorrhagic fever virus, but not yet symptomatic of hemorrhagic fever virus infection. In a particular embodiment, treatment results in prevention of a symptomatic infection.

In another embodiment, the subject was recently exposed but exhibits minimal symptoms of infections.

In another embodiment, the method results in amelioration of at least one symptom of infection.

In one embodiment, the symptom of infection is fever and/or hemorrhagic bleeding.

In another embodiment, the method results in reduction or elimination of the subject's ability to transmit the infection to an uninfected subject.

In one embodiment, the method prevents or ameliorates a Zaire ebolavirus infection.

In another embodiment, the method prevents or ameliorates a Sudan ebolavirus infection.

In one embodiment, the method prevents or ameliorates a Bundibugyo ebolavirus infection.

In another embodiment, the method prevents or ameliorates a Marburg marburgvirus infection.

In another embodiment, the method prevents or ameliorates a Lassa virus infection.

In yet another embodiment, the method prevents or ameliorates infections resulting from more than one species of hemorrhagic fever virus, e.g., Zaire ebolavirus and Sudan ebolavirus infections or Zaire ebolavirus and Marburg marburgvirus infections or Bundibugyo ebolavirus and Lassa virus infections.

In a ninth aspect, the present invention is a method manufacturing a recombinant modified vaccinia Ankara (MVA) vector comprising inserting at least one glycoprotein sequence and at least one matrix protein sequence into the MVA vector operably linked to promoters compatible with poxvirus expression systems.

In one embodiment, the matrix sequence is VP40, and the GP sequence and the VP40 sequence are from a Filovirus species selected from the group consisting of Zaire ebolavirus, Sudan ebolavirus, nil forest ebolavirus, Bundibugyo ebolavirus, Reston ebolavirus, and Marburg marburgvirus, or a combination thereof.

In a particular embodiment, the GP sequence and the VP40 sequence are from a Zaire ebolavirus.

In a particular embodiment, the GP sequence and the VP40 sequence are from a 2014 epidemic strain of Zaire ebolavirus.

In a particular embodiment, the GP sequence and the VP40 sequence are from a Sudan ebolavirus.

In a particular embodiment, the GP sequence and the VP40 sequence are from a Bundibugyo ebolavirus.

In a particular embodiment, the GP sequence is from Zaire ebolavirus and the VP40 sequence is from a Sudan ebolavirus.

In a particular embodiment, the GP sequence is from Zaire ebolavirus and the VP40 sequence is from Bundibugyo ebolavirus.

In a particular embodiment, the GP sequence is from Sudan ebolavirus and the VP40 sequence is from a Zaire ebolavirus.

In a particular embodiment, the GP sequence is from a Sudan ebolavirus and the VP40 sequence is from a Bundibugyo ebolavirus.

In a particular embodiment, the GP sequence is from a Bundibugyo ebolavirus and the VP40 sequence is from a Sudan ebolavirus.

In a particular embodiment, the GP sequence is from a Bundibugyo ebolavirus and the VP40 sequence is from a Zaire ebolavirus.

In a particular embodiment, the GP sequence and the VP40 sequence are from a Marburg marburgvirus.

In a particular embodiment, the GP sequence is from a Lassa virus, and the matrix protein sequence is a Z sequence from a Lassa virus.

In a particular embodiment, the GP sequence is from a Lassa virus, the matrix protein sequence is a Z sequence from a Lassa virus and further comprises a nucleoprotein (NP) sequence from Lassa virus.

In one embodiment, the recombinant MVA viral vector expresses Lassa virus glycoprotein and matrix proteins that assemble into VLPs.

### BRIEF DESCRIPTION OF THE DRAWINGS

FIG. 1 is a simple line drawing illustrating the design of the MVA vectors.

The numbering illustrates the positions (in kilobase pairs) of the various elements in the genome of the MVA vaccine vector. For clarity and brevity, the diagram is not to scale; pairs of diagonal lines indicate a section of the MVA genome that is not illustrated because its contents are not relevant to the invention. Arrows labeled “gp” and “vp40” illustrate the positions of the genes encoding GP and VP40, respectively for use with ebolavirus or Marburgvirus sequences. Rectangles labeled “I8R” and “GIL” indicate the positions of the two MVA genetic elements flanking the gene encoding GP. Rectangles labeled “A50R” and “B1R” indicate the positions of the two MVA genetic elements flanking the gene encoding VP40.

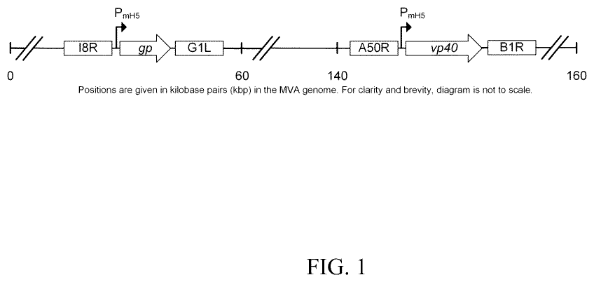

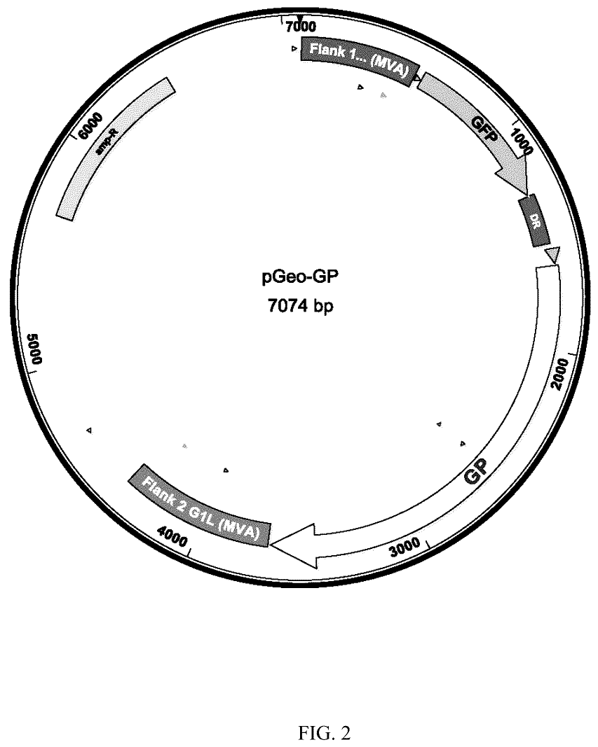

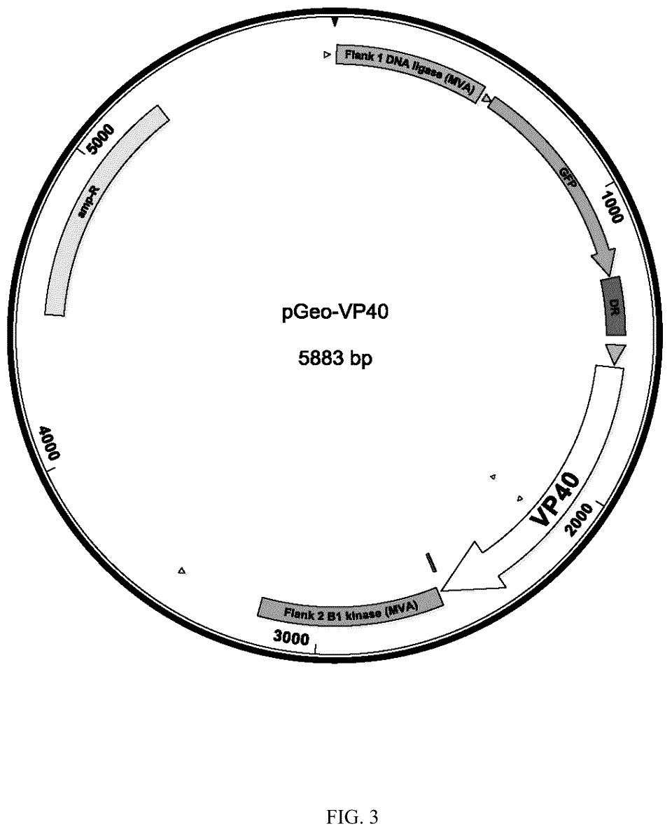

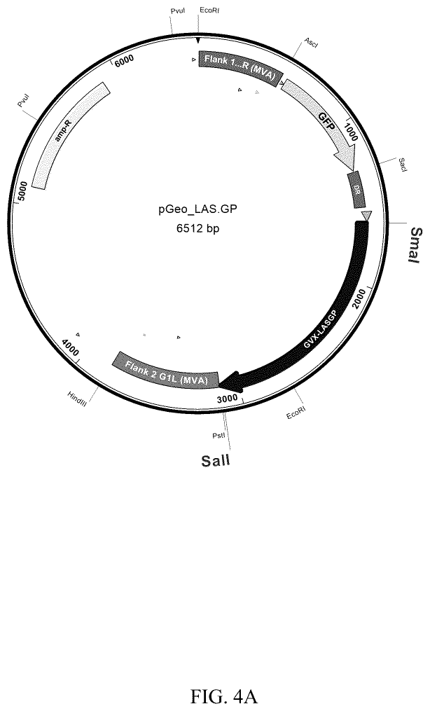

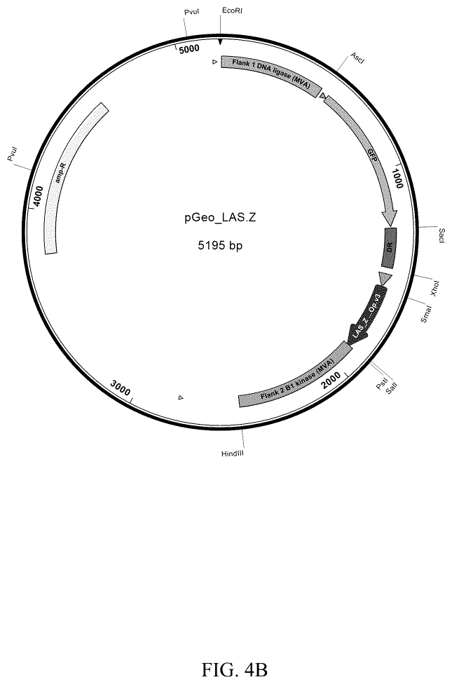

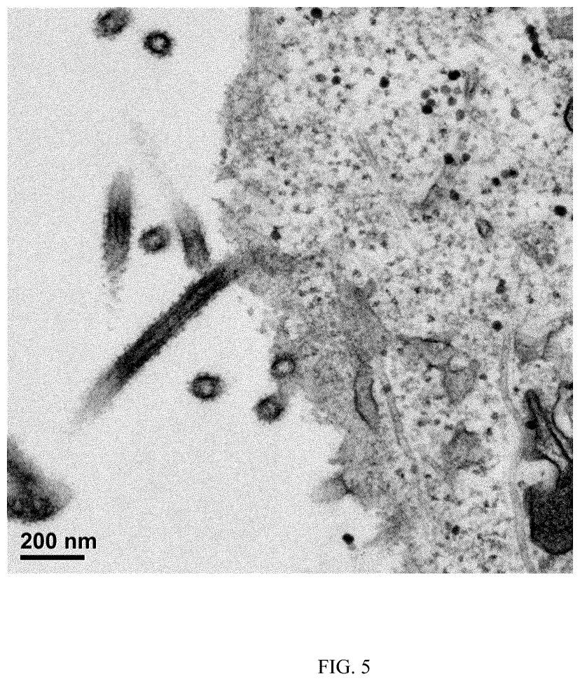

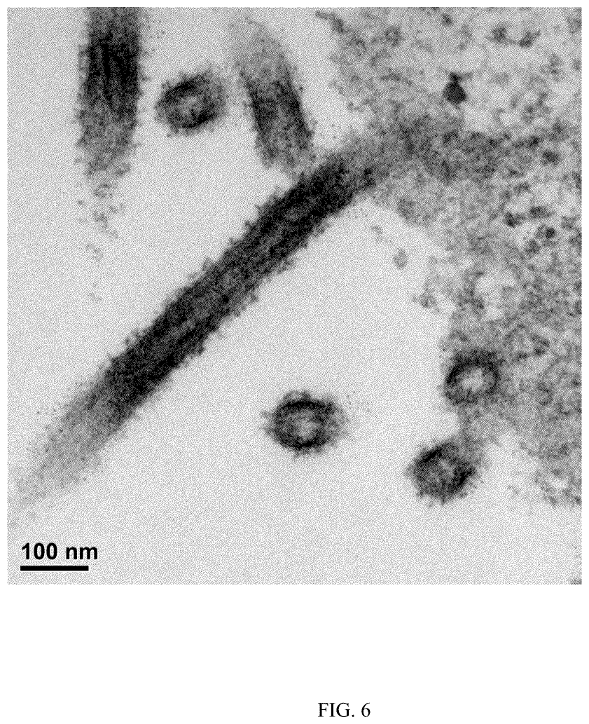

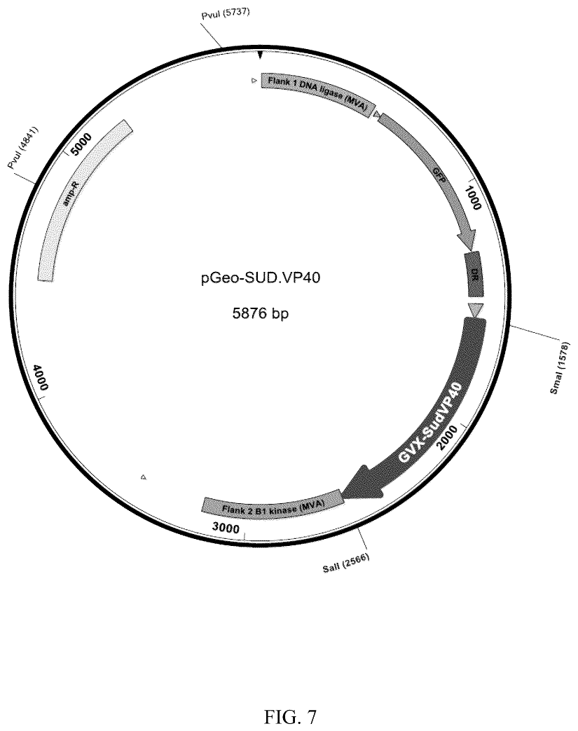

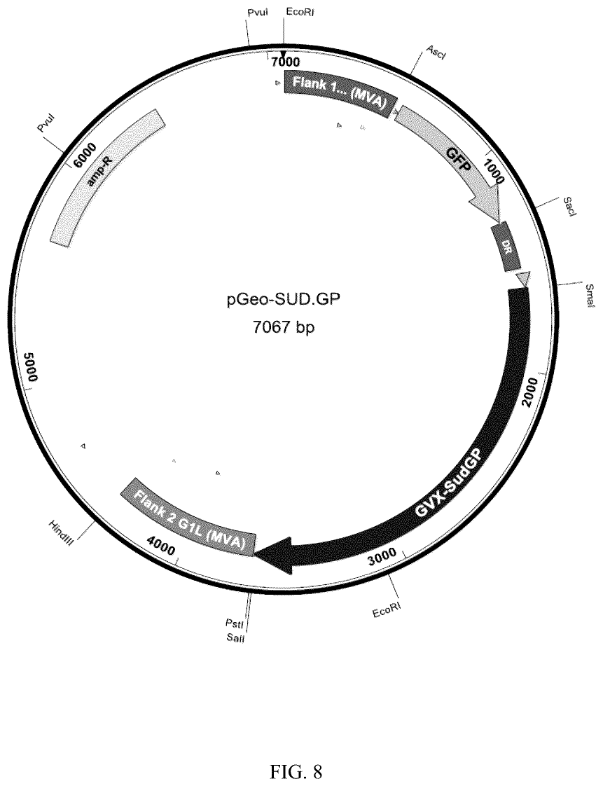

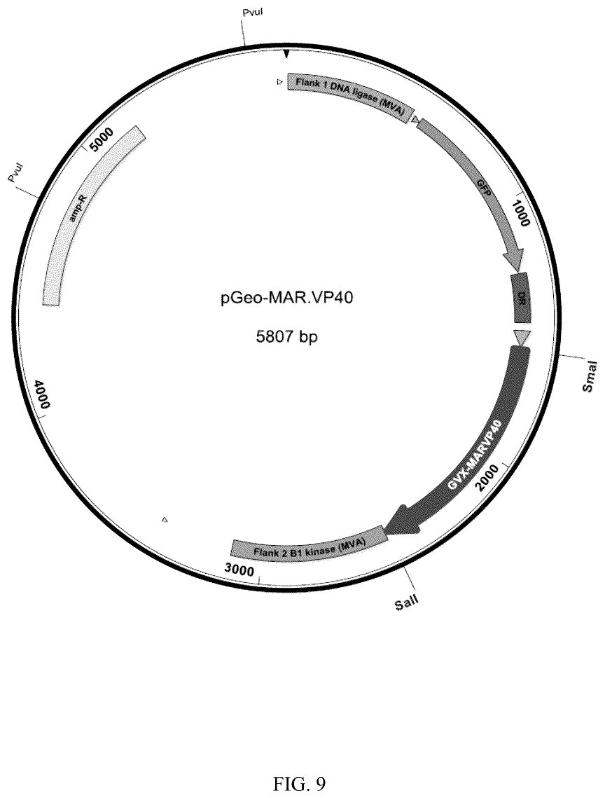

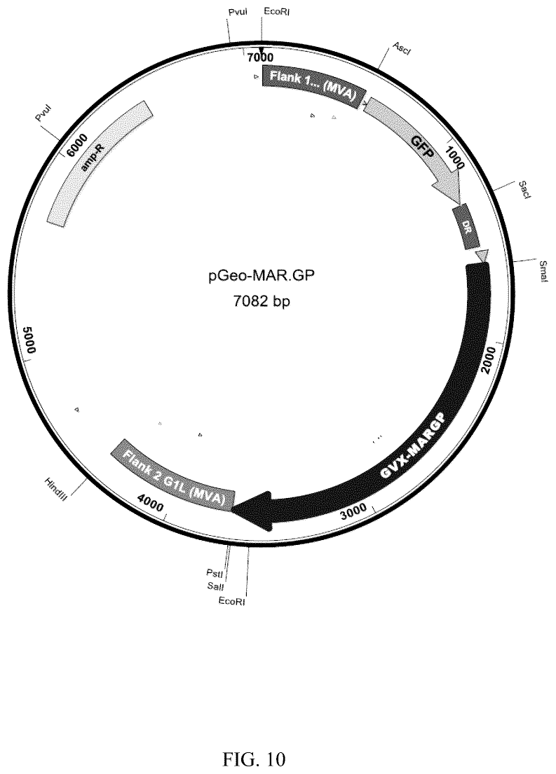

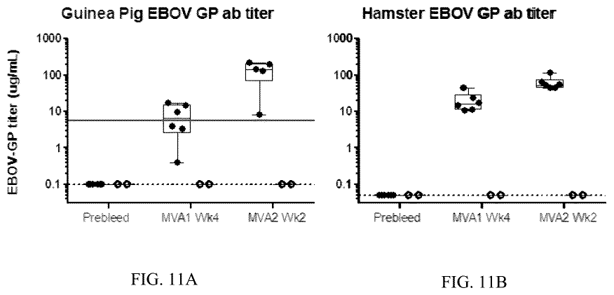

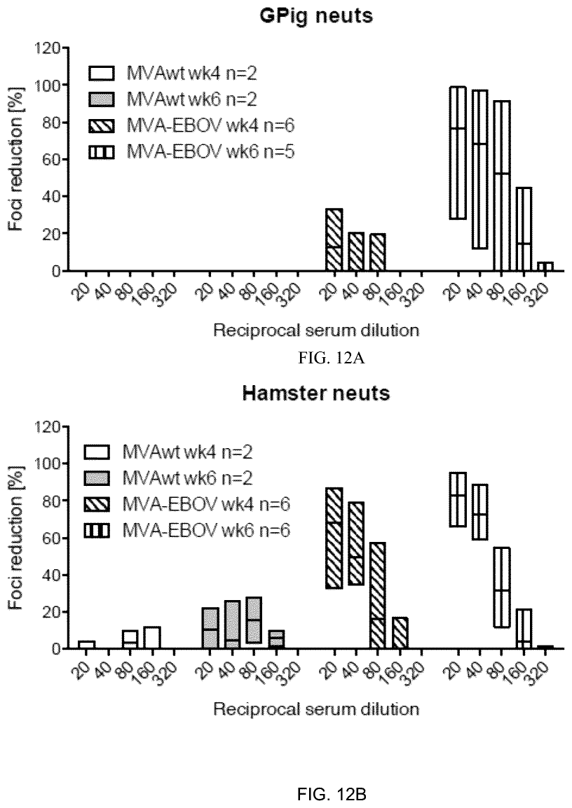

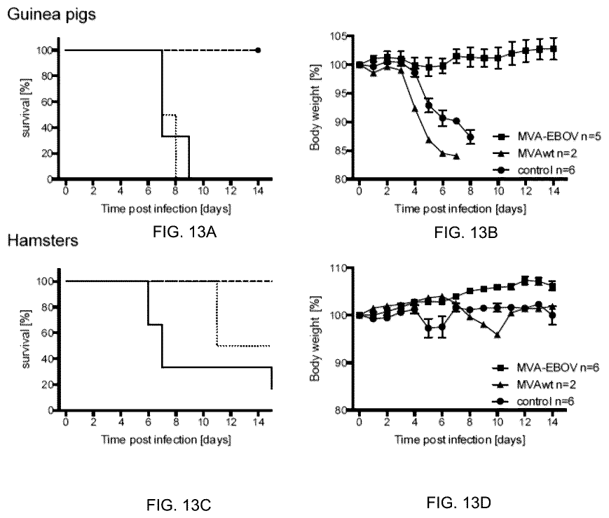

The design for vectors containing EBOV, SUDV, BDBV, MARV and LASV genes is highly similar; therefore, the diagram in FIG. 1 may apply to the recombinant MVA vaccine vectors described in this application. The “GP” annotation in FIG. 1 indicates a GP sequence from EBOV, SUDV, BDBV, or MARV. The “VP40” annotation in FIG. 1 indicates a VP40 sequence from EBOV, SUDV, BDBV, or MARV. Other embodiments may deviate from this general design and are described herein.

In other embodiments for expressing LASV sequences, this illustration may represent a vector expressing LASV sequences where the GP sequence of FIG. 1 may instead represent the Lassa virus GP sequence and the “VP40” sequence of FIG. 1 may instead represent the Lassa virus Z sequence. In another embodiment, the “VP40” in FIG. 1 represents the Lassa virus Z sequence and NP sequence in reverse orientation each operably linked to a promoter compatible with poxvirus expression systems.

FIG. 2 is a schematic for the shuttle vector for filovirus or Marburg virus GP.

The ampicillin resistance marker, allowing the vector to replicate in bacteria, is illustrated with a block labeled “amp-R.” The two flanking sequences, allowing the vector to recombine with the MVA genome, are illustrated with a block and a block labeled “Flank 1” and “Flank 2” respectively. The green fluorescent protein (GFP) selection marker, allowing the selection of recombinant MVAs, is illustrated with an arrow labeled “GFP.” The block labeled “DR” illustrates the location of a sequence homologous to part of Flank 1 of the MVA sequence. DR enables removal of the GFP sequence from the MVA vector after insertion of GP into the MVA genome. The modified H5 (mH5) promoter, which enables transcription of the inserted heterologous gene, is illustrated with a triangle between the DR and GP elements. The filovirus GP gene is illustrated with a white arrow labeled “GP.”

The shuttle vectors for EBOV, SUDV, BDBV, MARV and LASV glycoproteins use a highly similar design; therefore, FIG. 2 provides a single diagram that applies universally to the MVA vaccine vectors described in this application. FIG. 2 illustrates the design of all glycoprotein shuttle vectors of the invention. The “GP” annotation in FIG. 2 applies to glycoprotein sequences from EBOV, SUDV, BDBV, MARV and LASV.

The shuttle vectors for the various species differ in two principal ways. First, the glycoprotein sequences vary by species. Second, the restriction sites used to insert the glycoprotein sequences into the shuttle vector may vary by species. Neither of these differences affects the orientation of the elements of the shuttle vector.

FIG. 3 is a schematic for the shuttle vector for filovirus or Marburg virus VP40.

The ampicillin resistance marker, allowing the vector to replicate in bacteria, is illustrated with a block labeled “amp-R.” The two flanking sequences, allowing the vector to recombine with the MVA genome, are illustrated with blocks labeled “Flank 1” and “Flank 2.” The green fluorescent protein (GFP) selection marker, allowing the selection of recombinant MVAs, is illustrated with an arrow labeled “GFP.” The block labeled “DR” illustrates the location of a sequence homologous to part of Flank 1 of the MVA sequence. DR enables removal of the GFP sequence from the MVA vector after insertion of VP40 into the MVA genome. The modified H5 (mH5) promoter, which enables transcription of the inserted heterologous gene, is illustrated with a triangle between the DR and VP40 elements. The filovirus VP40 gene is illustrated with a white arrow labeled “VP40.”

The shuttle vectors for EBOV, SUDV, BDBV, and MARV VP40s use a highly similar design and naming convention; therefore, FIG. 3 provides a single diagram that applies universally to the MVA vaccine vectors described in this application. FIG. 3 illustrates the design of all VP40 shuttle vectors of the invention. The “VP40” annotation in FIG. 3 applies to VP40 sequences from EBOV, SUDV, BDBV, and MARV.

The shuttle vectors for the various species differ in two principal ways. First, the VP40 sequences vary by species. Second, the restriction sites used to insert the VP40 sequences into the shuttle vector may vary by species. Neither of these differences affects the orientation of the elements of the shuttle vector.

FIG. 4A and FIG. 4B provide a schematic for the shuttle vector for Lassa GP(4A) and Z (4B) genes. The ampicillin resistance marker, allowing the vector to replicate in bacteria, is illustrated with a block labeled “amp-R.” The two flanking sequences, allowing the vector to recombine with the MVA genome, are illustrated with blocks labeled “Flank 1” and “Flank 2.” The green fluorescent protein (GFP) selection marker, allowing the selection of recombinant MVAs, is illustrated with an arrow labeled “GFP.” The block labeled “DR” illustrates the location of a sequence homologous to part of Flank 1 of the MVA sequence. DR enables removal of the GFP sequence from the MVA vector after insertion of GP and Z into the MVA genome. The modified H5 (mH5) promoter and P7.5 promoter, which enable transcription of the inserted heterologous gene, GP and Z respectively, are illustrated with a triangle between the DR and GP or Z elements.

FIG. 5 is an electron micrograph showing virus-like particle (VLP) production by cells transfected with plasmid DNA vectors encoding EBOV GP and VP40 proteins. The sequences of the GP and VP40 in these plasmid DNA vectors are identical to the sequences of the GP and VP40 genes that are used in the MVA vaccine vector that expresses GP and VP40 from the 2014 strain of EBOV. This experiment demonstrated that the 2014 EBOV antigen sequences of this invention are capable of forming VLPs when introduced into cultured cells.

FIG. 6 is a higher magnification of the VLP in FIG. 5 to show the display of ebolavirus GP spikes on the VLP.

FIG. 7 is a schematic for the shuttle vector for pGEO.SUD.VP40.

FIG. 8 is a schematic for the shuttle vector for pGEO.SUD.GP.

FIG. 9 is a schematic for the shuttle vector for pGEO.MAR.VP40.

FIG. 10 is a schematic for the shuttle vector for pGEO.MAR.GP.

FIG. 11A and FIG. 11B show binding antibody responses to the Ebola virus glycoprotein (GP) elicited by the vaccinations and specifically the results for binding Ab elicited by the MVA/Z-VLP vaccine. Guinea pig sera are shown on the left (FIG. 11A) and Syrian golden hamster sera on the right (FIG. 11B). The closed symbols are for animals receiving MVA/Z-VLP vaccine and the open symbols for animals vaccinated with the parental MVA (no vaccine inserts). The horizontal line in the left panel indicates the titer of binding Ab in sera pooled from six guinea pigs vaccinated with a chimeric vesicular stomatitis virus (VSV) expressing GP. Prebleed is prior to first MVA inoculation; MVA1wk4, four weeks after the first MVA inoculation and MVA2wk2, two weeks after the second MVA inoculation.

FIG. 12A and FIG. 12B shows neutralizing Ab responses to Ebola virus elicited by the vaccinations and specifically shows the results for neutralizing Ab elicited by the MVA/Z-VLP vaccine. The upper panel (FIG. 12A), GPig shows neutralizing titers elicited in guinea pigs and the bottom panel (FIG. 12B) shows neutralizing titers elicited in SGH. MVAwt are data for animals infected with parental MVA. MVA-EBOV are data for animals vaccinated with MVA/Z-VLP.

FIG. 13A-FIG. 13B show post challenge survival (left panel FIG. 13A) and body weight charts (right panel FIG. 13B) for guinea pig. FIG. 13C-FIG. 13D show presents post challenge survival (left panel FIG. 13C) and body weight charts (right panel FIG. 13D) for Syrian golden hamster (SGHs). Vaccination with MVA/Z-VLP clearly demonstrates protection against a highly virulent challenge. All of the vaccinated guinea pigs and SGHs survived the challenge.

### DETAILED DESCRIPTION OF THE INVENTION

Compositions and methods are provided to produce an immune response to a hemorrhagic fever virus, such as a member of the genus Ebolavirus, Marburgvirus, or Arenavirus, in a subject in need thereof. The compositions and methods of the present invention can be used to prevent infection in an unexposed person or to treat disease in a subject exposed to a hemorrhagic fever virus who is not yet symptomatic or has minimal symptoms. In one embodiment, treatment limits an infection and/or the severity of disease.

Ideal immunogenic compositions or vaccines have the characteristics of safety, efficacy, scope of protection and longevity, however, compositions having fewer than all of these characteristics may still be useful in preventing viral infection or limiting symptoms or disease progression in an exposed subject treated prior to the development of symptoms. In one embodiment the present invention provides a vaccine that permits at least partial, if not complete, protection after a single immunization.

In one embodiment, the composition is a recombinant vaccine that comprises one or more genes from a hemorrhagic fever virus selected from the group consisting of EBOV, SUDV, BDBV, TAFV, MARV, LASV, and combinations thereof.

In exemplary embodiments, the immune responses are long-lasting and durable so that repeated boosters are not required, but in one embodiment, one or more administrations of the compositions provided herein are provided to boost the initial primed immune response.

### Definitions

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a peptide” includes a plurality of peptides. Thus, for example, a reference to “a method” includes one or more methods, and/or steps of the type described herein and/or which will become apparent to those persons skilled in the art upon reading this disclosure.

The term “antigen” refers to a substance or molecule, such as a protein, or fragment thereof, that is capable of inducing an immune response.

The term “arenavirus” refers to any virus that is a member of the family Arenaviridae.

The term “binding antibody” or “bAb” refers to an antibody which either is purified from, or is present in, a body fluid (e.g., serum or a mucosal secretion) and which recognizes a specific antigen. As used herein, the antibody can be a single antibody or a plurality of antibodies. Binding antibodies comprise neutralizing and non-neutralizing antibodies.

The term “Bundibugyo virus” or “BDBV” refers to a virus belonging to species Bundibugyo ebolavirus.

The term “ ”cell-mediated immune response” refers to the immunological defense provided by lymphocytes, such as the defense provided by sensitized T cell lymphocytes when they directly lyse cells expressing foreign antigens and secrete cytokines (e.g., IFN-gamma.), which can modulate macrophage and natural killer (NK) cell effector functions and augment T cell expansion and differentiation. The cellular immune response is the 2^{nd} branch of the adaptive immune response.

The term “conservative amino acid substitution” refers to substitution of a native amino acid residue with a non-native residue such that there is little or no effect on the size, polarity, charge, hydrophobicity, or hydrophilicity of the amino acid residue at that position, and without resulting in substantially altered immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

The term “deletion” in the context of a polypeptide or protein refers to removal of codons for one or more amino acid residues from the polypeptide or protein sequence. The term deletion in the context of a nucleic acid refers to removal of one or more bases from a nucleic acid sequence.

The term “Ebola virus” or “EBOV” refers to a virus belonging to species Zaire ebolavirus.

The term “Ebolavirus” refers to the genus of the family Filoviridae, order Mononegavirales, which includes the five known species: Zaire ebolavirus, Sudan ebolavirus, Tai Forest ebolavirus (also known as Ivory Coast ebolavirus or Cote d'Ivoire ebolavirus (CIEBOV)), Bundibugyo ebolavirus, and Reston ebolavirus.

The term “ebolavirus” or “Ebolavirus” refers to any member of the genus Ebolavirus.

The term “filovirus” refers collectively to members of the Filoviridae family of single stranded (−) RNA viruses including ebolaviruses and Marburg viruses.

The term “fragment” in the context of a proteinaceous agent refers to a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a peptide, polypeptide or protein. In one embodiment, a fragment of a full-length protein retains activity of the full-length protein. In another embodiment, the fragment of the full-length protein does not retain the activity of the full-length protein.

The term “fragment” in the context of a nucleic acid refers to a nucleic acid comprising an nucleic acid sequence of at least 2 contiguous nucleotides, at least 5 contiguous nucleotides, at least 10 contiguous nucleotides, at least 15 contiguous nucleotides, at least 20 contiguous nucleotides, at least 25 contiguous nucleotides, at least 30 contiguous nucleotides, at least 35 contiguous nucleotides, at least 40 contiguous nucleotides, at least 50 contiguous nucleotides, at least 60 contiguous nucleotides, at least 70 contiguous nucleotides, at least contiguous 80 nucleotides, at least 90 contiguous nucleotides, at least 100 contiguous nucleotides, at least 125 contiguous nucleotides, at least 150 contiguous nucleotides, at least 175 contiguous nucleotides, at least 200 contiguous nucleotides, at least 250 contiguous nucleotides, at least 300 contiguous nucleotides, at least 350 contiguous nucleotides, or at least 380 contiguous nucleotides of the nucleic acid sequence encoding a peptide, polypeptide or protein. In a preferred embodiment, a fragment of a nucleic acid encodes a peptide or polypeptide that retains activity of the full-length protein. In another embodiment, the fragment encodes a peptide or polypeptide that of the full-length protein does not retain the activity of the full-length protein.

As used herein, the term “GP” refers to the ebolavirus or marburgivirus surface glycoprotein, or the gene or transcript encoding the ebolavirus or marburgvirus surface glycoprotein.

As used herein, the phrase “heterologous sequence” refers to any nucleic acid, protein, polypeptide or peptide sequence which is not normally associated in nature with another nucleic acid or protein, polypeptide or peptide sequence of interest.

As used herein, the phrase “heterologous gene insert” refers to any nucleic acid sequence that has been, or is to be inserted into the recombinant vectors described herein. The heterologous gene insert may refer to only the gene product encoding sequence or may refer to a sequence comprising a promoter, a gene product encoding sequence (such as GP, VP or Z), and any regulatory sequences associated or operably linked therewith.

The term “homopolymer stretch” refers to a sequence comprising at least four of the same nucleotides uninterrupted by any other nucleotide, e.g., GGGG or TTTTTTT.

The term “humoral immune response” refers to the stimulation of Ab production. Humoral immune response also refers to the accessory proteins and events that accompany antibody production, including T helper cell activation and cytokine production, affinity maturation, and memory cell generation. The humoral immune response is one of two branches of the adaptive immune response.

The term “humoral immunity” refers to the immunological defense provided by antibody, such as neutralizing Ab that can directly block infection; or, binding Ab that identifies a virus or infected cell for killing by such innate immune responses as complement (C′)-mediated lysis, phagocytosis, and natural killer cells.

The term “immune response” refers to any response to an antigen or antigenic determinant by the immune system of a subject (e.g., a human). Exemplary immune responses include humoral immune responses (e.g., production of antigen-specific antibodies) and cell-mediated immune responses (e.g., production of antigen-specific T cells).

The term “improved therapeutic outcome” relative to a subject diagnosed as infected with a particular virus (e.g., an ebolavirus) refers to a slowing or diminution in the growth of virus, or viral load, or detectable symptoms associated with infection by that particular virus; or a reduction in the ability of the infected subject to transmit the infection to another, uninfected subject.

The term “inducing an immune response” means eliciting a humoral response (e.g., the production of antibodies) or a cellular response (e.g., the activation of T cells) directed against a virus (e.g., ebolavirus) in a subject to which the composition (e.g., a vaccine) has been administered.

The term “insertion” in the context of a polypeptide or protein refers to the addition of one or more non-native amino acid residues in the polypeptide or protein sequence. Typically, no more than about from 1 to 6 residues (e.g. 1 to 4 residues) are inserted at any one site within the polypeptide or protein molecule.

The term “lassavirus,” “Lassa virus,” or “LASV” refers to an arenavirus that is any member of the species Lassa virus.

The term “marburgvirus” or “Marburgvirus” refers to a filovirus that is any member of the genus Marburgvirus.

The term “modified vaccinia Ankara,” “modified vaccinia ankara,” “Modified Vaccinia Ankara,” or “MVA” refers to a highly attenuated strain of vaccinia virus developed by Dr. Anton Mayr by serial passage on chick embryo fibroblast cells; or variants or derivatives thereof. MVA is reviewed in (Mayr, A. et al. 1975 Infection 3:6-14; Swiss Patent No. 568,392).

The term “neutralizing antibody” or “NAb” is meant an antibody which either is purified from, or is present in, a body fluid (e.g., serum or a mucosal secretion) and which recognizes a specific antigen and inhibits the effect(s) of the antigen in the subject (e.g., a human). As used herein, the antibody can be a single antibody or a plurality of antibodies.

The term “non-neutralizing antibody” or “nnAb” refers to a binding antibody that is not a neutralizing antibody.

The term “prevent”, “preventing” and “prevention” refers to the inhibition of the development or onset of a condition (e.g., an ebolavirus infection or a condition associated therewith), or the prevention of the recurrence, onset, or development of one or more symptoms of a condition in a subject resulting from the administration of a therapy or the administration of a combination of therapies.

The term “prophylactically effective amount” refers to the amount of a composition (e.g., the recombinant MVA vector or pharmaceutical composition) which is sufficient to result in the prevention of the development, recurrence, or onset of a condition or a symptom thereof (e.g., an ebolavirus infection or a condition or symptom associated therewith or to enhance or improve the prophylactic effect(s) of another therapy.

The term “recombinant” means a polynucleotide of semisynthetic, or synthetic origin that either does not occur in nature or is linked to another polynucleotide in an arrangement not found in nature.

The term “recombinant,” with respect to a viral vector, means a vector (e.g., a viral genome that has been manipulated in vitro, e.g., using recombinant nucleic acid techniques to express heterologous viral nucleic acid sequences.

The term “regulatory sequence” “regulatory sequences” refers collectively to promoter sequences, polyadenylation signals, transcription termination sequences, upstream regulatory domains, origins of replication, internal ribosome entry sites (“IRES”), enhancers, and the like, which collectively provide for the transcription and translation of a coding sequence. Not all of these control sequences need always be present so long as the selected gene is capable of being transcribed and translated.

The term “shuttle vector” refers to a genetic vector (e.g., a DNA plasmid) that is useful for transferring genetic material from one host system into another. A shuttle vector can replicate alone (without the presence of any other vector) in at least one host (e.g., E. coli). In the context of MVA vector construction, shuttle vectors are usually DNA plasmids that can be manipulated in E. coli and then introduced into cultured cells infected with MVA vectors, resulting in the generation of new recombinant MVA vectors.

The term “silent mutation” means a change in a nucleotide sequence that does not cause a change in the primary structure of the protein encoded by the nucleotide sequence, e.g., a change from AAA (encoding lysine) to AAG (also encoding lysine).

The term “subject” is means any mammal, including but not limited to, humans, domestic and farm animals, and zoo, sports, or pet animals, such as dogs, horses, cats, cows, rats, mice, guinea pigs and the like.

The term “Sudan virus” or SUDV refers to a virus belonging to species Sudan ebolavirus.

The term “surrogate endpoint” means a clinical measurement other than a measurement of clinical benefit that is used as a substitute for a measurement of clinical benefit.

The term “surrogate marker” means a laboratory measurement or physical sign that is used in a clinical or animal trial as a substitute for a clinically meaningful endpoint that is a direct measure of how a subject feels, functions, or survives and is expected to predict the effect of the therapy (Katz, R., NeuroRx 1:189-195 (2004); New drug, antibiotic, and biological drug product regulations; accelerated approval—FDA. Final rule. Fed Regist 57: 58942-58960, 1992.)

The term “surrogate marker for protection” means a surrogate marker that is used in a clinical or animal trial as a substitute for the clinically meaningful endpoint of prevention of ebolavirus or marburgvirus infection.

The term “synonymous codon” refers to the use of a codon with a different nucleic acid sequence to encode the same amino acid, e.g., AAA and AAG (both of which encode lysine). Codon optimization changes the codons for a protein to the synonymous codons that are most frequently used by a vector or a host cell.

The term “Taï forest virus” or “TAFV” refers to a virus belonging to species nil forest ebolavirus.

The term “therapeutically effective amount” means the amount of the composition (e.g., the recombinant MVA vector or pharmaceutical composition) that, when administered to a mammal for treating an infection, is sufficient to effect such treatment for the infection.

The term “treating” or “treat” refer to the eradication or control of a filovirus, a reduction in the titer of the filovirus, a reduction in the numbers of the filovirus, the reduction or amelioration of the progression, severity, and/or duration of a condition or one or more symptoms caused by the filovirus resulting from the administration of one or more therapies, or the reduction or elimination of the subject's ability to transmit the infection to another, uninfected subject.

The term “vaccine” means material used to provoke an immune response and confer immunity after administration of the material to a subject. Such immunity may include a cellular or humoral immune response that occurs when the subject is exposed to the immunogen after vaccine administration.

The term “vaccine insert” refers to a nucleic acid sequence encoding a heterologous sequence that is operably linked to a promoter for expression when inserted into a recombinant vector. The heterologous sequence may encode a glycoprotein or matrix protein described here.

The term “viral infection” means an infection by a viral pathogen (e.g., a member of genus Ebolavirus) wherein there is clinical evidence of the infection based on symptoms or based on the demonstration of the presence of the viral pathogen in a biological sample from the subject.

The term “virus-like particles” or “VLP” refers to a structure which resembles the native virus antigenically and morphologically.

The term “VP40” refers to the ebolavirus or marburgvirus large matrix protein, or the gene or transcript encoding the ebolavirus or marburgvirus large matrix protein.

### II. Filoviruses

The compositions of the present invention are useful for inducing an immune response to a filovirus. The Filoviridae family includes genera Marburgvirus, Ebolavirus and Cuevavirus. Filoviruses are enveloped, negative strand RNA viruses having a thread-like appearance.

Members of genera Ebolavirus and Marburgvirus are among the most pathogenic viruses in humans and non-human primates (Feldman and Klenk, 1996, Adv. Virus Res. 47, 1), both causing severe hemorrhagic fever (HF) (Johnson et al., 1997, Lancet 1, no. 8011, P. 569).

Both are zoonotic agents, where human outbreaks initially occur as a result of direct contact with infected wildlife, with subsequent person-to-person transmission through contact with bodily fluids. Although the ecology of these agents remains incompletely understood, several species of African fruit bats may be reservoirs for members of genera Ebolavirus and Marburgvirus. Filovirus outbreaks are sporadic, sometimes interspersed by years or even decades of no apparent disease activity.

A. Ebolavirus Species and Sequences

The term Ebolavirus refers to a genus within the family Filoviridae. Like other filoviruses, species within the Ebolavirus genus consist of a single strand of negative sense RNA that is approximately 19 kb in length. The RNA contains seven sequentially arranged genes that produce 8 mRNAs upon infection. Ebolavirus virions, like virions of other filoviruses, contain seven proteins: (1) a surface glycoprotein (GP), (2) a nucleoprotein (NP), (3-6) four virion structural proteins (VP40, VP35, VP30, and VP24), and an (7) RNA-dependent RNA polymerase (L). The glycoprotein of an ebolavirus is unlike other filoviruses in that it is encoded in two open reading frames. Transcriptional editing is needed to express the transmembrane form that is incorporated into the virion. The unedited form produces a nonstructural secreted glycoprotein (sGP) that is synthesized in large amounts early during the course of infection.

Based on nucleotide sequence and outbreak location, isolates in genus Ebolavirus are classified into five antigenically distinct species: Zaire ebolavirus, Sudan ebolavirus, nil Forest ebolavirus (also known as Ivory Coast ebolavirus or Cote d'Ivoire ebolavirus (CIEBOV)), Bundibugyo ebolavirus, and Reston ebolavirus. Known viruses belonging to species Zaire ebolavirus are commonly referred to as Ebola viruses(EBOV). Known viruses belonging to species Sudan ebolavirus are commonly referred to as Sudan viruses(SUDV). Known viruses belonging to species Taï Forest ebolavirus are commonly referred to as Tai Forest viruses (TAFV). Known viruses belonging to species Bundibugyo ebolavirus are commonly referred to as Bundibugyo viruses (BDBV). Known viruses belonging to species Marburg marburgvirus include Marburg virus (MARV) and Ravn virus (RAVV).

Of these, EBOV and SUDV are the most pathogenic, and are the only two that have been associated with recurring outbreaks. Together, EBOV and SUDV account for 94% of EBOV-related deaths.

Infection by a member of genus Ebolavirus can lead to Ebola Hemorrhagic Fever (EHF), also known as Ebola Virus Disease (EVD) the clinical manifestations of which are severe. The incubation period varies between 2 to 21 days after exposure to the virus, but the average is 8 to 10 days. The different species in genus Ebolavirus are believed to cause somewhat different clinical syndromes. Even within a single species, variation among strains can cause differences in clinical symptoms. However, opportunities for close observation of the diseases under good conditions have been rare.

The initial symptoms of EHF are generally a severe frontal and temporal headache, generalized aches and pains, malaise, and by the second day the victim will often have a fever. The subsequent signs and symptoms indicate multisystem involvement and include systemic (prostration), gastrointestinal (anorexia, nausea, vomiting, abdominal pain, diarrhea), respiratory (chest pain, shortness of breath, cough, nasal discharge), vascular (conjunctival injection, postural hypotension, oedema) and neurological (headache, confusion, coma) manifestations. Hemorrhagic manifestations arise during the peak of the illness and include petechiae, ecchymoses, uncontrolled oozing from venipuncture sites, mucosal hemorrhages, and post-mortem evidence of visceral hemorrhagic effusions. A macropapular rash associated with varying severity of erythema and desquamate can often be noted by day 5-7 of the illness; this symptom is a valuable differential diagnostic feature and is usually followed by desquamation in survivors. Abdominal pain is sometimes associated with hyperamylasaemia and true pancreatitis. In later stages, shock, convulsions, severe metabolic disturbances, and, in more than half the cases, diffuse coagulopathy supervene. See Sanchez A, Geisbert T W, Feldmann H. Filoviridae: Marburg and Ebola viruses. In: Knipe D M, Howley P M, eds. Fields virology. Philadelphia: Lippincott Williams & Wilkins, 2006: 1409-1448; Pattyn S R. Ebola virus haemorrhagic fever. Amsterdam: Elsevier, North-Holland, 1978; Peters C J, LeDuc L W. Ebola: the virus and the disease. J Infect Dis 1999; 179 (suppl 1): S1-S288; Feldmann H, Geisbert T, Kawaoka Y. Filoviruses: recent advances and future challenges. J Infect Dis 2007; 196 (suppl 2): S129-S443.

Patients with fatal disease develop clinical signs early during infection and typically die between day 6 and 16 as a result of hypovolaemic shock and multiorgan failure. Hemorrhages can be severe but are only present in fewer than half of patients. In non-fatal cases, patients typically have a fever for several days and improve around day 6-11, about the time that the humoral antibody response is noted. Patients with non-fatal or asymptomatic disease mount specific IgM and IgG responses that seem to be associated with a temporary early and strong inflammatory response, including interleukin β, interleukin 6, and tumour necrosis factor α (TNFα).

While case fatality rates vary between outbreaks and among the Ebolavirus species, Zaire ebolavirus has been associated with up to 90% mortality, while Sudan ebolavirus has been associated with up to 60% mortality.

Using current methodology, ebolavirus is detectable in blood only after onset of symptoms, which accompany the rise in circulating virus. It may take up to three days after symptoms start for the virus to reach detectable levels. Laboratory tests used in diagnosis include, for example, antigen-capture enzyme-linked immunosorbent assay (ELISA) testing, IgM ELISA, polymerase chain reaction (PCR), virus isolation, and later in the course of infection or recovery-detection of IgM and IgG antibodies.

No vaccine or therapeutic has been approved by the FDA for ebolavirus, for either prophylactic or therapeutic use. Present treatment strategies are primarily symptomatic and supportive. In developing countries, these strategies typically include isolation, malaria treatment, broad spectrum antibiotics, and antipyretics before diagnosis. Fluid substitution, preferentially intravenous administration, and analgesics may also be provided. In developed countries with facilities having appropriate isolation units, intensive care treatment is provided and directed towards maintenance of effective blood volume and electrolyte balance. Shock, cerebral edema, renal failure, coagulation disorders, and secondary bacterial infection must also be managed. Organ failure is also addressed, e.g., by dialysis for kidney failure and extracorporeal membrane oxygenation for lung failure.

B. Marburg Virus Species and Sequences

Marburgviruses are substantially identical structurally to ebolaviruses. The marburgvirus genome consists of a single strand of negative sense RNA that is approximately 19.1 kb in length and which encodes a series of polypeptides that correspond in sequence and function to those of ebolaviruses, although the exact intergenic regions are different between the two genera. Thus, a marburgvirus consists of seven polypeptides, which are (as in ebolaviruses) the envelope glycoprotein (GP), the nucleoprotein (NP), matrix proteins VP24 and VP40, the transcription factor VP30, the polymerase cofactor VP35, and the viral polymerase.

Only one species of marburgvirus has been reported, Marburg marburgvirus (formerly Lake Victoria marburgvirus), and two individual viruses, Marburg virus (MARV) and Ravn virus (RAVN), within this species.

Marburg hemorrhagic fever (MHF) may affects both humans and non-human primates. After an incubation period of 5-10 days, the onset of the disease is sudden and is marked by fever, chills, headache, and myalgia. Around the fifth day after the onset of symptoms, a maculopapular rash, most prominent on the trunk (chest, back, stomach), may occur. Nausea, vomiting, chest pain, a sore throat, abdominal pain, and diarrhea may then appear. Symptoms become increasingly severe and may include jaundice, inflammation of the pancreas, severe weight loss, delirium, shock, liver failure, massive hemorrhaging, and multi-organ dysfunction.

There is no vaccine for marburgvirus approved by the FDA, either prophylactic or therapeutic. As with EHF, current treatment generally currently consists of supportive therapy, including maintenance of blood volume and electrolyte balance, as well as analgesics and standard nursing care.

C. Lassa Virus Species and Sequences

Lassa virus is an arenavirus belonging to genus Arenavirus, family Arenaviridae. The arenavirus genome consists of two single-stranded negative-sense RNAs, one approximately 7.2 kb in length and the other approximately 3.5 kb in length. Each of the RNAs encodes two proteins. The gene sequences for the proteins are oriented in opposite directions; this arrangement is referred to as an ambisense coding strategy. The large (7.2 kb) genomic RNA encodes the RNA-dependent RNA polymerase (L) protein and the matrix (Z) protein. The small (3.5 kb) genomic RNA encodes the nucleoprotein (NP) and the glycoprotein precursor (GP). On each genomic RNA, the two genes are separated by an intergenic region (IGR) The ambisense coding strategy results in different mechanisms of transcription for the four proteins. The NP and L mRNAs are transcribed directly from the genomic RNA. The GP and Z mRNAs, on the other hand, are translated from anti-genomic RNAs. The IGR is believed to serve as a signal for termination of transcription. (Shao et al. (2015), Pathogens 4: 283-306).

Lassa fever is the acute hemorrhagic fever caused by Lassa virus. Symptoms typically appear 6-21 days after infection. Approximately 80% of cases are mild, involving mild fever, general malaise, weakness, and headache. In approximately 20% of cases, Lassa fever causes more severe symptoms including high fever, sore throat, mucosal bleeding, respiratory distress, vomiting, swelling, severe pain, and shock. Certain neurological problems may also occur. Of patients hospitalized for Lassa fever, approximately 15%-20% die from the infection (Kyei et al. (2015), BMC Infectious Diseases 15:217). Unlike filoviruses, which cause sporadic outbreaks, Lassa virus is a common human pathogen that causes endemic disease in a large area of West Africa (Andersen et al. (2015), Cell 162:738-750). Official estimates indicate 300,000-500,000 cases of Lassa fever each year with approximately 5,000-10,000 deaths; however, other measures indicate that the disease may be much more serious, accounting for as many as 3 million cases and 67,000 deaths annually (Leski et al. (2015) Emerging Infectious Diseases 21(4):609-618). Several experimental vaccines against LASV have been tested in animal models. To date, however, no Lassa fever vaccine has yet been approved for sale (Falzarano and Feldmann (2015), Current Opinion in Virology 3:343-351). Other than supportive care, there are few options for treatment of Lassa virus infection. Only the broad-spectrum antiviral drug ribavirin has shown efficacy, and it must be used early in the course of the disease in order to be effective (Olschlager and Flatz (2013), PLoS Pathogens 9(4):e1003212).

### III. Recombinant Viral Vectors

In one aspect, the present invention is a recombinant viral vector comprising one or more genes of a hemorrhagic fever virus, such as an Ebolavirus, a Marburgvirus, or an Arenavirus. In certain embodiments, the recombinant viral vector is a vaccinia viral vector, and more particularly, an MVA vector, comprising one or more genes of a hemorrhagic fever virus, such as an Ebolavirus, a Marburgvirus, or an Arenavirus.

Vaccinia viruses have also been used to engineer viral vectors for recombinant gene expression and for the potential use as recombinant live vaccines (Mackett, M. et al 1982 PNAS USA 79:7415-7419; Smith, G. L. et al. 1984 Biotech Genet Engin Rev 2:383-407). This entails DNA sequences (genes) which code for foreign antigens being introduced, with the aid of DNA recombination techniques, into the genome of the vaccinia viruses. If the gene is integrated at a site in the viral DNA which is non-essential for the life cycle of the virus, it is possible for the newly produced recombinant vaccinia virus to be infectious, that is to say able to infect foreign cells and thus to express the integrated DNA sequence (EP Patent Applications No. 83,286 and No. 110,385). The recombinant vaccinia viruses prepared in this way can be used, on the one hand, as live vaccines for the prophylaxis of infectious diseases, on the other hand, for the preparation of heterologous proteins in eukaryotic cells.

Several such strains of vaccinia virus have been developed to avoid undesired side effects of smallpox vaccination. Thus, a modified vaccinia Ankara (MVA) has been generated by long-term serial passages of the Ankara strain of vaccinia virus (CVA) on chicken embryo fibroblasts (for review see Mayr, A. et al. 1975 Infection 3:6-14; Swiss Patent No. 568,392). The MVA virus is publicly available from American Type Culture Collection as ATCC No.: VR-1508. MVA is distinguished by its great attenuation, as demonstrated by diminished virulence and reduced ability to replicate in primate cells, while maintaining good immunogenicity. The MVA virus has been analyzed to determine alterations in the genome relative to the parental CVA strain. Six major deletions of genomic DNA (deletion I, II, III, IV, V, and VI) totaling 31,000 base pairs have been identified (Meyer, H. et al. 1991 J Gen Virol 72:1031-1038). The resulting MVA virus became severely host cell restricted to avian cells.

Furthermore, MVA is characterized by its extreme attenuation. When tested in a variety of animal models, MVA was proven to be avirulent even in immunosuppressed animals. More importantly, the excellent properties of the MVA strain have been demonstrated in extensive clinical trials (Mayr A. et al. 1978 Zentralbl Bakteriol [B] 167:375-390; Stickl et al. 1974 Dtsch Med Wschr 99:2386-2392). During these studies in over 120,000 humans, including high-risk patients, no side effects were associated with the use of MVA vaccine.

MVA replication in human cells was found to be blocked late in infection preventing the assembly to mature infectious virions. Nevertheless, MVA was able to express viral and recombinant genes at high levels even in non-permissive cells and was proposed to serve as an efficient and exceptionally safe gene expression vector (Sutter, G. and Moss, B. 1992 PNAS USA 89:10847-10851). Additionally, novel vaccinia vector vaccines were established on the basis of MVA having foreign DNA sequences inserted at the site of deletion III within the MVA genome (Sutter, G. et al. 1994 Vaccine 12:1032-1040).

Recombinant MVA vaccinia viruses can be prepared as set out hereinafter. A DNA-construct which contains a DNA-sequence which codes for a foreign polypeptide flanked by MVA DNA sequences adjacent to a predetermined insertion site (e.g. between two conserved essential MVA genes such as I8R/G1L; in restructured and modified deletion III; or at other non-essential sites within the MVA genome) is introduced into cells infected with MVA, to allow homologous recombination. Once the DNA-construct has been introduced into the eukaryotic cell and the foreign DNA has recombined with the viral DNA, it is possible to isolate the desired recombinant vaccinia virus in a manner known per se, preferably with the aid of a marker. The DNA-construct to be inserted can be linear or circular. A plasmid or polymerase chain reaction product is preferred. Such methods of making recombinant MVA vectors are described in PCT publication WO/2006/026667 incorporated by reference herein. The DNA-construct contains sequences flanking the left and the right side of a naturally occurring deletion. The foreign DNA sequence is inserted between the sequences flanking the naturally occurring deletion. For the expression of a DNA sequence or gene, it is necessary for regulatory sequences, which are required for the transcription of the gene, to be present on the DNA. Such regulatory sequences (called promoters) are known to those skilled in the art, and include for example those of the vaccinia 11 kDa gene as are described in EP-A-198,328, and those of the 7.5 kDa gene (EP-A-110,385). The DNA-construct can be introduced into the MVA infected cells by transfection, for example by means of calcium phosphate precipitation (Graham et al. 1973 Virol 52:456-467; Wigler et al. 1979 Cell 16:777-785), by means of electroporation (Neumann et al. 1982 EMBO J. 1:841-845), by microinjection (Graessmann et al. 1983 Meth Enzymol 101:482-492), by means of liposomes (Straubinger et al. 1983 Meth Enzymol 101:512-527), by means of spheroplasts (Schaffher 1980 PNAS USA 77:2163-2167) or by other methods known to those skilled in the art.

The MVA vectors described and tested herein were unexpectedly found to be effective after a single prime or a homologous prime/boost regimen. Other MVA vector designs require a heterologous prime/boost regimen while still other published studies have been unable to induce effective immune responses with MVA vectors. Conversely, the present MVA vector design and methods of manufacture are useful in producing effective MVA vaccine vectors for eliciting effective T-cell and antibody immune responses. Furthermore, the utility of an MVA vaccine vector capable of eliciting effective immune responses and antibody production after a single homologous prime boost is significant for considerations such as use, commercialization and transport of materials especially to affected third world locations.

In one embodiment, the present invention is a recombinant viral vector (e.g., an MVA vector) comprising one or more heterologous gene inserts of a filovirus (e.g., an ebolavirus or marburgvirus). The viral vector (e.g., an MVA vector) may be constructed using conventional techniques known to one of skill in the art. The one or more heterologous gene inserts encode a polypeptide having desired immunogenicity, i.e., a polypeptide that can induce an immune reaction, cellular immunity and/or humoral immunity, in vivo by administration thereof. The gene region of the viral vector (e.g., an MVA vector) where the gene encoding a polypeptide having immunogenicity is introduced is flanked by regions that are indispensable. In the introduction of a gene encoding a polypeptide having immunogenicity, an appropriate promoter may be operatively linked upstream of the gene encoding a polypeptide having desired immunogenicity.

The one or more genes may be selected from any species of hemorrhagic fever virus. In one embodiment, the one more genes are selected from an Ebolavirus, Marburgvirus or Arenavirus species, and more particularly, a hemorrhagic fever virus selected from the group consisting of EBOV, SUDV, TAFV, BDBV, RESTV, MARV, and LASV, or a combination thereof. In exemplary embodiments, the gene encodes a polypeptide or protein capable of inducing an immune response in the subject to which it is administered, and more particularly, an immune response capable of providing a protective and/or therapeutic benefit to the subject. In one embodiment, the one or more genes encode the virus glycoprotein (GP), the secreted GP (sGP), the major nucleoprotein (NP), RNA-dependent RNA polymerase (L), or one or more virion structural proteins (e.g., Z, VP40, VP35, VP30, or VP24)). The heterologous gene inserts are inserted into one or more deletion sites of the vector under the control of promoters compatible with poxvirus expression systems.

In one embodiment, the deletion III site is restructured and modified to remove non-essential flanking sequences.

In exemplary embodiments, the vaccine is constructed to express an ebolavirus GP for example EBOV GP, which is inserted between two conserved essential MVA genes (I8R and G1L) using shuttle vector pGeo-GP; and to express EBOV VP40, which is inserted into deletion III using shuttle vector pGeo-VP40. pGeo-GP and pGeo-VP40 are constructed with an ampicillin resistance marker, allowing the vector to replicate in bacteria; with two flanking sequences, allowing the vector to recombine with a specific location in the MVA genome; with a green fluorescent protein (GFP) selection marker, allowing the selection of recombinant MVAs; with a sequence homologous to part of Flank 1 of the MVA sequence, enabling removal of the GFP sequence from the MVA vector after insertion of VP40 into the MVA genome; with a modified H5 (mH5) promoter, which enables transcription of the inserted heterologous gene insert; and with a filovirus gene. pGeo-GP and pGeo-VP40 differ in that pGeo-GP contains the GP sequence, whereas pGeo-VP40 contains the VP40 sequence; and in that pGeo-GP recombines with sequences of MVA I8R and G1L (two essential genes) and pGeo-VP40 recombines with regions flanking the restructured and modified Deletion III of MVA.

In exemplary embodiments, the present invention provides a recombinant MVA vector comprising a gene encoding the glycoprotein (GP) gene and a gene encoding VP40, in each case, from an ebolavirus, marburgvirus, or Lassa virus.

In certain embodiments, the polypeptide, or the nucleic acid sequence encoding the polypeptide, may have a mutation or deletion (e.g., an internal deletion, truncation of the amino- or carboxy-terminus, or a point mutation).

The one or more genes introduced into the recombinant viral vector are under the control of regulatory sequences that direct its expression in a cell.

The nucleic acid material of the viral vector may be encapsulated, e.g., in a lipid membrane or by structural proteins (e.g., capsid proteins), that may include one or more viral polypeptides.

In exemplary embodiments, the present invention is a recombinant viral vector (e.g., a recombinant MVA vector) comprising one or more genes, or one or more polypeptides encoded by the gene or genes, from an ebolavirus, marburgvirus, or Lassa virus. The ebolavirus, marburgvirus, or Lassa virus gene may encode a polypeptide or protein capable of inducing an immune response in the subject to which it is administered, and more particularly, an immune response capable of providing a protective and/or therapeutic benefit to the subject, e.g., the ebolavirus, marburgvirus, or Lassa virus glycoprotein. As used herein, the term “ebolavirus, marburgvirus, or Lassa virus glycoprotein” refers to the glycoprotein polypeptide encoded by the ebolavirus, marburgvirus, or Lassa virus genome, whether in secreted or transmembrane bound form, or any fragment or mutation of the glycoprotein polypeptide, that is encoded by the ebolavirus, marburgvirus, or Lassa virus genome so long as it has the ability to induce or enhance an immune response or confer a protective or therapeutic benefit to the subject, e.g., against one or more of SUDV, EBOV, TAFV, BDBV, MARV, or LASV. The nucleic acid sequences of ebolavirus, marburgvirus, or Lassa virus glycoproteins are published and are available from a variety of sources, including, e.g., GenBank and PubMed. Exemplary GenBank references including ebolavirus, marburgvirus, or Lassa virus glycoprotein sequences include those corresponding to accession numbers KM233103 (EBOV, 2014 strain), KC242798 (EBOV, central sequence), KC545390 (SUDV), KC545396 (BDBV), NC 001608 (MARV), and JN650517 (LASV GP and NP) and JN650518 (LASV Z).

In certain embodiments, the one or more genes encodes a polypeptide, or fragment thereof, that is substantially identical (e.g., at least 60%, 65%, 70%, 75%, 80%, 85%, 90%, 95%, 98%, 99%, or even 100% identical) to the selected ebolavirus, marburgvirus, or Lassa virus glycoprotein over at least 20, 25, 30, 35, 40, 45, 50, 55, 60, 65, or 70 contiguous residues of the selected ebolavirus, marburgvirus, or Lassa virus glycoprotein that retain immunogenic activity.

In exemplary embodiments, the recombinant viral vector may also include an ebolavirus, marburgvirus, or Lassa virus glycoprotein present on its surface. The ebolavirus, marburgvirus, or Lassa virus glycoprotein may be obtained by any suitable means, including, e.g., application of genetic engineering techniques to a viral source, chemical synthesis techniques, recombinant production, or any combination thereof.

In another embodiments, the present invention is a recombinant MVA vector comprising at least one heterologous gene insert from an ebolavirus, marburgvirus, or Lassa virus, wherein the gene is selected from the group encoding the glycoprotein (GP), the secreted GP (sGP), the major nucleoprotein (NP), RNA-dependent RNA polymerase (L), or one or more other viral proteins (e.g., Z, VP40, VP35, VP30, or VP24)).

In a particular embodiment, the present invention is a recombinant MVA vector comprising a gene encoding GP and a gene encoding VP40. In another embodiment, the present invention is a recombinant MVA vector comprising genes encoding GP, Z, and NP. The heterologous gene inserts are inserted into one or more deletion sites of the MVA vector under the control of promoters compatible with poxvirus expression systems.

In one embodiment, the GP is inserted into deletion site I, II, III, IV, V or VI of the MVA vector, and the VP40 is inserted into deletion site I, II, III, IV, V or VI of the MVA vector.

In one embodiment, the GP is inserted between I8R and G1L of the MVA vector, or into restructured and modified deletion III of the MVA vector; and the VP40 is inserted between I8R and GIL of the MVA vector, or into restructured and modified deletion site III of the MVA vector.

In one embodiment relating to LASV, the GP is inserted into deletion site I, II, III, IV, V or VI of the MVA vector, and the Z is inserted into deletion site I, II, III, IV, V or VI of the MVA vector.

In one embodiment, the recombinant vector comprises in a first deletion site, a gene encoding GP operably linked to a promoter compatible with poxvirus expression systems, and in a second deletion site, genes encoding Z and NP in reverse orientation each operably linked to a promoter compatible with poxvirus expression systems.

In one embodiment relating to LASV, the GP is inserted between I8R and GIL of the MVA vector, or into restructured and modified deletion III of the MVA vector; and the Z is inserted between I8R and G1L of the MVA vector, or into restructured and modified deletion site III of the MVA vector.

In another embodiment relating to LASV, the GP and Z are inserted into different deletion sites. For example, the GP sequence is inserted between two essential and highly conserved MVA genes, I8R/G1L, to limit the formation of viable deletion mutants; and, the Z sequence is inserted into a restructured and modified deletion III site.

In exemplary embodiments, the present invention is a recombinant MVA vector comprising at least one heterologous gene insert (e.g., one or more gene inserts) from an ebolavirus or a marburgvirus which is under the control of regulatory sequences that direct its expression in a cell. The gene may be, for example, under the control of a promoter selected from the group consisting of Pm2H5, Psyn II, or mH5 promoters.

The recombinant viral vector of the present invention can be used to infect cells of a subject, which, in turn, promotes the translation into a protein product of the one or more viral genes of the viral vector (e.g., an ebolavirus, marburgvirus, or Lassa virus glycoprotein). As discussed further herein, the recombinant viral vector can be administered to a subject so that it infects one or more cells of the subject, which then promotes expression of the one or more viral genes of the viral vector and stimulates an immune response that is protective against infection by an ebolavirus, marburgvirus, or Lassa virus (e.g., EBOV) or that reduces or prevents infection by an ebolavirus, marburgvirus, or Lassa virus (e.g., EBOV).

In one embodiment, the recombinant MVA vaccine expresses proteins that assemble into virus-like particles (VLPs) comprising the GP (glycoprotein), and VP40 (matrix protein). While not wanting to be bound by any particular theory, it is believed that the GP is provided to elicit a protective immune response and the VP40 (matrix protein) is provided to enable assembly of VLPs and as a target for T cell immune responses, thereby enhancing the protective immune response and providing cross-protection.

Similarly relating to LASV, in one embodiment, the recombinant MVA vaccine expresses proteins that assemble into virus-like particles (VLPs) comprising the GP (glycoprotein), and Z (matrix protein). While not wanting to be bound by any particular theory, it is believed that the GP is provided to elicit a protective immune response and the Z (matrix protein) is provided to enable assembly of VLPs and as a target for T cell immune responses, thereby enhancing the protective immune response and providing cross-protection.

For references, see Stahelin, Front in Microbiol 5:300 (2014); Marzi et al., J Infect Dis 204 Suppl 3:S1066 (2011); Warfield and Aman, J Infect Dis 204 Suppl 3:S1053 (2011); and Mire et al., PLoS Negl Trop Dis 7:e2600 (2013).

One or more genes may be optimized for use in an MVA vector. Optimization includes codon optimization, which employs silent mutations to change selected codons from the native sequences into synonymous codons that are optimally expressed by the host-vector system. Other types of optimization include the use of silent mutations to interrupt homopolymer stretches or transcription terminator motifs. Each of these optimization strategies can improve the stability of the gene, improve the stability of the transcript, or improve the level of protein expression from the gene. In exemplary embodiments, the number of homopolymer stretches in the GP or VP40 sequence will be reduced to stabilize the construct. A silent mutation may be provided for anything similar to a vaccinia termination signal. An extra nucleotide may be added in order to express the transmembrane, rather than the secreted, form of ebolavirus GP.

In exemplary embodiments, the GP and VP40 sequences are codon optimized for expression in MVA using a computer algorithm; GP and VP40 sequences with runs of ≥5 deoxyguanosines, ≥5 deoxycytidines, ≥5 deoxyadenosines, and ≥5 deoxythymidines are interrupted by silent mutation to minimize loss of expression due to frame shift mutations; and the GP sequence is modified through addition of an extra nucleotide to express the transmembrane, rather than the secreted, form of ebolavirus GP.

In one embodiment, the present invention provides a vaccine vector composition that is monovalent. As used herein the term monovalent refers to a vaccine vector composition that contains GP and matrix sequences from one species of ebolavirus, Marbugvirus, or Arenavirus.

In another embodiment, the present invention provides a vaccine that is bivalent. As used herein the term monovalent refers to a vaccine vector composition that contains two vectors having GP and matrix sequences from different species of ebolavirus, Marbugvirus, or Arenavirus.

In another embodiment, the present invention provides a vaccine that is trivalent. As used herein the term trivalent refers to a vaccine vector composition that contains three vectors having GP and matrix sequences from different species of ebolavirus, Marbugvirus, or Arenavirus.

In another embodiment, the present invention provides a vaccine that is quadrivalent. As used herein the term quadrivalent refers to a vaccine vector composition that contains four vectors having GP and matrix sequences from different species of ebolavirus, Marbugvirus, or Arenavirus. As used herein, the terms tetravalent and quadrivalent are synonymous.

In one embodiment, the recombinant viral vector (e.g., an MVA vector) comprises two heterologous gene inserts from an Ebolavirus species, a Marbugvirus species, or an Arenavirus species, wherein the first heterologous gene insert and the second heterologous gene insert are from the same species of Ebolavirus, Marbugvirus, or Arenavirus species.

In another embodiment, the recombinant viral vector (e.g., an MVA vector) comprises two heterologous gene inserts from an Ebolavirus species, a Marbugvirus species, or an Arenavirus species, wherein the first heterologous gene insert is from an Ebolavirus, Marbugvirus, or Arenavirus species different than the second heterologous gene insert. In one embodiment, the first heterologous gene insert is from the EBOV virus and the second heterologous gene insert is from an ebolavirus or a marburgvirus selected from SUDV, TAFV, BDBV, RESTV, MARV, or LASV.

In exemplary embodiments, the recombinant viral vector (e.g., an MVA vector) comprises three heterologous gene inserts from an Ebolavirus species, or a Marbugvirus species, or an Arenavirus species, wherein the first heterologous gene insert is from an Ebolavirus species, a Marbugvirus species, or an Arenavirus species different at least from one of the second or third heterologous gene inserts. In one embodiment, the first heterologous gene insert is from the EBOV virus and the second and third heterologous gene inserts are selected from an ebolavirus or a marburgvirus selected from SUDV, TAFV, BDBV, RESTV, MARV, or LASV. The second and third heterologous gene inserts may be the same or different.

The recombinant viral vectors of the present invention may be used alone, or in combination. In one embodiment, two different recombinant viral vectors are used in combination, where the difference may refer to the one or more heterologous gene inserts or the other components of the recombinant viral vector or both. In exemplary embodiments, two or more recombinant viral vectors are used in combination in order to protect against infection by all versions of ebolavirus, marburgvirus, and Lassa virus known to be lethal in humans.

The present invention also extends to host cells comprising the recombinant viral vector described above, as well as isolated virions prepared from host cells infected with the recombinant viral vector.

### IV. Pharmaceutical Composition

The recombinant viral vectors of the present invention are readily formulated as pharmaceutical compositions for veterinary or human use, either alone or in combination. The pharmaceutical composition may comprise a pharmaceutically acceptable diluent, excipient, carrier, or adjuvant.

In one embodiment, the present invention is a vaccine effective to protect and/or treat a hemorrhagic fever virus (e.g., an ebolavirus) comprising a recombinant MVA vector that expresses at least one hemorrhagic fever virus polypeptide (e.g., a GP) or an immunogenic fragment thereof. The vaccine composition may comprise one or more additional therapeutic agents.

The pharmaceutical composition may comprise 1, 2, 3, 4 or more than 4 different recombinant MVA vectors.

In one embodiment, the present invention provides a vaccine vector composition that is monovalent. As used herein the term monovalent refers to a vaccine vector composition that contains GP and matrix sequences from one species of ebolavirus, Marbugvirus, or Arenavirus.

In another embodiment, the present invention provides a vaccine that is bivalent. As used herein the term monovalent refers to a vaccine vector composition that contains two vectors having GP and matrix sequences from different species of ebolavirus, Marbugvirus, or Arenavirus.

In another embodiment, the present invention provides a vaccine that is trivalent. As used herein the term trivalent refers to a vaccine vector composition that contains three vectors having GP and matrix sequences from different species of ebolavirus, Marbugvirus, or Arenavirus.

In another embodiment, the present invention provides a vaccine that is quadrivalent. As used herein the term monovalent refers to a vaccine vector composition that contains four vectors having GP and matrix sequences from different species of ebolavirus, Marbugvirus, or Arenavirus. As used herein, the terms tetravalent and quadrivalent are synonymous.

As used herein, the phrase “pharmaceutically acceptable carrier” encompasses any of the standard pharmaceutical carriers, such as those suitable for parenteral administration, such as, for example, by intramuscular, intraarticular (in the joints), intravenous, intradermal, intraperitoneal, and subcutaneous routes. Examples of such formulations include aqueous and non-aqueous, isotonic sterile injection solutions, which contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. One exemplary pharmaceutically acceptable carrier is physiological saline.

Other physiologically acceptable diluents, excipients, carriers, or adjuvants and their formulations are known to those skilled in the art.

The compositions utilized in the methods described herein can be administered by a route selected from, e.g., parenteral, intramuscular, intraarterial, intravascular, intravenous, intraperitoneal, subcutaneous, dermal, transdermal, ocular, inhalation, buccal, sublingual, perilingual, nasal, topical administration, and oral administration. The preferred method of administration can vary depending on various factors (e.g., the components of the composition being administered and the severity of the condition being treated). Formulations suitable for oral administration may consist of liquid solutions, such as an effective amount of the composition dissolved in a diluent (e.g., water, saline, or PEG-400), capsules, sachets or tablets, each containing a predetermined amount of the vaccine. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen).

For the purposes of this invention, pharmaceutical compositions suitable for delivering a therapeutic or biologically active agent can include, e.g., tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21.sup.st ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

The immunogenicity of the composition (e.g., vaccine) may be significantly improved if the composition of the present invention is co-administered with an immunostimulatory agent or adjuvant. Suitable adjuvants well-known to those skilled in the art include, e.g., aluminum phosphate, aluminum hydroxide, QS21, Quil A (and derivatives and components thereof), calcium phosphate, calcium hydroxide, zinc hydroxide, glycolipid analogs, octodecyl esters of an amino acid, muramyl dipeptides, polyphosphazene, lipoproteins, ISCOM-Matrix, DC-Chol, DDA, cytokines, and other adjuvants and derivatives thereof.

Pharmaceutical compositions according to the invention described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

Formulations suitable for oral administration can consist of (a) liquid solutions, such as an effective amount of the vaccine dissolved in diluents, such as water, saline or PEG 400; (b) capsules, sachets or tablets, each containing a predetermined amount of the vaccine, as liquids, solids, granules or gelatin; (c) suspensions in an appropriate liquid; (d) suitable emulsions; and (e) polysaccharide polymers such as chitins. The vaccine, alone or in combination with other suitable components, may also be made into aerosol formulations to be administered via inhalation, e.g., to the bronchial passageways. Aerosol formulations can be placed into pressurized acceptable propellants, such as dichlorodifluoromethane, propane, nitrogen, and the like.

Suitable formulations for rectal administration include, for example, suppositories, which consist of the vaccine with a suppository base. Suitable suppository bases include natural or synthetic triglycerides or paraffin hydrocarbons. In addition, it is also possible to use gelatin rectal capsules which consist of a combination of the vaccine with a base, including, for example, liquid triglycerides, polyethylene glycols, and paraffin hydrocarbons.

Pharmaceutical compositions comprising any of the nucleic acid molecules encoding Ebola viral proteins of the present invention are useful to immunize a subject against disease caused by ebolavirus infection. Thus, this invention further provides methods of immunizing a subject against disease caused by ebolavirus infection, e.g., hemorrhagic fever, comprising administering to the subject an immunoeffective amount of a pharmaceutical composition of the invention. This subject may be an animal, for example a mammal, such as a primate or preferably a human.

The vaccines of the present invention are also suitable for veterinary immunization. The vaccines of the present invention comprising nucleic acid molecules encoding ebolavirus structural gene products from the Reston ebolavirus species, which is known to infect animals, are particularly useful in such veterinary immunization methods.

The vaccines of the present invention may also be co-administered with cytokines to further enhance immunogenicity. The cytokines may be administered by methods known to those skilled in the art, e.g., as a nucleic acid molecule in plasmid form or as a protein or fusion protein.

### Kits

This invention also provides kits comprising the vaccines of the present invention. For example, kits comprising a vaccine and instructions for use are within the scope of this invention.

### V. Method of Use

The compositions of the invention can be used as vaccines for inducing an immune response to a filovirus or an arenavirus, such as a member of the genus Ebolavirus, the genus Marburgvirus, or the genus Arenavirus, including any species thereof.

In exemplary embodiments, the present invention provides a method of preventing a filovirus or arenavirus (e.g., ebolavirus) infection to a subject in need thereof (e.g., an unexposed) subject, said method comprising administering the composition of the present invention to the subject in a prophylactically effective amount. The result of the method is that the subject is partially or completely immunized against the virus.

In exemplary embodiments, the present invention provides a method of treating a filovirus or arenavirus (e.g., ebolavirus) infection in a subject in need thereof (e.g., an exposed subject, such as a subject who has been recently exposed but is not yet symptomatic, or a subject who has been recently exposed and is only mildly symptomatic), said method comprising administering the composition of the present invention to the subject in a therapeutically effective amount. The result of treatment is a subject that has an improved therapeutic profile.

In certain embodiments, the compositions of the invention can be used as vaccines for treating a subject infected with more than one filovirus or more than one areavirus, e.g., multiple species of Ebolavirus or Arenavirus. The recombinant viral vector comprises genes or sequences encoding viral proteins of multiple species of Ebolavirus or Arenavirus and/or the pharmaceutical composition comprises more than one type of recombinant viral vector, in terms of the heterologous gene inserts or sequences contained.

Typically the vaccines will be in an admixture and administered simultaneously, but may also be administered separately.

A subject to be treated according to the methods described herein (e.g., a subject infected with, an ebolavirus) may be one who has been diagnosed by a medical practitioner as having such a condition. Diagnosis may be performed by any suitable means. A subject in whom the development of an infection is being prevented may or may not have received such a diagnosis. One skilled in the art will understand that a subject to be treated according to the present invention may have been identified using standard tests or may have been identified, without examination, as one at high risk due to the presence of one or more risk factors (e.g., exposure to ebolavirus, etc.).

Prophylactic treatment may be administered, for example, to a subject not yet exposed to or infected by a hemorrhagic fever virus but who is susceptible to, or otherwise at risk of exposure or infection with an a hemorrhagic fever virus.

Therapeutic treatment may be administered, for example, to a subject already exposed to or infected by a hemorrhagic fever virus who is not yet ill, or showing symptoms or infection, suffering from a disorder in order to improve or stabilize the subject's condition (e.g., a patient already infected with an a hemorrhagic fever virus). The result is an improved therapeutic profile. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique. In some instances, treating can result in the inhibition of viral replication, a decrease in viral titers or viral load, eradication or clearing of the virus.

In other embodiments, treatment may result in amelioration of one or more symptoms of the infection, including any symptom identified above. According to this embodiment, confirmation of treatment can be assessed by detecting an improvement in or the absence of symptoms.

In other embodiments, treatment may result in reduction or elimination of the ability of the subject to transmit the infection to another, uninfected subject. Confirmation of treatment according to this embodiment is generally assessed using the same methods used to determine amelioration of the disorder, but the reduction in viral titer or viral load necessary to prevent transmission may differ from the reduction in viral titer or viral load necessary to ameliorate the disorder.

In one embodiment, the present invention is a method of inducing an immune response in a subject (e.g., a human) by administering to the subject a recombinant viral vector that encodes at least one gene from a hemorrhagic fever virus, such as a member of genus Ebolavirus a member of genus Marburgvirus, or a member of genus Arenavirus. The immune response may be a cellular immune response or a humoral immune response, or a combination thereof.

In a particular embodiment, the present invention is a method of inducing an immune response in a subject (e.g., a human) by administering to the subject a recombinant viral vector that encodes at least one gene from a member of genus Ebolavirus, more particularly, EBOV. In certain embodiments, the recombinant viral vector encodes at least two genes from an ebolavirus, more particularly, EBOV. The immune response may be a cellular immune response or a humoral immune response, or a combination thereof.

In another particular embodiment, the present invention is a method of inducing an immune response in a subject (e.g., a human) by administering to the subject a recombinant viral vector that encodes at that encodes at least one gene from a member of genus Marburgvirus, more particularly, MARV. In certain embodiments, the recombinant viral vector encodes at least two genes from a marburgvirus, more particularly, MARV. The immune response may be a cellular immune response or a humoral immune response, or a combination thereof.

In a particular embodiment, the present invention is a method of inducing an immune response in a subject (e.g., a human) by administering to the subject a recombinant viral vector that encodes at least one gene from a member of genus Ebolavirus, more particularly, SUDV. In certain embodiments, the recombinant viral vector encodes at least two genes from an ebolavirus, more particularly, SUDV. The immune response may be a cellular immune response or a humoral immune response, or a combination thereof.

In a particular embodiment, the present invention is a method of inducing an immune response in a subject (e.g., a human) by administering to the subject a recombinant viral vector that encodes at least one gene from a member of genus Ebolavirus, more particularly, BDBV. In certain embodiments, the recombinant viral vector encodes at least two genes from an ebolavirus, more particularly, BDBV. The immune response may be a cellular immune response or a humoral immune response, or a combination thereof.

In a particular embodiment, the present invention is a method of inducing an immune response in a subject (e.g., a human) by administering to the subject a recombinant viral vector that encodes at least one gene from a member of genus Arenavirus, more particularly, LASV. In certain embodiments, the recombinant viral vector encodes at least two genes from an arenavirus, more particularly, LASV. The immune response may be a cellular immune response or a humoral immune response, or a combination thereof.

In another embodiment, the invention features a method of treating a filovirus infection (e.g., an ebolavirus infection) in a subject (e.g., a human) in need thereof by administering to the subject a recombinant viral vector that encodes at least one gene from the Zaire ebolavirus species of ebolavirus (e.g., the EBOV glycoprotein). The subject being treated may not have, but is at risk of developing, an infection by a filovirus, for example, an infection caused by a filovirus selected from TAFV, EBOV, SUDV, BDBV, MARV or a combination thereof.

In another embodiment, the invention features a method of treating a filovirus infection (e.g., an ebolavirus infection) in a subject (e.g., a human) by administering to the subject a recombinant viral vector that encodes at least one gene from the Sudan ebolavirus species of ebolavirus (e.g., the SUDV glycoprotein). The subject being treated may not have, but is at risk of developing, an infection by a filovirus, for example, an infection caused by a filovirus selected from TAFV, EBOV, SUDV, BDBV, MARV or a combination thereof.

In another embodiment, the invention features a method of treating a filovirus infection (e.g., an ebolavirus infection) in a subject (e.g., a human) by administering to the subject a recombinant viral vector that encodes at least one gene from the Bundibugyo ebolavirus species of ebolavirus (e.g., the BDBV glycoprotein). The subject being treated may not have, but is at risk of developing, an infection by a filovirus, for example, an infection caused by a filovirus selected from TAFV, EBOV, SUDV, BDBV, MARV or a combination thereof.

In another embodiment, the invention features a method of treating a filovirus infection (e.g., a marburgvirus infection) in a subject (e.g., a human) by administering to the subject a recombinant viral vector that encodes at least one gene from the Marburg marburgvirus species of marburgvirus (e.g., the MARV glycoprotein). The subject being treated may not have, but is at risk of developing, an infection by a filovirus, for example, an infection caused by a filovirus selected from TAFV, EBOV, SUDV, BDBV, MARV or a combination thereof.

In another embodiment, the invention features a method of treating an arenavirus infection (e.g., a Lassa virus infection) in a subject (e.g., a human) by administering to the subject a recombinant viral vector that encodes at least one gene from the Lassa virus species of arenavirus (e.g., the LASV glycoprotein). The subject being treated may not have, but is at risk of developing, an infection by an arenavirus, for example, an infection caused by LASV.

In another embodiment, the subject may already be infected with at least one filovirus or arenavirus (e.g., an ebolavirus or a Lassa virus). The infection may be caused by a hemorrhagic fever virus selected from the group consisting of TAFV, EBOV, SUDV, BDBV, MARV, LASV, or a combination thereof.

The composition may be administered, e.g., by injection (e.g., intramuscular, intraarterial, intravascular, intravenous, intraperitoneal, or subcutaneous).

It will be appreciated that more than one route of administering the vaccines of the present invention may be employed either simultaneously or sequentially (e.g., boosting). In addition, the vaccines of the present invention may be employed in combination with traditional immunization approaches such as employing protein antigens, vaccinia virus and inactivated virus, as vaccines. Thus, in one embodiment, the vaccines of the present invention are administered to a subject (the subject is “primed” with a vaccine of the present invention) and then a traditional vaccine is administered (the subject is “boosted” with a traditional vaccine). In another embodiment, a traditional vaccine is first administered to the subject followed by administration of a vaccine of the present invention. In yet another embodiment, a traditional vaccine and a vaccine of the present invention are co-administered.

While not to be bound by any specific mechanism, it is believed that upon inoculation with a pharmaceutical composition as described herein, the immune system of the host responds to the vaccine by producing antibodies, both secretory and serum, specific for ebolavirus, marburgvirus, or Lassa virus proteins; and by producing a cell-mediated immune response specific for ebolavirus, marburgvirus, or Lassa virus. As a result of the vaccination, the host becomes at least partially or completely immune to ebolavirus, marburgvirus, or Lassa virus infection, or resistant to developing moderate or severe disease caused by ebolavirus, marburgvirus, or Lassa virus infection.

In one aspect, methods are provided to alleviate, reduce the severity of, or reduce the occurrence of, one or more of the symptoms (e.g., fever, hemorrhagic fever, severe headache, muscle pain, malaise, extreme asthenia, conjunctivitis, popular rash, dysphagia, nausea, vomiting, bloody diarrhea followed by diffuse hemorrhages, delirium, shock, jaundice, thrombocytopenia, lymphocytopenia, neutrophilia, focal necrosis in various organs (e.g., kidneys and liver), and acute respiratory distress) associated with ebolavirus, marburgvirus, or Lassa virus infection comprising administering an effective amount of a pharmaceutical composition comprising a recombinant MVA viral vector that comprises GP and VP40 sequences from the Zaire ebolavirus, Sudan ebolavirus, Taï Forest ebolavirus, Bundibugyo ebolavirus, Reston ebolavirus, or Marburg marburgvirus species of filovirus; or comprising GP and Z sequences from the Lassa virus species of arenavirus; or comprising GP, Z, and NP sequences from the Lassa virus species of arenavirus.

In one embodiment, the MVA viral vector comprises GP and VP40 sequences from a Zaire ebolavirus species.

In one embodiment, the MVA viral vector comprises GP and VP40 sequences from a Sudan ebolavirus species.

In one embodiment, the MVA viral vector comprises GP and VP40 sequences from aBundibugyo ebolavirus species.

In one embodiment, the MVA viral vector comprises GP and VP40 sequences from a Marburg marburgvirus species.

In one embodiment, the MVA viral vector comprises GP and Z sequences from a Lassa virus species.

In one embodiment, the MVA viral vector comprises GP, Z, and NP sequences from a Lassa virus species.

In another embodiment, a combination of at least two different recombinant MVA viral vectors are administered wherein the GP and VP40 sequences are from a Zaire ebolavirus, Sudan ebolavirus, Taï Forest ebolavirus, Bundibugyo ebolavirus, Reston ebolavirus, or Marburg marburgvirus species of filovirus. Also included in this embodiment are combinations of one recombinant MVA viral vector encoding GP and VP40 from a filovirus with another recombinant MVA viral vector encoding GP and Z or GP, Z, and NP from the Lassa virus species of arenavirus.

In one embodiment, the pharmaceutical composition comprises two recombinant MVA vaccines expressing GP and VP40 sequences from a Zaire ebolavirus and a Bundibugyo ebolavirus species of ebolavirus.

In one embodiment, the pharmaceutical composition comprises two recombinant MVA vaccines expressing GP and VP40 sequences from a Zaire ebolavirus and a Sudan ebolavirus.

In one embodiment, the pharmaceutical composition comprises two recombinant MVA vaccines expressing GP and VP40 sequences from a Sudan ebolavirus and a Bundibugyo ebolavirus.

In one embodiment, the pharmaceutical composition comprises two recombinant MVA vaccines expressing GP and VP40 sequences from a Zaire ebolavirus and a Marburg marburgvirus.

In one embodiment, the pharmaceutical composition comprises two recombinant MVA vaccines expressing GP and VP40 sequences from a Bundibugyo ebolavirus species and a Marburg marburgvirus.

In one embodiment, the pharmaceutical composition comprises two recombinant MVA vaccines expressing GP and VP40 sequences from a Sudan ebolavirus and a Marburg marburgvirus

In one embodiment, the pharmaceutical composition comprises two recombinant MVA vaccines, one expressing GP and VP40 sequences from a Zaire ebolavirus and the other expressing GP and Z sequences from a Lassa virus.

In one embodiment, the pharmaceutical composition comprises two recombinant MVA vaccines, one expressing GP and VP40 sequences from a Sudan ebolavirus and the other expressing GP and Z sequences from a Lassa virus.

In one embodiment, the pharmaceutical composition comprises two recombinant MVA vaccines, one expressing GP and VP40 sequences from a Bundibugyo ebolavirus and the other expressing GP and Z sequences from a Lassa virus.

In one embodiment, the pharmaceutical composition comprises two recombinant MVA vaccines, one expressing GP and VP40 sequences from a Marburg marburgvirus and the other expressing GP and Z sequences from a Lassa virus.

In one embodiment, the pharmaceutical composition comprises two recombinant MVA vaccines, one expressing GP and VP40 sequences from a Zaire ebolavirus and the other expressing GP, Z, and NP sequences from a Lassa virus.

In one embodiment, the pharmaceutical composition comprises two recombinant MVA vaccines, one expressing GP and VP40 sequences from a Sudan ebolavirus and the other expressing GP, Z, and NP sequences from a Lassa virus.

In one embodiment, the pharmaceutical composition comprises two recombinant MVA vaccines, one expressing GP and VP40 sequences from a Bundibugyo ebolavirus and the other expressing GP, Z, and NP sequences from a Lassa virus.

In one embodiment, the pharmaceutical composition comprises two recombinant MVA vaccines, one expressing GP and VP40 sequences from a Marburg marburgvirus and the other expressing GP, Z, and NP sequences from a Lassa virus.

In another embodiment, a combination of three or more different recombinant MVA viral vectors are administered wherein the GP and VP40 sequences are from a Zaire ebolavirus, a Sudan ebolavirus, a Taï Forest ebolavirus, a Bundibugyo ebolavirus, a Reston ebolavirus, or a Marburg marburgvirus species of Filovirus. Also included in this embodiment are combinations of two or more recombinant MVA viral vectors encoding GP and VP40 from filoviruses with another recombinant MVA viral vector encoding GP and Z or GP, Z, and NP from the Lassa virus species of arenavirus.

In one embodiment, the pharmaceutical composition comprises three recombinant MVA vaccines expressing glycoprotein and matrix protein sequences from a Zaire ebolavirus, a Bundibugyo ebolavirus, and a Sudan ebolavirus.

In one embodiment, the pharmaceutical composition comprises three recombinant MVA vaccines expressing glycoprotein and matrix protein sequences from a Zaire ebolavirus, a Bundibugyo ebolavirus, and a Marburg marburgvirus.

In one embodiment, the pharmaceutical composition comprises three recombinant MVA vaccines expressing glycoprotein and matrix protein sequences from a Zaire ebolavirus, a Sudan ebolavirus, and a Marburg marburgvirus.

In one embodiment, the pharmaceutical composition comprises three recombinant MVA vaccines expressing glycoprotein and matrix protein sequences from a Bundibugyo ebolavirus, a Sudan ebolavirus, and a Marburg marburgvirus.

In one embodiment, the pharmaceutical composition comprises three recombinant MVA vaccines expressing glycoprotein and matrix protein sequences from a Zaire ebolavirus, a Sudan ebolavirus, and a Lassa virus.

In one embodiment, the pharmaceutical composition comprises three recombinant MVA vaccines expressing glycoprotein and matrix protein sequences from a Zaire ebolavirus, a Bundibugyo ebolavirus, and a Lassa virus.

In one embodiment, the pharmaceutical composition comprises three recombinant MVA vaccines expressing glycoprotein and matrix protein sequences from a Zaire ebolavirus, a Marburg marburgvirus, and a Lassa virus.

In one embodiment, the pharmaceutical composition comprises three recombinant MVA vaccines expressing glycoprotein and matrix protein sequences from a Sudan ebolavirus, a Bundibugyo ebolavirus, and a Lassa virus.

In one embodiment, the pharmaceutical composition comprises three recombinant MVA vaccines expressing glycoprotein and matrix protein sequences from a Sudan ebolavirus, a Marburg marburgvirus, and a Lassa virus.

In one embodiment, the pharmaceutical composition comprises three recombinant MVA vaccines expressing glycoprotein and matrix protein sequences from a Bundibugyo ebolavirus, a Marburg marburgvirus, and a Lassa virus.

In one embodiment, the pharmaceutical composition comprises three recombinant MVA vaccines expressing glycoprotein and matrix protein sequences from a Zaire ebolavirus, a Sudan ebolavirus, and a Lassa virus, and the recombinant MVA comprising Lassa virus sequences also expresses the nucleoprotein of a Lassa virus.

In one embodiment, the pharmaceutical composition comprises three recombinant MVA vaccines expressing glycoprotein and matrix protein sequences from a Zaire ebolavirus, a Bundibugyo ebolavirus, and a Lassa virus, and the recombinant MVA comprising Lassa virus sequences also expresses the nucleoprotein of a Lassa virus.

In one embodiment, the pharmaceutical composition comprises three recombinant MVA vaccines expressing glycoprotein and matrix protein sequences from a Zaire ebolavirus, a Marburg marburgvirus, and a Lassa virus, and the recombinant MVA comprising Lassa virus sequences also expresses the nucleoprotein of a Lassa virus.

In one embodiment, the pharmaceutical composition comprises three recombinant MVA vaccines expressing glycoprotein and matrix protein sequences from a Sudan ebolavirus, a Bundibugyo ebolavirus, and a Lassa virus, and the recombinant MVA comprising Lassa virus sequences also expresses the nucleoprotein of a Lassa virus.

In one embodiment, the pharmaceutical composition comprises three recombinant MVA vaccines expressing glycoprotein and matrix protein sequences from a Sudan ebolavirus, a Marburg marburgvirus, and a Lassa virus, and the recombinant MVA comprising Lassa virus sequences also expresses the nucleoprotein of a Lassa virus.

In one embodiment, the pharmaceutical composition comprises three recombinant MVA vaccines expressing glycoprotein and matrix protein sequences from a Bundibugyo ebolavirus, a Marburg marburgvirus, and a Lassa virus, and the recombinant MVA comprising Lassa virus sequences also expresses the nucleoprotein of a Lassa virus.

In one embodiment, the pharmaceutical composition comprises four recombinant MVA vaccines expressing glycoprotein and matrix protein sequences from a Zaire ebolavirus, a Bundibugyo ebolavirus, a Sudan ebolavirus, and a Marburg marburgvirus.

In one embodiment, the pharmaceutical composition comprises four recombinant MVA vaccines expressing glycoprotein and matrix protein sequences from a Zaire ebolavirus, a Bundibugyo ebolavirus, a Sudan ebolavirus, and a Lassa virus.

In one embodiment, the pharmaceutical composition comprises four recombinant MVA vaccines expressing glycoprotein and matrix protein sequences from a Sudan ebolavirus, a Bundibugyo ebolavirus, a Marburg marburgvirus, and a Lassa virus.

In one embodiment, the pharmaceutical composition comprises four recombinant MVA vaccines expressing glycoprotein and matrix protein sequences from a Zaire ebolavirus, a Bundibugyo ebolavirus, a Marburg marburgvirus, and a Lassa virus.

In one embodiment, the pharmaceutical composition comprises four recombinant MVA vaccines expressing glycoprotein and matrix protein sequences from a Zaire ebolavirus, a Bundibugyo ebolavirus, a Sudan ebolavirus, and a Lassa virus, and the recombinant MVA comprising Lassa virus sequences also expresses the nucleoprotein of a Lassa virus.

In one embodiment, the pharmaceutical composition comprises four recombinant MVA vaccines expressing glycoprotein and matrix protein sequences from a Sudan ebolavirus, a Bundibugyo ebolavirus, a Marburg marburgvirus, and a Lassa virus, and the recombinant MVA comprising Lassa virus sequences also expresses the nucleoprotein of a Lassa virus.

In one embodiment, the pharmaceutical composition comprises four recombinant MVA vaccines expressing glycoprotein and matrix protein sequences from a Zaire ebolavirus, a Bundibugyo ebolavirus, a Marburg marburgvirus, and a Lassa virus, and the recombinant MVA comprising Lassa virus sequences also expresses the nucleoprotein of a Lassa virus.

In another aspect, the invention provides methods of inducing an immune response to ebolavirus, marburgvirus, or Lassa virus comprising administering an effective amount of a pharmaceutical composition comprising a recombinant MVA vaccine expressing glycoprotein and matrix protein from at least one species of ebolavirus, marburgvirus, or Lassa virus. The Lassa vaccine of this aspect may also express the Lassa virus nucleoprotein.

In another aspect, the invention provides methods of providing anti-ebolavirus, anti-marburgvirus, or anti-Lassa virus immunity comprising administering an effective amount of a pharmaceutical composition comprising a recombinant MVA vaccine expressing glycoprotein and matrix protein from at least one species of ebolavirus, marburgvirus, or Lassa virus. The Lassa vaccine of this aspect may also express the Lassa virus nucleoprotein.

In another aspect, the invention provides methods of reducing the spread of ebolavirus, marburgvirus, or Lassa virus infection within a subject or from an infected subject to an uninfected subject, comprising administering an effective amount of a pharmaceutical composition comprising a recombinant MVA vaccine expressing glycoprotein and matrix protein from at least one species of ebolavirus, marburgvirus, or Lassa virus. The Lassa vaccine of this aspect may also express the Lassa virus nucleoprotein. In another aspect, the invention provides methods of reducing symptoms of ebolavirus, marburgvirus, or Lassa virus infection comprising administering an effective amount of a pharmaceutical composition comprising a recombinant MVA vaccine expressing glycoprotein and matrix protein from at least one species of ebolavirus, marburgvirus, or Lassa virus. The Lassa vaccine of this aspect may also express the Lassa virus nucleoprotein. In another aspect, the invention provides methods of inducing an immune response which is considered a surrogate marker for protection against ebolavirus, marburgvirus, or Lassa virus infection. Data for determination of whether a response constitutes a surrogate marker for protection are obtained using immune response data obtained using the measurements outlined above.

It will also be appreciated that single or multiple administrations of the vaccine compositions of the present invention may be carried out. For example, subjects who are particularly susceptible to ebolavirus, marburgvirus, or Lassa virus infection may require multiple immunizations to establish and/or maintain protective immune responses. Levels of induced immunity can be monitored by measuring amounts of binding and neutralizing secretory and serum antibodies as well as levels of T cells, and dosages adjusted or vaccinations repeated as necessary to maintain desired levels of protection.

In one embodiment, administration is repeated at least twice, at least 3 times, at least 4 times, at least 5 times, at least 6 times, at least 7 times, at least 8 times, or more than 8 times.

In one embodiment, administration is repeated twice.

In one embodiment, about 2-8, about 4-8, or about 6-8 administrations are provided.

In one embodiment, about 1-4-week, 2-4 week, 3-4 week, 1 week, 2 week, 3 week, 4 week or more than 4 week intervals are provided between administrations.

In one specific embodiment, a 4-week interval is used between 2 administrations.

### Dosage

The vaccines are administered in a manner compatible with the dosage formulation, and in such amount as will be therapeutically effective, immunogenic and protective. The quantity to be administered depends on the subject to be treated, including, for example, the capacity of the immune system of the individual to synthesize antibodies, and, if needed, to produce a cell-mediated immune response. Precise amounts of active ingredient required to be administered depend on the judgment of the practitioner and may be monitored on a patient-by-patient basis. However, suitable dosage ranges are readily determinable by one skilled in the art and generally range from about 5.0×10⁶ TCID₅₀ to about 5.0×10⁹ TCID₅₀. The dosage may also depend, without limitation, on the route of administration, the patient's state of health and weight, and the nature of the formulation.

The pharmaceutical compositions of the invention are administered in such an amount as will be therapeutically effective, immunogenic, and/or protective against a pathogenic species of ebolavirus. The dosage administered depends on the subject to be treated (e.g., the manner of administration and the age, body weight, capacity of the immune system, and general health of the subject being treated). The composition is administered in an amount to provide a sufficient level of expression that elicits an immune response without undue adverse physiological effects. Preferably, the composition of the invention is a heterologous viral vector that includes one or more polypeptides of the ebolavirus, marburgvirus, or Lassa virus (e.g., the ebolavirus, marburgvirus, or Lassa virus glycoprotein and large matrix protein; the Lassa vaccine of this invention may also express the Lassa virus nucleoprotein), or a nucleic acid molecule encoding one or more genes of the ebolavirus, marburgvirus, or Lassa virus, and is administered at a dosage of, e.g., between 1.0×10⁴ and 9.9×10¹² TCID₅₀ of the viral vector, preferably between 1.0×10 5 TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). A physician or researcher can decide the appropriate amount and dosage regimen.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition to the subject two or more times.

The invention also features a method of inducing an immune response to ebolavirus, marburgvirus, or Lassa virus in a subject (e.g., a human) that includes administering to the subject an effective amount of a recombinant viral vector that encodes at least one gene from the ebolavirus (e.g., the ebolavirus, marburgvirus, or Lassa virus glycoprotein and large matrix protein; the Lassa vaccine of this invention may also express the Lassa virus nucleoprotein). The infection may be caused by the Zaire ebolavirus, Sudan ebolavirus, nil Forest ebolavirus, Bundibugyo ebolavirus, or Reston ebolavirus species of ebolavirus; by the Marburg marburgvirus species of marburgvirus; or by the Lassa virus species of arenavirus. The subject being treated may not have, but is at risk of developing, an infection by an ebolavirus, a marburgvirus, or an arenavirus. Alternatively, the subject may already be infected with an ebolavirus, a marburgvirus, or an arenavirus. The composition may be administered, e.g., by injection (e.g., intramuscular, intraarterial, intravascular, intravenous, intraperitoneal, or subcutaneous).

The term “effective amount” is meant the amount of a composition administered to improve, inhibit, or ameliorate a condition of a subject, or a symptom of a disorder, in a clinically relevant manner (e.g., improve, inhibit, or ameliorate infection by ebolavirus, marburgvirus, or arenavirus or provide an effective immune response to infection by ebolavirus, marburgvirus, or arenavirus). Any improvement in the subject is considered sufficient to achieve treatment. Preferably, an amount sufficient to treat is an amount that prevents the occurrence or one or more symptoms of ebolavirus, marburgvirus, or arenavirus infection or is an amount that reduces the severity of, or the length of time during which a subject suffers from, one or more symptoms of ebolavirus, marburgvirus, or arenavirus infection (e.g., by at least 10%, 20%, or 30%, more preferably by at least 50%, 60%, or 70%, and most preferably by at least 80%, 90%, 95%, 99%, or more, relative to a control subject that is not treated with a composition of the invention). A sufficient amount of the pharmaceutical composition used to practice the methods described herein (e.g., the treatment of ebolavirus infection) varies depending upon the manner of administration and the age, body weight, and general health of the subject being treated. Ultimately, the prescribers or researchers will decide the appropriate amount and dosage.

It is important to note that the value of the present invention may never be demonstrated in terms of actual clinical benefit. Instead, it is likely that the value of the invention will be demonstrated in terms of success against a surrogate marker for protection. For an indication such as ebolavirus, marburgvirus, or Lassa virus infection, in which it is impractical or unethical to attempt to measure clinical benefit of an intervention, the FDA's Accelerated Approval process allows approval of a new vaccine based on efficacy against a surrogate endpoint. Therefore, the value of the invention may lie in its ability to induce an immune response that constitutes a surrogate marker for protection.

Similarly, FDA may allow approval of vaccines against ebolaviruses, marburgviruses, or arenaviruses based on its Animal Rule. In this case, approval is achieved based on efficacy in animals. The value of the invention may lie in its ability to protect relevant animal species against infection with ebolaviruses, marburgviruses, or arenaviruses, thus providing adequate evidence to justify its approval.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition two or more times.

In some instances it may be desirable to combine the ebolavirus, marburgvirus, or arenavirus vaccines of the present invention with vaccines which induce protective responses to other agents, particularly other viruses. For example, the vaccine compositions of the present invention can be administered simultaneously, separately or sequentially with other genetic immunization vaccines such as those for influenza (Ulmer, J. B. et al., Science 259:1745-1749 (1993); Raz, E. et al., PNAS (USA) 91:9519-9523 (1994)), malaria (Doolan, D. L. et al., J. Exp. Med. 183:1739-1746 (1996); Sedegah, M. et al., PNAS (USA) 91:9866-9870 (1994)), and tuberculosis (Tascon, R. C. et al., Nat. Med. 2:888-892 (1996)).

### Administration

As used herein, the term “administering” refers to a method of giving a dosage of a pharmaceutical composition of the invention to a subject. The compositions utilized in the methods described herein can be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, intraarterial, intravascular, and intramuscular administration. The preferred method of administration can vary depending on various factors (e.g., the components of the composition being administered and the severity of the condition being treated).

Administration of the pharmaceutical compositions (e.g., vaccines) of the present invention can be by any of the routes known to one of skill in the art. Administration may be by, e.g., intramuscular injection. The compositions utilized in the methods described herein can also be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. The preferred method of administration can vary depending on various factors, e.g., the components of the composition being administered and the severity of the condition being treated.

In addition, single or multiple administrations of the compositions of the present invention may be given to a subject. For example, subjects who are particularly susceptible to ebolavirus infection may require multiple treatments to establish and/or maintain protection against the virus. Levels of induced immunity provided by the pharmaceutical compositions described herein can be monitored by, e.g., measuring amounts of neutralizing secretory and serum antibodies. The dosages may then be adjusted or repeated as necessary to maintain desired levels of protection against viral infection.

The claimed invention is further describe by way of the following non-limiting examples. Further aspects and embodiments of the present invention will be apparent to those of ordinary skill in the art, in view of the above disclosure and following experimental exemplification, included by way of illustration and not limitation, and with reference to the attached figures.

### EXAMPLES

### Example 1. MVA Vaccine Vectors

This Example provides information on exemplary MVA vaccine vectors. Table 1 lists seven MVA vaccine vectors.

TABLE 1
MVA vaccine vectors

| Vaccine | | | |
|---|---|---|---|
| designation | GP sequence | Matrix protein sequence | Nucleoprotein sequence |
| GEO-EM01 | Optimized GP sequence for | Optimized VP40 sequence for | Not applicable |
| | EBOV 2014 (current epidemic) | EBOV 2014 (current epidemic) | |
| GEO-EM02 | Optimized GP sequence for | Optimized VP40 sequence for | Not applicable |
| | EBOV, central EBOV sequence | EBOV, central EBOV sequence | |
| GEO-EM03 | Optimized GP sequence for | Optimized VP40 sequence for | Not applicable |
| | SUDV, central SUDV sequence | SUDV, central SUDV sequence | |
| GEO-EM04 | Optimized GP sequence for | Optimized VP40 sequence for | Not applicable |
| | BDBV, central BDBV sequence | BDBV, central BDBV sequence | |
| GEO-EM05 | Optimized GP sequence for | Optimized VP40 sequence for | Not applicable |
| | MARV, 1980 Mt. Elgon- | MARV, 1980 Mt. Elgon- | |
| | Musoke strain | Musoke strain | |
| GEO-EM06 | Optimized GP sequence for | Optimized Z sequence for | Not applicable |
| | LASV, Josiah strain | LASV, Josiah strain | |
| GEO-EM07 | Optimized GP sequence for | Optimized Z sequence for | Optimized NP sequence for |
| | LASV, Josiah strain | LASV, Josiah strain | LASV, Josiah strain |

Table 2 lists the accession numbers for the GenBank sequences used for design of the five MVA vaccine vectors of this invention

TABLE 2
MVA vaccine vectors of this invention, source of sequences

| Vaccine | GenBank accession number for |
|---|---|
| designation | source sequence |
| GEO-EM01 | KM233103.1 |
| GEO-EM02 | KC242798.1 |
| GEO-EM03 | KC545390.1 |
| GEO-EM04 | KC545396.1 |
| GEO-EM05 | NC_001608 |
| GEO-EM06 | JN650517.1, JN650518.1 |
| GEO-EM07 | JN650517.1, JN650518.1 |

### Example 2. Sequence Optimization

Example 2 illustrates the process for optimization of GP and VP40 sequences for use in an MVA vaccine vector. This Example shows the optimization of one GP and one VP40 sequence, both of which are included in GEO-EM01 (the vaccine for the 2014 EBOV strain). The process followed for vaccines against other strains is highly similar, involving the same set of operations.

The native nucleotide sequence for 2014 EBOV GP (which would lead to expression of sGP) was obtained from GenBank (accession number KM233103.1)

SEQ ID 01:
Native nucleotide sequence for 2014
EBOV GP, from GenBank:
ATGGGTGTTACAGGAATATTGCAGTTACCTCGTGA
TCGATTCAAGAGGACATCATTCTTTCTTTGGGTAA
TTATCCTTTTCCAAAGAACATTTTCCATCCCGCTT
GGAGTTATCCACAATAGTACATTACAGGTTAGTGA
TGTCGACAAACTAGTTTGTCGTGACAAACTGTCAT
CCACAAATCAATTGAGATCAGTTGGACTGAATCTC
GAGGGGAATGGAGTGGCAACTGACGTGCCATCTGT
GACTAAAAGATGGGGCTTCAGGTCCGGTGTCCCAC
CAAAGGTGGTCAATTATGAAGCTGGTGAATGGGCT
GAAAACTGCTACAATCTTGAAATCAAAAAACCTGA
CGGGAGTGAGTGTCTACCAGCAGCGCCAGACGGGA
TTCGGGGCTTCCCCCGGTGCCGGTATGTGCACAAA
GTATCAGGAACGGGACCATGTGCCGGAGACTTTGC
CTTCCACAAAGAGGGTGCTTTCTTCCTGTATGATC
GACTTGCTTCCACAGTTATCTACCGAGGAACGACT
TTCGCTGAAGGTGTCGTTGCATTTCTGATACTGCC
CCAAGCTAAGAAGGACTTCTTCAGCTCACACCCCT
TGAGAGAGCCGGTCAATGCAACGGAGGACCCGTCG
AGTGGCTATTATTCTACCACAATTAGATATCAGGC
TACCGGTTTTGGAACTAATGAGACAGAGTACTTGT
TCGAGGTTGACAATTTGACCTACGTCCAACTTGAA
TCAAGATTCACACCACAGTTTCTGCTCCAGCTGAA
TGAGACAATATATGCAAGTGGGAAGAGGAGCAACA
CCACGGGAAAACTAATTTGGAAGGTCAACCCCGAA
ATTGATACAACAATCGGGGAGTGGGCCTTCTGGGA
AACTAAAAAAACCTCACTAGAAAAATTCGCAGTGA
AGAGTTGTCTTTCACAGCTGTATCAAACGGACCCA
AAAACATCAGTGGTCAGAGTCCGGCGCGAACTTCT
TCCGACCCAGAGACCAACACAACAAATGAAGACCA
CAAAATCATGGCTTCAGAAAATTCCTCTGCAATGG
TTCAAGTGCACAGTCAAGGAAGGAAAGCTGCAGTG
TCGCATCTGACAACCCTTGCCACAATCTCCACGAG
TCCTCAACCTCCCACAACCAAAACAGGTCCGGACA
ACAGCACCCATAATACACCCGTGTATAAACTTGAC
ATCTCTGAGGCAACTCAAGTTGGACAACATCACCG
TAGAGCAGACAACGACAGCACAGCCTCCGACACTC
CCCCCGCCACGACCGCAGCCGGACCCTTAAAAGCA
GAGAACACCAACACGAGTAAGAGCGCTGACTCCCT
GGACCTCGCCACCACGACAAGCCCCCAAAACTACA
GCGAGACTGCTGGCAACAACAACACTCATCACCAA
GATACCGGAGAAGAGAGTGCCAGCAGCGGGAAGCT
AGGCTTAATTACCAATACTATTGCTGGAGTAGCAG
GACTGATCACAGGCGGGAGAAGGACTCGAAGAGAA
GTAATTGTCAATGCTCAACCCAAATGCAACCCCAA
TTTACATTACTGGACTACTCAGGATGAAGGTGCTG
CAATCGGATTGGCCTGGATACCATATTTCGGGCCA
GCAGCCGAAGGAATTTACACAGAGGGGCTAATGCA
CAACCAAGATGGTTTAATCTGTGGGTTGAGGCAGC
TGGCCAACGAAACGACTCAAGCTCTCCAACTGTTC
CTGAGAGCCACAACTGAGCTGCGAACCTTTTCAAT
CCTCAACCGTAAGGCAATTGACTTCCTGCTGCAGC
GATGGGGTGGCACATGCCACATTTTGGGACCGGAC
TGCTGTATCGAACCACATGATTGGACCAAGAACAT
AACAGACAAAATTGATCAGATTATTCATGATTTTG
TTGATAAAACCCTTCCGGACCAGGGGGACAATGAC
AATTGGTGGACAGGATGGAGACAATGGATACCGGC
AGGTATTGGAGTTACAGGTGTTATAATTGCAGTTA
TCGCTTTATTCTGTATATGCAAATTTGTCTTTTAG

A single A nucleotide (indicated below by a bold underlined letter) was added to the native 2014 EBOV GP sequence (SEQ ID 01) to create the full-length GP sequence (SEQ ID 02). The purpose of this addition was to eliminate expression of the secreted form of the Ebola glycoprotein (sGP) and to ensure that full-length GP will be expressed. (Volchkov et al., 1995), Virology 214,421-430). The GP sequence was translated in the EditSeq program (DNAStar) to verify that the sequence will express the full-length GP protein. SEQ ID: 03 is the product of the in silico translation.

SEQ ID 02:
Full-length 2014 EBOV GP
nucleotide sequence:
ATGGGTGTTACAGGAATATTGCAGTTACCTCGTGA
TCGATTCAAGAGGACATCATTCTTTCTTTGGGTAA
TTATCCTTTTCCAAAGAACATTTTCCATCCCGCTT
GGAGTTATCCACAATAGTACATTACAGGTTAGTGA
TGTCGACAAACTAGTTTGTCGTGACAAACTGTCAT
CCACAAATCAATTGAGATCAGTTGGACTGAATCTC
GAGGGGAATGGAGTGGCAACTGACGTGCCATCTGT
GACTAAAAGATGGGGCTTCAGGTCCGGTGTCCCAC
CAAAGGTGGTCAATTATGAAGCTGGTGAATGGGCT
GAAAACTGCTACAATCTTGAAATCAAAAAACCTGA
CGGGAGTGAGTGTCTACCAGCAGCGCCAGACGGGA
TTCGGGGCTTCCCCCGGTGCCGGTATGTGCACAAA
GTATCAGGAACGGGACCATGTGCCGGAGACTTTGC
CTTCCACAAAGAGGGTGCTTTCTTCCTGTATGATC
GACTTGCTTCCACAGTTATCTACCGAGGAACGACT
TTCGCTGAAGGTGTCGTTGCATTTCTGATACTGCC
CCAAGCTAAGAAGGACTTCTTCAGCTCACACCCCT
TGAGAGAGCCGGTCAATGCAACGGAGGACCCGTCG
AGTGGCTATTATTCTACCACAATTAGATATCAGGC
TACCGGTTTTGGAACTAATGAGACAGAGTACTTGT
TCGAGGTTGACAATTTGACCTACGTCCAACTTGAA
TCAAGATTCACACCACAGTTTCTGCTCCAGCTGAA
TGAGACAATATATGCAAGTGGGAAGAGGAGCAACA
CCACGGGAAAACTAATTTGGAAGGTCAACCCCGAA
ATTGATACAACAATCGGGGAGTGGGCCTTCTGGGA
AACTAAAAAAAACCTCACTAGAAAAATTCGCAGTG
AAGAGTTGTCTTTCACAGCTGTATCAAACGGACCC
AAAAACATCAGTGGTCAGAGTCCGGCGCGAACTTC
TTCCGACCCAGAGACCAACACAACAAATGAAGACC
ACAAAATCATGGCTTCAGAAAATTCCTCTGCAATG
GTTCAAGTGCACAGTCAAGGAAGGAAAGCTGCAGT
GTCGCATCTGACAACCCTTGCCACAATCTCCACGA
GTCCTCAACCTCCCACAACCAAAACAGGTCCGGAC
AACAGCACCCATAATACACCCGTGTATAAACTTGA
CATCTCTGAGGCAACTCAAGTTGGACAACATCACC
GTAGAGCAGACAACGACAGCACAGCCTCCGACACT
CCCCCCGCCACGACCGCAGCCGGACCCTTAAAAGC
AGAGAACACCAACACGAGTAAGAGCGCTGACTCCC
TGGACCTCGCCACCACGACAAGCCCCCAAAACTAC
AGCGAGACTGCTGGCAACAACAACACTCATCACCA
AGATACCGGAGAAGAGAGTGCCAGCAGCGGGAAGC
TAGGCTTAATTACCAATACTATTGCTGGAGTAGCA
GGACTGATCACAGGCGGGAGAAGGACTCGAAGAGA
AGTAATTGTCAATGCTCAACCCAAATGCAACCCCA
ATTTACATTACTGGACTACTCAGGATGAAGGTGCT
GCAATCGGATTGGCCTGGATACCATATTTCGGGCC
AGCAGCCGAAGGAATTTACACAGAGGGGCTAATGC
ACAACCAAGATGGTTTAATCTGTGGGTTGAGGCAG
CTGGCCAACGAAACGACTCAAGCTCTCCAACTGTT
CCTGAGAGCCACAACTGAGCTGCGAACCTTTTCAA
TCCTCAACCGTAAGGCAATTGACTTCCTGCTGCAG
CGATGGGGTGGCACATGCCACATTTTGGGACCGGA
CTGCTGTATCGAACCACATGATTGGACCAAGAACA
TAACAGACAAAATTGATCAGATTATTCATGATTTT
GTTGATAAAACCCTTCCGGACCAGGGGGACAATGA
CAATTGGTGGACAGGATGGAGACAATGGATACCGG
CAGGTATTGGAGTTACAGGTGTTATAATTGCAGTT
ATCGCTTTATTCTGTATATGCAAATTTGTCTTTTA
G
SEQ ID 03:
Full-length 2014 EBOV GP
protein sequence, generated
in EditSeq software from
SEQ ID 02:
MGVTGILQLPRDRFKRTSFFLWVIILFQRTFSIPL
GVIHNSTLQVSDVDKLVCRDKLSSTNQLRSVGLNL
EGNGVATDVPSVTKRWGFRSGVPPKVVNYEAGEWA
ENCYNLEIKKPDGSECLPAAPDGIRGFPRCRYVHK
VSGTGPCAGDFAFHKEGAFFLYDRLASTVIYRGTT
FAEGVVAFLILPQAKKDFFSSHPLREPVNATEDPS
SGYYSTTIRYQATGFGTNETEYLFEVDNLTYVQLE
SRFTPQFLLQLNETIYASGKRSNTTGKLIWKVNPE
IDTTIGEWAFWETKKNLTRKIRSEELSFTAVSNGP
KNISGQSPARTSSDPETNTTNEDHKIMASENSSAM
VQVHSQGRKAAVSHLTTLATISTSPQPPTTKTGPD
NSTHNTPVYKLD1SEATQVGQHHRRADNDSTASDT
PPATTAAGPLKAENTNTSKSADSLDLATTTSPQNY
SETAGNNNTHHQDTGEESASSGKLGLITNTIAGVA
GLITGGRRTRREVIVNAQPKCNPNLHYWTTQDEGA
AIGLAWIPYFGPAAEGIYTEGLMFINQDGLICGLR
QLANETTQALQLFLRATTELRTFSILNRKAIDFLL
QRWGGTCHILGPDCCIEPHDWTKNITDKIDQIIHD
FVDKTLPDQGDNDNWWTGWRQWIPAGIGVTGVIIA
VIALFCICKFVF

The full-length 2014 EBOV GP sequence (SEQ ID 02) was optimized for vaccinia virus expression using the online Gene Optimizer algorithm to generate SEQ ID 04.

SEQ ID 04:
Codon-optimized full-length
2014 EBOV GP sequence:
ATGGGAGTAACTGGAATTCTACAACTACCAAGAGA
TAGATTCAAAAGAACATCTTTTTTTCTATGGGTTA
TAATTCTATTTCAAAGAACATTTTCTATTCCATTG
GGAGTAATTCATAATTCTACATTGCAAGTATCTGA
TGTAGATAAACTAGTATGTAGAGATAAATTGTCTA
GTACAAATCAACTAAGATCTGTAGGATTGAATCTA
GAAGGAAATGGTGTAGCGACAGATGTTCCATCTGT
AACAAAAAGATGGGGTTTTAGATCTGGTGTACCAC
CAAAAGTAGTAAATTATGAAGCGGGAGAATGGGCG
GAAAATTGTTATAATCTAGAAATTAAAAAACCAGA
TGGATCTGAATGTCTACCAGCGGCGCCAGATGGAA
TTAGAGGATTTCCAAGATGTAGATATGTTCATAAA
GTATCTGGAACAGGACCATGTGCGGGAGATTTTGC
GTTTCATAAAGAAGGAGCATTTTTTCTATATGATA
GACTAGCGTCTACAGTAATATATAGAGGAACAACA
TTTGCGGAAGGTGTAGTAGCTTTTCTAATTCTACC
ACAAGCGAAAAAAGATTTTTTTAGTTCTCATCCAC
TAAGAGAACCAGTAAATGCGACAGAAGATCCTTCT
TCTGGATATTATTCTACTACAATTAGATATCAAGC
GACAGGATTTGGAACAAATGAAACAGAATATCTAT
TTGAAGTTGATAATCTAACATATGTACAACTAGAA
AGTAGATTCACACCACAATTTCTATTGCAATTGAA
TGAAACAATATATGCGTCTGGAAAAAGATCTAATA
CAACTGGAAAACTAATTTGGAAAGTAAATCCAGAA
ATTGATACAACAATTGGAGAATGGGCTTTTTGGGA
AACAAAAAAAAATTTGACAAGAAAAATTAGATCTG
AAGAATTGTCTTTTACAGCGGTATCTAATGGACCA
AAAAATATTTCTGGACAATCTCCAGCGAGAACTTC
TTCTGATCCAGAAACAAATACTACAAATGAAGATC
ACAAAATTATGGCGTCTGAAAATTCTTCTGCTATG
GTACAAGTACATTCTCAAGGAAGAAAAGCGGCGGT
ATCTCATCTAACAACACTAGCGACTATTTCTACAT
CTCCACAACCACCAACAACAAAAACTGGACCAGAT
AATAGTACACATAATACTCCAGTTTATAAACTAGA
TATTTCTGAAGCGACACAAGTTGGACAACATCATA
GAAGAGCGGATAATGATTCTACAGCGTCTGATACA
CCACCAGCTACAACAGCTGCTGGACCATTGAAAGC
GGAAAATACAAATACTTCTAAATCTGCGGATTCTC
TAGATTTGGCGACAACAACTTCTCCTCAAAATTAT
TCTGAAACAGCGGGAAATAATAATACTCATCATCA
AGATACTGGAGAAGAATCTGCGTCTAGTGGAAAAT
TGGGACTAATTACAAATACAATTGCGGGTGTAGCG
GGATTGATTACTGGTGGAAGAAGAACTAGAAGAGA
AGTAATAGTTAATGCGCAACCTAAATGTAATCCAA
ATCTACATTATTGGACAACTCAAGATGAAGGTGCT
GCGATTGGACTAGCTTGGATTCCATATTTTGGACC
TGCGGCGGAAGGAATATATACTGAAGGACTAATGC
ATAATCAAGATGGACTAATTTGTGGACTAAGACAA
CTAGCGAATGAAACTACACAAGCGCTACAACTATT
TTTGAGAGCGACAACAGAACTAAGAACTTTTAGTA
TTCTAAATAGAAAAGCGATTGATTTTTTGCTACAA
AGATGGGGAGGAACATGTCATATTCTAGGACCAGA
TTGTTGTATTGAACCACATGATTGGACAAAAAATA
TTACAGACAAAATTGATCAAATTATTCATGATTTT
GTTGATAAAACACTACCAGATCAAGGAGATAATGA
TAATTGGTGGACAGGATGGAGACAATGGATTCCAG
CGGGAATTGGAGTAACAGGTGTAATTATTGCGGTT
ATTGCGCTATTTTGTATATGTAAATTTGTTTTTTA
A

The codon-optimized full-length 2014 EBOV GP sequence (SEQ ID 04) was searched for homopolymer stretches consisting of ≥5 G bases or ≥C bases. None were found.

The codon-optimized full-length 2014 EBOV GP sequence (SEQ ID 04) was searched for homopolymer stretches consisting of ≥5 T bases or ≥A bases. Fifteen such stretches were found and were eliminated through silent mutations as listed in Table 3, to generate SEQ ID 05.

TABLE 3
Elimination of homopolymer stretches
in optimized 2014 EBOV GP sequence

| | | Changes | Mutation position | Codon change |
|---|---|---|---|---|
| | | (Silent | (base number) | (silent |
| No. | Homopolymer | mutation) | in sequence | mutation) |
| 1 | 7T | T to C | 57 | TTT to TTC |
| 2 | 5A | A to G | 252 | AAA to AAG |
| 3 | 6A | A to G | 342 | AAA to AAG |
| 4 | 6T | T to C | 477 | TTT to TTC |
| 5 | 6A | A to G | 570 | AAA to AAG |
| 6 | 7T | T to C | 579 | TTT to TTC |
| 7 | 6A | A to G | 795 | AAA to AAG |
| 8 | 5T | T to C | 870 | TTT to TTC |
| 9 | 9A | A to G | 882 | AAA to AAG |
| | | | 885 | |
| 10 | 5A | A to G | 900 | AAA to AAG |
| 11 | 6A | A to G | 948 | AAA to AAG |
| 12 | 5A | A to G | 1143 | AAA to AAG |
| 13 | 5T | T to C | 1716 | TTT to TTC |
| 14 | 6T | T to C | 1776 | TTT to TTC |
| 15 | 6A | A to G | 1851 | AAA to AAG |

*Shown as lower case in SEQ ID NO: 5

SEQ ID 05:
Homopolymer-free, codon-optimized,
full-length 2014 EBOV GP sequence:
ATGGGAGTAACTGGAATTCTACAACTACCAAGAGA
TAGATTCAAAAGAACATCTTTcTTTCTATGGGTTA
TAATTCTATTTCAAAGAACATTTTCTATTCCATTG
GGAGTAATTCATAATTCTACATTGCAAGTATCTGA
TGTAGATAAACTAGTATGTAGAGATAAATTGTCTA
GTACAAATCAACTAAGATCTGTAGGATTGAATCTA
GAAGGAAATGGTGTAGCGACAGATGTTCCATCTGT
AACAAAgAGATGGGGTTTTAGATCTGGTGTACCAC
CAAAAGTAGTAAATTATGAAGCGGGAGAATGGGCG
GAAAATTGTTATAATCTAGAAATTAAgAAACCAGA
TGGATCTGAATGTCTACCAGCGGCGCCAGATGGAA
TTAGAGGATTTCCAAGATGTAGATATGTTCATAAA
GTATCTGGAACAGGACCATGTGCGGGAGATTTTGC
GTTTCATAAAGAAGGAGCATTcTTTCTATATGATA
GACTAGCGTCTACAGTAATATATAGAGGAACAACA
TTTGCGGAAGGTGTAGTAGCTTTTCTAATTCTACC
ACAAGCGAAgAAAGATTTcTTTAGTTCTCATCCAC
TAAGAGAACCAGTAAATGCGACAGAAGATCCTTCT
TCTGGATATTATTCTACTACAATTAGATATCAAGC
GACAGGATTTGGAACAAATGAAACAGAATATCTAT
TTGAAGTTGATAATCTAACATATGTACAACTAGAA
AGTAGATTCACACCACAATTTCTATTGCAATTGAA
TGAAACAATATATGCGTCTGGAAAgAGATCTAATA
CAACTGGAAAACTAATTTGGAAAGTAAATCCAGAA
ATTGATACAACAATTGGAGAATGGGCTTTcTGGGA
AACAAAgAAgAATTTGACAAGAAAgATTAGATCTG
AAGAATTGTCTTTTACAGCGGTATCTAATGGACCA
AAgAATATTTCTGGACAATCTCCAGCGAGAACTTC
TTCTGATCCAGAAACAAATACTACAAATGAAGATC
ACAAAATTATGGCGTCTGAAAATTCTTCTGCTATG
GTACAAGTACATTCTCAAGGAAGAAAAGCGGCGGT
ATCTCATCTAACAACACTAGCGACTATTTCTACAT
CTCCACAACCACCAACAACAAAgACTGGACCAGAT
AATAGTACACATAATACTCCAGTTTATAAACTAGA
TATTTCTGAAGCGACACAAGTTGGACAACATCATA
GAAGAGCGGATAATGATTCTACAGCGTCTGATACA
CCACCAGCTACAACAGCTGCTGGACCATTGAAAGC
GGAAAATACAAATACTTCTAAATCTGCGGATTCTC
TAGATTTGGCGACAACAACTTCTCCTCAAAATTAT
TCTGAAACAGCGGGAAATAATAATACTCATCATCA
AGATACTGGAGAAGAATCTGCGTCTAGTGGAAAAT
TGGGACTAATTACAAATACAATTGCGGGTGTAGCG
GGATTGATTACTGGTGGAAGAAGAACTAGAAGAGA
AGTAATAGTTAATGCGCAACCTAAATGTAATCCAA
ATCTACATTATTGGACAACTCAAGATGAAGGTGCT
GCGATTGGACTAGCTTGGATTCCATATTTTGGACC
TGCGGCGGAAGGAATATATACTGAAGGACTAATGC
ATAATCAAGATGGACTAATTTGTGGACTAAGACAA
CTAGCGAATGAAACTACACAAGCGCTACAACTATT
cTTGAGAGCGACAACAGAACTAAGAACTTTTAGTA
TTCTAAATAGAAAAGCGATTGATTTcTTGCTACAA
AGATGGGGAGGAACATGTCATATTCTAGGACCAGA
TTGTTGTATTGAACCACATGATTGGACAAAgAATA
TTACAGACAAAATTGATCAAATTATTCATGATTTT
GTTGATAAAACACTACCAGATCAAGGAGATAATGA
TAATTGGTGGACAGGATGGAGACAATGGATTCCAG
CGGGAATTGGAGTAACAGGTGTAATTATTGCGGTT
ATTGCGCTATTTTGTATATGTAAATTTGTTTTTTA
A

The homopolymer-free, codon-optimized, full-length 2014 EBOV GP sequence (SEQ ID 05) was searched for vaccinia transcription terminator motifs. None were found.

A second stop codon and a vaccinia transcription terminator sequence were added at the end of the homopolymer-free, codon-optimized, full-length 2014 EBOV GP sequence (SEQ ID 05) to generate SEQ ID 06.

SEQ ID 06:
Homopolymer-free, codon-optimized,
full-length 2014 EBOV GP sequence
with stop codon and transcription
terminator added:
ATGGGAGTAACTGGAATTCTACAACTACCAAGAGA
TAGATTCAAAAGAACATCTTTcTTTCTATGGGTTA
TAATTCTATTTCAAAGAACATTTTCTATTCCATTG
GGAGTAATTCATAATTCTACATTGCAAGTATCTGA
TGTAGATAAACTAGTATGTAGAGATAAATTGTCTA
GTACAAATCAACTAAGATCTGTAGGATTGAATCTA
GAAGGAAATGGTGTAGCGACAGATGTTCCATCTGT
AACAAAgAGATGGGGTTTTAGATCTGGTGTACCAC
CAAAAGTAGTAAATTATGAAGCGGGAGAATGGGCG
GAAAATTGTTATAATCTAGAAATTAAgAAACCAGA
TGGATCTGAATGTCTACCAGCGGCGCCAGATGGAA
TTAGAGGATTTCCAAGATGTAGATATGTTCATAAA
GTATCTGGAACAGGACCATGTGCGGGAGATTTTGC
GTTTCATAAAGAAGGAGCATTcTTTCTATATGATA
GACTAGCGTCTACAGTAATATATAGAGGAACAACA
TTTGCGGAAGGTGTAGTAGCTTTTCTAATTCTACC
ACAAGCGAAgAAAGATTTcTTTAGTTCTCATCCAC
TAAGAGAACCAGTAAATGCGACAGAAGATCCTTCT
TCTGGATATTATTCTACTACAATTAGATATCAAGC
GACAGGATTTGGAACAAATGAAACAGAATATCTAT
TTGAAGTTGATAATCTAACATATGTACAACTAGAA
AGTAGATTCACACCACAATTTCTATTGCAATTGAA
TGAAACAATATATGCGTCTGGAAAgAGATCTAATA
CAACTGGAAAACTAATTTGGAAAGTAAATCCAGAA
ATTGATACAACAATTGGAGAATGGGCTTTcTGGGA
AACAAAgAAgAATTTGACAAGAAAgATTAGATCTG
AAGAATTGTCTTTTACAGCGGTATCTAATGGACCA
AAgAATATTTCTGGACAATCTCCAGCGAGAACTTC
TTCTGATCCAGAAACAAATACTACAAATGAAGATC
ACAAAATTATGGCGTCTGAAAATTCTTCTGCTATG
GTACAAGTACATTCTCAAGGAAGAAAAGCGGCGGT
ATCTCATCTAACAACACTAGCGACTATTTCTACAT
CTCCACAACCACCAACAACAAAgACTGGACCAGAT
AATAGTACACATAATACTCCAGTTTATAAACTAGA
TATTTCTGAAGCGACACAAGTTGGACAACATCATA
GAAGAGCGGATAATGATTCTACAGCGTCTGATACA
CCACCAGCTACAACAGCTGCTGGACCATTGAAAGC
GGAAAATACAAATACTTCTAAATCTGCGGATTCTC
TAGATTTGGCGACAACAACTTCTCCTCAAAATTAT
TCTGAAACAGCGGGAAATAATAATACTCATCATCA
AGATACTGGAGAAGAATCTGCGTCTAGTGGAAAAT
TGGGACTAATTACAAATACAATTGCGGGTGTAGCG
GGATTGATTACTGGTGGAAGAAGAACTAGAAGAGA
AGTAATAGTTAATGCGCAACCTAAATGTAATCCAA
ATCTACATTATTGGACAACTCAAGATGAAGGTGCT
GCGATTGGACTAGCTTGGATTCCATATTTTGGACC
TGCGGCGGAAGGAATATATACTGAAGGACTAATGC
ATAATCAAGATGGACTAATTTGTGGACTAAGACAA
CTAGCGAATGAAACTACACAAGCGCTACAACTATT
cTTGAGAGCGACAACAGAACTAAGAACTTTTAGTA
TTCTAAATAGAAAAGCGATTGATTTcTTGCTACAA
AGATGGGGAGGAACATGTCATATTCTAGGACCAGA
TTGTTGTATTGAACCACATGATTGGACAAAgAATA
TTACAGACAAAATTGATCAAATTATTCATGATTTT
GTTGATAAAACACTACCAGATCAAGGAGATAATGA
TAATTGGTGGACAGGATGGAGACAATGGATTCCAG
CGGGAATTGGAGTAACAGGTGTAATTATTGCGGTT
ATTGCGCTATTTTGTATATGTAAATTTGTTTTTTA
ATAATTTTTAT

The native nucleotide sequence for 2014 EBOV VP40 was obtained from GenBank (accession number KM233103.1)

SEQ ID 07:
Native nucleotide sequence
for 2014 EBOV VP40, from
GenBank:
ATGAGGCGGGTTATATTGCCTACTGCTCCTCCTGA
ATATATGGAGGCCATATACCCTGCCAGGTCAAATT
CAACAATTGCTAGGGGTGGCAACAGCAATACAGGC
TTCCTGACACCGGAGTCAGTCAATGGAGACACTCC
ATCGAATCCACTCAGGCCAATTGCTGATGACACCA
TCGACCATGCCAGCCACACACCAGGCAGTGTGTCA
TCAGCATTCATCCTCGAAGCTATGGTGAATGTCAT
ATCGGGCCCCAAAGTGCTAATGAAGCAAATTCCAA
TTTGGCTTCCTCTAGGTGTCGCTGATCAAAAGACC
TACAGCTTTGACTCAACTACGGCCGCCATCATGCT
TGCTTCATATACTATCACCCATTTCGGCAAGGCAA
CCAATCCGCTTGTCAGAGTCAATCGGCTGGGTCCT
GGAATCCCGGATCACCCCCTCAGGCTCCTGCGAAT
TGGAAACCAGGCTTTCCTCCAGGAGTTCGTTCTTC
CACCAGTCCAACTACCCCAGTATTTCACCTTTGAT
TTGACAGCACTCAAACTGATCACTCAACCACTGCC
TGCTGCAACATGGACCGATGACACTCCAACTGGAT
CAAATGGAGCGTTGCGTCCAGGAATTTCATTTCAT
CCAAAACTTCGCCCCATTCTTTTACCCAACAAAAG
TGGGAAGAAGGGGAACAGTGCCGATCTAACATCTC
CGGAGAAAATCCAAGCAATAATGACTTCACTCCAG
GACTTTAAGATCGTTCCAATTGATCCAACCAAAAA
TATCATGGGTATCGAAGTGCCAGAAACTCTGGTCC
ACAAGCTGACCGGTAAGAAGGTGACTTCCAAAAAT
GGACAACCAATCATCCCTGTTCTTTTGCCAAAGTA
CATTGGGTTGGACCCGGTGGCTCCAGGAGACCTCA
CCATGGTAATCACACAGGATTGTGACACGTGTCAT
TCTCCTGCAAGTCTTCCAGCTGTGGTTGAGAAGTA
A

The native nucleotide sequence for 2014 EBOV VP40 (SEQ ID 07) was optimized for vaccinia virus expression using the online Gene Optimizer algorithm.

The codon-optimized 2014 EBOV VP40 sequence was searched for homopolymer stretches consisting of ≥5 G bases or ≥C bases. None were found.

The codon-optimized 2014 EBOV VP40 sequence was searched for homopolymer stretches consisting of ≥5 T bases or ≥5 A bases. Five such stretches were found and were eliminated through silent mutations as listed in Table 4, to generate SEQ ID 08.

TABLE 4
Elimination of homopolymer stretches
in optimized 2014 EBOV VP40 sequence

| | | Changes | Mutation position | Codon change |
|---|---|---|---|---|
| | | (Silent | (base number) | (silent |
| No. | Homopolymer | mutation) | in sequence | mutation) |
| 1 | 6A | A to G | 312 | AAA to AAG |
| 2 | 7A | A to G | 672 | AAA to AAG |
| 3 | 6A | A to G | 708 | AAA to AAG |
| 4 | 6A | A to G | 768 | AAA to AAG |
| 5 | 7A | A to G | 822 | AAA to AAG |

*Shown as lower case in SEQ ID NO: 8 and SEQ ID NO: 9

SEQ ID 08:
Homopolymer-free, codon-optimized
2014 EBOV VP40 sequence:
ATGAGAAGAGTAATTCTACCAACAGCGCCACCAGA
ATATATGGAAGCGATATATCCAGCGAGATCTAATT
CTACAATTGCGAGAGGTGGAAATTCTAATACTGGA
TTTCTAACACCAGAATCTGTAAATGGAGATACACC
ATCTAATCCACTAAGACCAATTGCGGATGATACAA
TAGATCATGCGAGTCATACTCCAGGATCTGTATCT
TCTGCTTTTATTCTAGAAGCTATGGTTAATGTAAT
TTCTGGACCAAAAGTACTAATGAAACAAATTCCAA
TTTGGCTACCATTGGGAGTAGCGGATCAAAAgACA
TATTCTTTTGATTCTACTACAGCGGCGATTATGCT
AGCGTCTTATACAATTACACATTTTGGAAAAGCGA
CAAATCCACTAGTTAGAGTAAATAGACTAGGACCT
GGAATACCAGATCATCCATTGAGACTACTAAGAAT
TGGAAATCAAGCTTTTCTACAAGAATTTGTTCTAC
CACCAGTACAACTACCACAATACTTTACATTTGAT
CTAACAGCGCTAAAACTAATTACACAACCATTGCC
AGCGGCGACATGGACAGATGATACACCAACAGGAT
CTAATGGTGCTCTAAGACCTGGTATTTCTTTTCAT
CCAAAACTAAGACCTATTCTATTGCCAAATAAATC
TGGAAAgAAAGGAAATTCTGCGGATCTAACATCTC
CAGAAAAgATTCAAGCGATTATGACATCTCTACAA
GACTTCAAAATTGTACCAATTGATCCAACAAAgAA
TATTATGGGAATTGAAGTACCAGAAACACTAGTTC
ATAAACTAACTGGAAAgAAAGTAACATCTAAAAAT
GGACAACCTATTATTCCAGTATTGCTACCTAAATA
TATTGGACTAGATCCAGTAGCGCCTGGAGATCTAA
CAATGGTTATTACACAAGATTGTGATACTTGTCAT
TCTCCAGCGAGTTTGCCTGCGGTAGTAGAAAAATA
A

The homopolymer-free, codon-optimized, full-length 2014 EBOV GP sequence (SEQ ID 08) was searched for vaccinia transcription terminator motifs. None were found.

A second stop codon and a vaccinia transcription terminator sequence were added at the end of the homopolymer-free, codon-optimized 2014 EBOV VP40 sequence (SEQ ID 08) to generate SEQ ID 09.

SEQ ID 09:
Homopolymer-free, codon-optimized
2014 EBOV VP40 sequence with stop
codon and transcription terminator
added:
ATGAGAAGAGTAATTCTACCAACAGCGCCACCAGA
ATATATGGAAGCGATATATCCAGCGAGATCTAATT
CTACAATTGCGAGAGGTGGAAATTCTAATACTGGA
TTTCTAACACCAGAATCTGTAAATGGAGATACACC
ATCTAATCCACTAAGACCAATTGCGGATGATACAA
TAGATCATGCGAGTCATACTCCAGGATCTGTATCT
TCTGCTTTTATTCTAGAAGCTATGGTTAATGTAAT
TTCTGGACCAAAAGTACTAATGAAACAAATTCCAA
TTTGGCTACCATTGGGAGTAGCGGATCAAAAgACA
TATTCTTTTGATTCTACTACAGCGGCGATTATGCT
AGCGTCTTATACAATTACACATTTTGGAAAAGCGA
CAAATCCACTAGTTAGAGTAAATAGACTAGGACCT
GGAATACCAGATCATCCATTGAGACTACTAAGAAT
TGGAAATCAAGCTTTTCTACAAGAATTTGTTCTAC
CACCAGTACAACTACCACAATACTTTACATTTGAT
CTAACAGCGCTAAAACTAATTACACAACCATTGCC
AGCGGCGACATGGACAGATGATACACCAACAGGAT
CTAATGGTGCTCTAAGACCTGGTATTTCTTTTCAT
CCAAAACTAAGACCTATTCTATTGCCAAATAAATC
TGGAAAgAAAGGAAATTCTGCGGATCTAACATCTC
CAGAAAAgATTCAAGCGATTATGACATCTCTACAA
GACTTCAAAATTGTACCAATTGATCCAACAAAgAA
TATTATGGGAATTGAAGTACCAGAAACACTAGTTC
ATAAACTAACTGGAAAgAAAGTAACATCTAAAAAT
GGACAACCTATTATTCCAGTATTGCTACCTAAATA
TATTGGACTAGATCCAGTAGCGCCTGGAGATCTAA
CAATGGTTATTACACAAGATTGTGATACTTGTCAT
TCTCCAGCGAGTTTGCCTGCGGTAGTAGAAAAATA
ATAATTTTTAT

### Example 3: Additional Antigen Sequences for Filovirus MVA Vaccine

In another exemplary embodiment, sequences from Zaire Ebola (ZEBOV) and Sudan Ebola Virus (SUDV) are prepared in shuttle plasmids and optimized. Viral sequences are then inserted into MVA vector vaccines described herein. These sequences are modified from native sequences using the methods described herein.

TABLE 5
Zaire Ebola VP40 mutation table

| Changes | Mutation position |
|---|---|
| (Silent mutation) | on VP40 |
| A to G | 312 |
| A to G | 672 |
| A to G | 708 |
| A to G | 768 |
| A to G | 822 |

*Shown as lower case in SEQ ID NO: 10

SEQ ID 10:
pGEO-ZEBOV2014 VP40 sequence optimized
for insertion into MVA vector:
(FIG. 3)
GAATTCGGAGTATACGAACCGGGAAAGAGAAGATG
GTTAAAAATAAAGCGAGACTATTTGAACGAGGGTT
CCATGGCAGATTCTGCCGATTTAGTAGTACTAGGT
GCTTACTATGGTAAAGGAGCAAAGGGTGGTATCAT
GGCAGTCTTTCTAATGGGTTGTTACGACGATGAAT
CCGGTAAATGGAAGACGGTTACCAAGTGTTCAGGA
CACGATGATAATACGTTAAGGGAGTTGCAAGACCA
ATTAAAGATGATTAAAATTAACAAGGATCCCAAAA
AAATTCCAGAGTGGTTAGTAGTTAATAAAATCTAT
ATTCCCGATTTTGTAGTAGAGGATCCAAAACAATC
TCAGATATGGGAAATTTCAGGAGCAGAGTTTACAT
CTTCCAAGTCCCATACCGCAAATGGAATATCCATT
AGATTTCCTAGATTTACTAGGATAAGAGAGGATAA
AACGTGGAAAGAATCTACTCATCTAAACGATTTAG
TAAACTTGACTAAATCTTAATTTTTATGGCGCGCC
TTTCATTTTGTTTTTTTCTATGCTATAAATGGTGA
GCAAGGGCGAGGAGCTGTTCACCGGGGTGGTGCCC
ATCCTGGTCGAGCTGGACGGCGACGTAAACGGCCA
CAAGTTCAGCGTGTCCGGCGAGGGCGAGGGCGATG
CCACCTACGGCAAGCTGACCCTGAAGTTCATCTGC
ACCACCGGCAAGCTGCCCGTGCCCTGGCCCACCCT
CGTGACCACCCTGACCTACGGCGTGCAGTGCTTCA
GCCGCTACCCCGACCACATGAAGCAGCACGACTTC
TTCAAGTCCGCCATGCCCGAAGGCTACGTCCAGGA
GCGCACCATCTTCTTCAAGGACGACGGCAACTACA
AGACCCGCGCCGAGGTGAAGTTCGAGGGCGACACC
CTGGTGAACCGCATCGAGCTGAAGGGCATCGACTT
CAAGGAGGACGGCAACATCCTGGGGCACAAGCTGG
AGTACAACTACAACAGCCACAACGTCTATATCATG
GCCGACAAGCAGAAGAACGGCATCAAGGTGAACTT
CAAGATCCGCCACAACATCGAGGACGGCAGCGTGC
AGCTCGCCGACCACTACCAGCAGAACACCCCCATC
GGCGACGGCCCCGTGCTGCTGCCCGACAACCACTA
CCTGAGCACCCAGTCCGCCCTGAGCAAAGACCCCA
ACGAGAAGCGCGATCACATGGTCCTGCTGGAGTTC
GTGACCGCCGCCGGGATCACTCTCGGCATGCACGA
GCTGTACAAGTAAGAGCTCCCCGATTTTGTAGTAG
AGGATCCAAAACAATCTCAGATATGGGAAATTTCA
GGAGCAGAGTTTACATCTTCCAAGTCCCATACCGC
AAATGGAATATCCATTAGATTTCCTAGATTTACTA
GGATAAGAGAGGATAAAACGTGGAAAGAATCTACT
CATCTAAACGATTTAGTAAACTTGACTAAATCTTA
ATTTTTATCTCGAGGCCGCTGGTACCCAACCTAAA
AATTGAAAATAAATACAAAGGTTCTTGAGGGTTGT
GTTAAATTGAAAGCGAGAAATAATCATAAATAAGC
CCgggATGAGAAGAGTAATTCTACCAACAGCGCCA
CCAGAATATATGGAAGCGATATATCCAGCGAGATC
TAATTCTACAATTGCGAGAGGTGGAAATTCTAATA
CTGGATTTCTAACACCAGAATCTGTAAATGGAGAT
ACACCATCTAATCCACTAAGACCAATTGCGGATGA
TACAATAGATCATGCGAGTCATACTCCAGGATCTG
TATCTTCTGCTTTTATTCTAGAAGCTATGGTTAAT
GTAATTTCTGGACCAAAAGTACTAATGAAACAAAT
TCCAATTTGGCTACCATTGGGAGTAGCGGATCAAA
AgACATATTCTTTTGATTCTACTACAGCGGCGATT
ATGCTAGCGTCTTATACAATTACACATTTTGGAAA
AGCGACAAATCCACTAGTTAGAGTAAATAGACTAG
GACCTGGAATACCAGATCATCCATTGAGACTACTA
AGAATTGGAAATCAAGCTTTTCTACAAGAATTTGT
TCTACCACCAGTACAACTACCACAATACTTTACAT
TTGATCTAACAGCGCTAAAACTAATTACACAACCA
TTGCCAGCGGCGACATGGACAGATGATACACCAAC
AGGATCTAATGGTGCTCTAAGACCTGGTATTTCTT
TTCATCCAAAACTAAGACCTATTCTATTGCCAAAT
AAATCTGGAAAgAAAGGAAATTCTGCGGATCTAAC
ATCTCCAGAAAAgATTCAAGCGATTATGACATCTC
TACAAGACTTCAAAATTGTACCAATTGATCCAACA
AAgAATATTATGGGAATTGAAGTACCAGAAACACT
AGTTCATAAACTAACTGGAAAgAAAGTAACATCTA
AAAATGGACAACCTATTATTCCAGTATTGCTACCT
AAATATATTGGACTAGATCCAGTAGCGCCTGGAGA
TCTAACAATGGTTATTACACAAGATTGTGATACTT
GTCATTCTCCAGCGAGTTTGCCTGCGGTAGTAGAA
AAATAATAATTTTTATgTCGACCTGCAGCTAATGT
ATTAGTTAAATATTAAAACTTACCACGTAAAACTT
AAAATTTAAAATGATATTTCATTGACAGATAGATC
ACACATTATGAACTTTCAAGGACTTGTGTTAACTG
ACAATTGCAAAAATCAATGGGTCGTTGGACCATTA
ATAGGAAAAGGTGGATTTGGTAGTATTTATACTAC
TAATGACAATAATTATGTAGTAAAAATAGAGCCCA
AAGCTAACGGATCATTATTTACCGAACAGGCATTT
TATACTAGAGTACTTAAACCATCCGTTATCGAAGA
ATGGAAAAAATCTCACAATATAAAGCACGTAGGTC
TTATCACGTGCAAGGCATTTGGTCTATACAAATCC
ATTAATGTGGAATATCGATTCTTGGTAATTAATAG
ATTAGGTGCAGATCTAGATGCGGTGATCAGAGCCA
ATAATAATAGATTACCAAAAAGGTCGGTGATGTTG
ATCGGAATCGAAATCTTAAATACCATACAATTTAT
GCACGAGCAAGGATATTCTCACGGAGATATTAAAG
CGAGTAATATAGTCTTGGATCAAATAGATAAGAAT
AAATTATATCTAGTGGATTACGGATTGGTTTCTAA
ATTCATGTCAAGCTTGTCTCCCTATAGTGAGTCGT
ATTAGAGCTTGGCGTAATCATGGTCATAGCTGTTT
CCTGTGTGAAATTGTTATCCGCTCACAATTCCACA
CAACATACGAGCCGGAAGCATAAAGTGTAAAGCCT
GGGGTGCCTAATGAGTGAGCTAACTCACATTAATT
GCGTTGCGCTCACTGCCCGCTTTCGAGTCGGGAAA
CCTGTCGTGCCAGCTGCATTAATGAATCGGCCAAC
GCGCGGGGAGAGGCGGTTTGCGTATTGGGCGCTCT
TCCGCTTCCTCGCTCACTGACTCGCTGCGCTCGGT
CGTTCGGCTGCGGCGAGCGGTATCAGCTCACTCAA
AGGCGGTAATACGGTTATCCACAGAATCAGGGGAT
AACGCAGGAAAGAACATGTGAGCAAAAGGCCAGCA
AAAGGCCAGGAACCGTAAAAAGGCCGCGTTGCTGG
CGTTTTTCGATAGGCTCCGCCCCCCTGACGAGCAT
CACAAAAATCGACGCTCAAGTCAGAGGTGGCGAAA
CCCGACAGGACTATAAAGATACCAGGCGTTTCCCC
CTGGAAGCTCCCTCGTGCGCTCTCCTGTTCCGACC
CTGCCGCTTACCGGATACCTGTCCGCCTTTCTCCC
TTCGGGAAGCGTGGCGCTTTCTCATAGCTCACGCT
GTAGGTATCTCAGTTCGGTGTAGGTCGTTCGCTCC
AAGCTGGGCTGTGTGCACGAACCCCCCGTTCAGCC
CGACCGCTGCGCCTTATCCGGTAACTATCGTCTTG
AGTCCAACCCGGTAAGACACGACTTATCGCCACTG
GCAGCAGCCACTGGTAACAGGATTAGCAGAGCGAG
GTATGTAGGCGGTGCTACAGAGTTCTTGAAGTGGT
GGCCTAACTACGGCTACACTAGAAGGACAGTATTT
GGTATCTGCGCTCTGCTGAAGCCAGTTACCTTCGG
AAAAAGAGTTGGTAGCTCTTGATCCGGCAAACAAA
CCACCGCTGGTAGCGGTGGTTTTTTTGTTTGCAAG
CAGCAGATTACGCGCAGAAAAAAAGGATCTCAAGA
AGATCCTTTGATCTTTTCTACGGGGTCTGACGCTC
AGTGGAACGAAAACTCACGTTAAGGGATTTTGGTC
ATGAGATTATCAAAAAGGATCTTCACCTAGATCCT
TTTAAATTAAAAATGAAGTTTTAAATCAATCTAAA
GTATATATGAGTAAACTTGGTCTGACAGTTACCAA
TGCTTAATCAGTGAGGCACCTATCTCAGCGATCTG
TCTATTTCGTTCATCCATAGTTGCCTGACTCCCCG
TCGTGTAGATAACTACGATACGGGAGGGCTTACCA
TCTGGCCCCAGTGCTGCAATGATACCGCGAGACCC
ACGCTCACCGGCTCCAGATTTATCAGCAATAAACC
AGCCAGCCGGAAGGGCCGAGCGCAGAAGTGGTCCT
GCAACTTTATCCGCCTCCATCCAGTCTATTAATTG
TTGCCGGGAAGCTAGAGTAAGTAGTTCGCCAGTTA
ATAGTTTGCGCAACGTTGTTGGCATTGCTACAGGC
ATCGTGGTGTCACGCTCGTCGTTTGGTATGGCTTC
ATTCAGCTCCGGTTCCCAACGATCAAGGCGAGTTA
CATGATCCCCCATGTTGTGCAAAAAAGCGGTTAGC
TCCTTCGGTCCTCCGATCGTTGTCAGAAGTAAGTT
GGCCGCAGTGTTATCACTCATGGTTATGGCAGCAC
TGCATAATTCTCTTACTGTCATGCCATCCGTAAGA
TGCTTTTCTGTGACTGGTGAGTACTCAACCAAGTC
ATTCTGAGAATAGTGTATGCGGCGACCGAGTTGCT
CTTGCCCGGCGTCAATACGGGATAATACCGCGCCA
CATAGCAGAACTTTAAAAGTGCTCATCATTGGAAA
ACGTTCTTCGGGGCGAAAACTCTCAAGGATCTTAC
CGCTGTTGAGATCCAGTTCGATGTAACCCACTCGT
GCACCCAACTGATCTTCAGCATCTTTTACTTTCAC
CAGCGTTTCTGGGTGAGCAAAAACAGGAAGGCAAA
ATGCCGCAAAAAAGGGAATAAGGGCGACACGGAAA
TGTTGAATACTCATACTCTTCCTTTTTCAATATTA
TTGAAGCATTTATCAGGGTTATTGTCTCATGAGCG
GATACATATTTGAATGTATTTAGAAAAATAAACAA
ATAGGGGTTCCGCGCACATTTCCCCGAAAAGTGCC
ACCTGACGTCTAAGAAACCATTATTATCATGACAT
TAACCTATAAAAATAGGCGTATCACGAGGCCCTTT
CGTCTCGCGCGTTTCGGTGATGACGGTGAAAACCT
CTGACACATGCAGCTCCCGGAGACGGTCACAGCTT
GTCTGTAAGCGGATGCCGGGAGCAGACAAGCCCGT
CAGGGCGCGTCAGCGGGTGTTGGCGGGTGTCGGGG
CTGGCTTAACTATGCGGCATCAGAGCAGATTGTAC
TGAGAGTGCACCATATGCGGTGTGAAATACCGCAC
AGATGCGTAAGGAGAAAATACCGCATCAGGCGCCA
TTCGCCATTCAGGCTGCGCAACTGTTGGGAAGGGC
GATCGGTGCGGGCCTCTTCGCTATTACGCCAGCTG
GCGAAAGGGGGATGTGCTGCAAGGCGATTAAGTTG
GGTAACGCCAGGGTTTTCCCAGTCACGACGTTGTA
AAACGACGGCCAGTGAATTGGATTTAGGTGACACT
ATA

TABLE 6
Zaire Ebola Glycoprotein mutation table

| Changes | Mutation position |
|---|---|
| (Silent mutation) | on GP |
| T to C | 57 |
| A to G | 252 |
| A to G | 342 |
| T to C | 477 |
| A to G | 570 |
| T to C | 579 |
| A to G | 795 |
| T to C | 870 |
| A to G | 882 |
| | 885 |
| A to G | 900 |
| A to G | 948 |
| A to G | 1143 |
| T to C | 1716 |
| T to C | 1776 |
| A to G | 1851 |

*Shown as lower case in SEQ ID NO: 11

SEQ ID 11:
pGEO-ZEBOV2014 GP sequence optimized
for insertion into MVA vector:
(FIG. 2)
GAATTCCCTGGGACATACGTATATTTCTATGATCT
GTCTTATATGAAGTCTATACAGCGAATAGATTCAG
AATTTCTACATAATTATATATTGTACGCTAATAAG
TTTAATCTAACACTCCCCGAAGATTTGTTTATAAT
CCCTACAAATTTGGATATTCTATGGCGTACAAAGG
AATATATAGACTCGTTCGATATTAGTACAGAAACA
TGGAATAAATTATTATCCAATTATTATATGAAGAT
GATAGAGTATGCTAAACTTTATGTACTAAGTCCTA
TTCTCGCTGAGGAGTTGGATAATTTTGAGAGGACG
GGAGAATTAACTAGTATTGTACAAGAAGCCATTTT
ATCTCTAAATTTACGAATTAAGATTTTAAATTTTA
AACATAAAGATGATGATACGTATATACACTTTTGT
AAAATATTATTCGGTGTCTATAACGGAACAAACGC
TACTATATATTATCATAGACCTCTAACGGGATATA
TGAATATGATTTCAGATACTATATTTGTTCCTGTA
GATAATAACTAAGGCGCGCCTTTCATTTTGTTTTT
TTCTATGCTATAAATGGTGAGCAAGGGCGAGGAGC
TGTTCACCGGGGTGGTGCCCATCCTGGTCGAGCTG
GACGGCGACGTAAACGGCCACAAGTTCAGCGTGTC
CGGCGAGGGCGAGGGCGATGCCACCTACGGCAAGC
TGACCCTGAAGTTCATCTGCACCACCGGCAAGCTG
CCCGTGCCCTGGCCCACCCTCGTGACCACCCTGAC
CTACGGCGTGCAGTGCTTCAGCCGCTACCCCGACC
ACATGAAGCAGCACGACTTCTTCAAGTCCGCCATG
CCCGAAGGCTACGTCCAGGAGCGCACCATCTTCTT
CAAGGACGACGGCAACTACAAGACCCGCGCCGAGG
TGAAGTTCGAGGGCGACACCCTGGTGAACCGCATC
GAGCTGAAGGGCATCGACTTCAAGGAGGACGGCAA
CATCCTGGGGCACAAGCTGGAGTACAACTACAACA
GCCACAACGTCTATATCATGGCCGACAAGCAGAAG
AACGGCATCAAGGTGAACTTCAAGATCCGCCACAA
CATCGAGGACGGCAGCGTGCAGCTCGCCGACCACT
ACCAGCAGAACACCCCCATCGGCGACGGCCCCGTG
CTGCTGCCCGACAACCACTACCTGAGCACCCAGTC
CGCCCTGAGCAAAGACCCCAACGAGAAGCGCGATC
ACATGGTCCTGCTGGAGTTCGTGACCGCCGCCGGG
ATCACTCTCGGCATGCACGAGCTGTACAAGTAAGA
GCTCGAGGACGGGAGAATTAACTAGTATTGTACAA
GAAGCCATTTTATCTCTAAATTTACGAATTAAGAT
TTTAAATTTTAAACATAAAGATGATGATACGTATA
TACACTTTTGTAAAATATTATTCGGTGTCTATAAC
GGAACAAACGCTACTATATATTATCATAGACCTCT
AACGGGATATATGAATATGATTTCAGATACTATAT
TTGTTCCTGTAGATAATAACTAACTCGAGGCCGCT
GGTACCCAACCTAAAAATTGAAAATAAATACAAAG
GTTCTTGAGGGTTGTGTTAAATTGAAAGCGAGAAA
TAATCATAAATAAGCCCgggATGGGAGTAACTGGA
ATTCTACAACTACCAAGAGATAGATTCAAAAGAAC
ATCTTTcTTTCTATGGGTTATAATTCTATTTCAAA
GAACATTTTCTATTCCATTGGGAGTAATTCATAAT
TCTACATTGCAAGTATCTGATGTAGATAAACTAGT
ATGTAGAGATAAATTGTCTAGTACAAATCAACTAA
GATCTGTAGGATTGAATCTAGAAGGAAATGGTGTA
GCGACAGATGTTCCATCTGTAACAAAgAGATGGGG
TTTTAGATCTGGTGTACCACCAAAAGTAGTAAATT
ATGAAGCGGGAGAATGGGCGGAAAATTGTTATAAT
CTAGAAATTAAgAAACCAGATGGATCTGAATGTCT
ACCAGCGGCGCCAGATGGAATTAGAGGATTTCCAA
GATGTAGATATGTTCATAAAGTATCTGGAACAGGA
CCATGTGCGGGAGATTTTGCGTTTCATAAAGAAGG
AGCATTcTTTCTATATGATAGACTAGCGTCTACAG
TAATATATAGAGGAACAACATTTGCGGAAGGTGTA
GTAGCTTTTCTAATTCTACCACAAGCGAAgAAAGA
TTTcTTTAGTTCTCATCCACTAAGAGAACCAGTAA
ATGCGACAGAAGATCCTTCTTCTGGATATTATTCT
ACTACAATTAGATATCAAGCGACAGGATTTGGAAC
AAATGAAACAGAATATCTATTTGAAGTTGATAATC
TAACATATGTACAACTAGAAAGTAGATTCACACCA
CAATTTCTATTGCAATTGAATGAAACAATATATGC
GTCTGGAAAgAGATCTAATACAACTGGAAAACTAA
TTTGGAAAGTAAATCCAGAAATTGATACAACAATT
GGAGAATGGGCTTTcTGGGAAACAAAgAAgAATTT
GACAAGAAAgATTAGATCTGAAGAATTGTCTTTTA
CAGCGGTATCTAATGGACCAAAgAATATTTCTGGA
CAATCTCCAGCGAGAACTTCTTCTGATCCAGAAAC
AAATACTACAAATGAAGATCACAAAATTATGGCGT
CTGAAAATTCTTCTGCTATGGTACAAGTACATTCT
CAAGGAAGAAAAGCGGCGGTATCTCATCTAACAAC
ACTAGCGACTATTTCTACATCTCCACAACCACCAA
CAACAAAgACTGGACCAGATAATAGTACACATAAT
ACTCCAGTTTATAAACTAGATATTTCTGAAGCGAC
ACAAGTTGGACAACATCATAGAAGAGCGGATAATG
ATTCTACAGCGTCTGATACACCACCAGCTACAACA
GCTGCTGGACCATTGAAAGCGGAAAATACAAATAC
TTCTAAATCTGCGGATTCTCTAGATTTGGCGACAA
CAACTTCTCCTCAAAATTATTCTGAAACAGCGGGA
AATAATAATACTCATCATCAAGATACTGGAGAAGA
ATCTGCGTCTAGTGGAAAATTGGGACTAATTACAA
ATACAATTGCGGGTGTAGCGGGATTGATTACTGGT
GGAAGAAGAACTAGAAGAGAAGTAATAGTTAATGC
GCAACCTAAATGTAATCCAAATCTACATTATTGGA
CAACTCAAGATGAAGGTGCTGCGATTGGACTAGCT
TGGATTCCATATTTTGGACCTGCGGCGGAAGGAAT
ATATACTGAAGGACTAATGCATAATCAAGATGGAC
TAATTTGTGGACTAAGACAACTAGCGAATGAAACT
ACACAAGCGCTACAACTATTcTTGAGAGCGACAAC
AGAACTAAGAACTTTTAGTATTCTAAATAGAAAAG
CGATTGATTTcTTGCTACAAAGATGGGGAGGAACA
TGTCATATTCTAGGACCAGATTGTTGTATTGAACC
ACATGATTGGACAAAgAATATTACAGACAAAATTG
ATCAAATTATTCATGATTTTGTTGATAAAACACTA
CCAGATCAAGGAGATAATGATAATTGGTGGACAGG
ATGGAGACAATGGATTCCAGCGGGAATTGGAGTAA
CAGGTGTAATTATTGCGGTTATTGCGCTATTTTGT
ATATGTAAATTTGTTTTTTAATAATTTTTATgTCG
ACCTGCAGTCAAACTCTAATGACCACATCTTTTTT
TAGAGATGAAAAATTTTCCACATCTCCTTTTGTAG
ACACGACTAAACATTTTGCAGAAAAAAGTTTATTA
GTGTTTAGATAATCGTATACTTCATCAGTGTAGAT
AGTAAATGTGAACAGATAAAAGGTATTCTTGCTCA
ATAGATTGGTAAATTCCATAGAATATATTAATCCT
TTCTTCTTGAGATCCCACATCATTTCAACCAGAGA
CGTTTTATCCAATGATTTACCTCGTACTATACCAC
ATACAAAACTAGATTTTGCAGTGACGTCGTATCTG
GTATTCCTACCAAACAAAATTTTACTTTTAGTTCT
TTTAGAAAATTCTAAGGTAGAATCTCTATTTGCCA
ATATGTCATCTATGGAATTACCACTAGCAAAAAAT
GATAGAAATATATATTGATACATCGCAGCTGGTTT
TGATCTACTATACTTTAAAAACGAATCAGATTCCA
TAATTGCCTGTATATCATCAGCTGAAAAACTATGT
TTTACACGTATTCCTTCGGCATTTCTTTTTAATGA
TATATCTTGTTTAGACAATGATAAAGTTATCATGT
CCATGAGAGACGCGTCTCCGTATCGTATAAATATT
TCATTAGATGTTAGACGCTTCATTAGGGGTATACT
TCTATAAGGTTTCTTAATCAGTCCATCATTGGTTG
CGTCAAGAACAAGCTTGTCTCCCTATAGTGAGTCG
TATTAGAGCTTGGCGTAATCATGGTCATAGCTGTT
TCCTGTGTGAAATTGTTATCCGCTCACAATTCCAC
ACAACATACGAGCCGGAAGCATAAAGTGTAAAGCC
TGGGGTGCCTAATGAGTGAGCTAACTCACATTAAT
TGCGTTGCGCTCACTGCCCGCTTTCGAGTCGGGAA
ACCTGTCGTGCCAGCTGCATTAATGAATCGGCCAA
CGCGCGGGGAGAGGCGGTTTGCGTATTGGGCGCTC
TTCCGCTTCCTCGCTCACTGACTCGCTGCGCTCGG
TCGTTCGGCTGCGGCGAGCGGTATCAGCTCACTCA
AAGGCGGTAATACGGTTATCCACAGAATCAGGGGA
TAACGCAGGAAAGAACATGTGAGCAAAAGGCCAGC
AAAAGGCCAGGAACCGTAAAAAGGCCGCGTTGCTG
GCGTTTTTCGATAGGCTCCGCCCCCCTGACGAGCA
TCACAAAAATCGACGCTCAAGTCAGAGGTGGCGAA
ACCCGACAGGACTATAAAGATACCAGGCGTTTCCC
CCTGGAAGCTCCCTCGTGCGCTCTCCTGTTCCGAC
CCTGCCGCTTACCGGATACCTGTCCGCCTTTCTCC
CTTCGGGAAGCGTGGCGCTTTCTCATAGCTCACGC
TGTAGGTATCTCAGTTCGGTGTAGGTCGTTCGCTC
CAAGCTGGGCTGTGTGCACGAACCCCCCGTTCAGC
CCGACCGCTGCGCCTTATCCGGTAACTATCGTCTT
GAGTCCAACCCGGTAAGACACGACTTATCGCCACT
GGCAGCAGCCACTGGTAACAGGATTAGCAGAGCGA
GGTATGTAGGCGGTGCTACAGAGTTCTTGAAGTGG
TGGCCTAACTACGGCTACACTAGAAGGACAGTATT
TGGTATCTGCGCTCTGCTGAAGCCAGTTACCTTCG
GAAAAAGAGTTGGTAGCTCTTGATCCGGCAAACAA
ACCACCGCTGGTAGCGGTGGTTTTTTTGTTTGCAA
GCAGCAGATTACGCGCAGAAAAAAAGGATCTCAAG
AAGATCCTTTGATCTTTTCTACGGGGTCTGACGCT
CAGTGGAACGAAAACTCACGTTAAGGGATTTTGGT
CATGAGATTATCAAAAAGGATCTTCACCTAGATCC
TTTTAAATTAAAAATGAAGTTTTAAATCAATCTAA
AGTATATATGAGTAAACTTGGTCTGACAGTTACCA
ATGCTTAATCAGTGAGGCACCTATCTCAGCGATCT
GTCTATTTCGTTCATCCATAGTTGCCTGACTCCCC
GTCGTGTAGATAACTACGATACGGGAGGGCTTACC
ATCTGGCCCCAGTGCTGCAATGATACCGCGAGACC
CACGCTCACCGGCTCCAGATTTATCAGCAATAAAC
CAGCCAGCCGGAAGGGCCGAGCGCAGAAGTGGTCC
TGCAACTTTATCCGCCTCCATCCAGTCTATTAATT
GTTGCCGGGAAGCTAGAGTAAGTAGTTCGCCAGTT
AATAGTTTGCGCAACGTTGTTGGCATTGCTACAGG
CATCGTGGTGTCACGCTCGTCGTTTGGTATGGCTT
CATTCAGCTCCGGTTCCCAACGATCAAGGCGAGTT
ACATGATCCCCCATGTTGTGCAAAAAAGCGGTTAG
CTCCTTCGGTCCTCCGATCGTTGTCAGAAGTAAGT
TGGCCGCAGTGTTATCACTCATGGTTATGGCAGCA
CTGCATAATTCTCTTACTGTCATGCCATCCGTAAG
ATGCTTTTCTGTGACTGGTGAGTACTCAACCAAGT
CATTCTGAGAATAGTGTATGCGGCGACCGAGTTGC
TCTTGCCCGGCGTCAATACGGGATAATACCGCGCC
ACATAGCAGAACTTTAAAAGTGCTCATCATTGGAA
AACGTTCTTCGGGGCGAAAACTCTCAAGGATCTTA
CCGCTGTTGAGATCCAGTTCGATGTAACCCACTCG
TGCACCCAACTGATCTTCAGCATCTTTTACTTTCA
CCAGCGTTTCTGGGTGAGCAAAAACAGGAAGGCAA
AATGCCGCAAAAAAGGGAATAAGGGCGACACGGAA
ATGTTGAATACTCATACTCTTCCTTTTTCAATATT
ATTGAAGCATTTATCAGGGTTATTGTCTCATGAGC
GGATACATATTTGAATGTATTTAGAAAAATAAACA
AATAGGGGTTCCGCGCACATTTCCCCGAAAAGTGC
CACCTGACGTCTAAGAAACCATTATTATCATGACA
TTAACCTATAAAAATAGGCGTATCACGAGGCCCTT
TCGTCTCGCGCGTTTCGGTGATGACGGTGAAAACC
TCTGACACATGCAGCTCCCGGAGACGGTCACAGCT
TGTCTGTAAGCGGATGCCGGGAGCAGACAAGCCCG
TCAGGGCGCGTCAGCGGGTGTTGGCGGGTGTCGGG
GCTGGCTTAACTATGCGGCATCAGAGCAGATTGTA
CTGAGAGTGCACCATATGCGGTGTGAAATACCGCA
CAGATGCGTAAGGAGAAAATACCGCATCAGGCGCC
ATTCGCCATTCAGGCTGCGCAACTGTTGGGAAGGG
CGATCGGTGCGGGCCTCTTCGCTATTACGCCAGCT
GGCGAAAGGGGGATGTGCTGCAAGGCGATTAAGTT
GGGTAACGCCAGGGTTTTCCCAGTCACGACGTTGT
AAAACGACGGCCAGTGAATTGGATTTAGGTGACAC
TATA

TABLE 7
Sudan Ebola VP40 mutation table

| Changes | Mutation position |
|---|---|
| (Silent mutation) | on VP40 |
| A to G | 111 |
| A to G | 312 |
| A to G | 663 |
| A to G | 675 |
| A to G | 822 |
| A to G | 837 |
| A to G | 978 |

*Shown as lower case in SEQ ID NO: 12

SEQ ID 12:
pGEO-SUDV2014 VP40 sequence optimized for insertion into MVA vector: (FIG. 7)
GAATTCGGAGTATACGAACCGGGAAAGAGAAGATGGTTAAAAATAAAGCGAGACTATTTGAACGAGGGTTCCATGGC
AGATTCTGCCGATTTAGTAGTACTAGGTGCTTACTATGGTAAAGGAGCAAAGGGTGGTATCATGGCAGTCTTTCTAA
TGGGTTGTTACGACGATGAATCCGGTAAATGGAAGACGGTTACCAAGTGTTCAGGACACGATGATAATACGTTAAGG
GAGTTGCAAGACCAATTAAAGATGATTAAAATTAACAAGGATCCCAAAAAAATTCCAGAGTGGTTAGTAGTTAATAA
AATCTATATTCCCGATTTTGTAGTAGAGGATCCAAAACAATCTCAGATATGGGAAATTTCAGGAGCAGAGTTTACAT
CTTCCAAGTCCCATACCGCAAATGGAATATCCATTAGATTTCCTAGATTTACTAGGATAAGAGAGGATAAAACGTGG
AAAGAATCTACTCATCTAAACGATTTAGTAAACTTGACTAAATCTTAATTTTTATGGCGCGCCTTTCATTTTGTTTT
TTTCTATGCTATAAATGGTGAGCAAGGGCGAGGAGCTGTTCACCGGGGTGGTGCCCATCCTGGTCGAGCTGGACGGC
GACGTAAACGGCCACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTACGGCGTGCAGTGCTTCA
GCCGCTACCCCGACCACATGAAGCAGCACGACTTCTTCAAGTCCGCCATGCCCGAAGGCTACGTCCAGGAGCGCACC
ATCTTCTTCAAGGACGACGGCAACTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAACATCGAGGACGGC
AGCGTGCAGCTCGCCGACCACTACCAGCAGAACACCCCCATCGGCGACGGCCCCGTGCTGCTGCCCGACAACCACTA
CCTGAGCACCCAGTCCGCCCTGAGCAAAGACCCCAACGAGAAGCGCGATCACATGGTCCTGCTGGAGTTCGTGACCG
CCGCCGGGATCACTCTCGGCATGCACGAGCTGTACAAGTAAGAGCTCCCCGATTTTGTAGTAGAGGATCCAAAACAA
TCTCAGATATGGGAAATTTCAGGAGCAGAGTTTACATCTTCCAAGTCCCATACCGCAAATGGAATATCCATTAGATT
TCCTAGATTTACTAGGATAAGAGAGGATAAAACGTGGAAAGAATCTACTCATCTAAACGATTTAGTAAACTTGACTA
AATCTTAATTTTTATCTCGAGGCCGCTGGTACCCAACCTAAAAATTGAAAATAAATACAAAGGTTCTTGAGGGTTGT
GTTAAATTGAAAGCGAGAAATAATCATAAATAAGCCCgggATGAAAAGAGTAACAGTACCAACAGCGCCACCAGCGT
ATGCGGATATAGGATATCCAATGTCTATGCTACCTATTAAATCTTCTAGAGCGGTATCTGGAATTCAACAAAAgCAA
GAAGTACTACCTGGAATGGATACACCATCTAATTCTATGAGACCAGTAGCGGATGATAATATTGATCATACTTCTCA
TACTCCAAATGGTGTAGCGTCTGCTTTTATTCTAGAAGCGACAGTAAATGTAATTTCTGGACCAAAAGTACTAATGA
AACAAATTCCAATTTGGCTACCACTAGGAATTGCGGATCAAAAgACATATTCTTTTGATTCTACAACAGCGGCGATT
ATGCTAGCGTCTTATACAATTACACATTTTGGAAAAGCGAATAATCCACTAGTTAGAGTAAATAGACTAGGACAAGG
AATACCAGATCATCCACTAAGACTACTAAGAATGGGAAATCAAGCTTTTCTACAAGAATTTGTTCTACCACCAGTAC
AACTACCACAATACTTTACATTTGATCTAACAGCGCTAAAACTAGTAACACAACCACTACCAGCGGCGACATGGACA
GATGAAACTCCATCTAATCTAAGTGGTGCTCTAAGACCAGGACTATCTTTTCATCCAAAACTAAGACCTGTACTACT
ACCAGGAAAgACTGGAAAgAAAGGACATGTATCTGATTTGACAGCGCCAGACAAAATTCAAACAATAGTAAATCTAA
TGCAAGACTTCAAAATTGTACCAATTGATCCAGCGAAATCTATTATTGGAATTGAAGTACCAGAACTACTAGTTCAT
AAATTGACTGGAAAgAAAATGTCTCAAAAgAATGGACAACCTATTATTCCAGTACTATTGCCTAAATATATTGGTCT
AGATCCTATTTCTCCTGGAGATCTAACAATGGTTATTACACCAGATTATGATGATTGTCATTCTCCAGCGTCTTGTT
CTTATCTATCTGAAAAgTAAtaagTCGACCTGCAGCTAATGTATTAGTTAAATATTAAAACTTACCACGTAAAACTT
AAAATTTAAAATGATATTTCATTGACAGATAGATCACACATTATGAACTTTCAAGGACTTGTGTTAACTGACAATTG
CAAAAATCAATGGGTCGTTGGACCATTAATAGGAAAAGGTGGATTTGGTAGTATTTATACTACTAATGACAATAATT
ATGTAGTAAAAATAGAGCCCAAAGCTAACGGATCATTATTTACCGAACAGGCATTTTATACTAGAGTACTTAAACCA
TCCGTTATCGAAGAATGGAAAAAATCTCACAATATAAAGCACGTAGGTCTTATCACGTGCAAGGCATTTGGTCTATA
CAAATCCATTAATGTGGAATATCGATTCTTGGTAATTAATAGATTAGGTGCAGATCTAGATGCGGTGATCAGAGCCA
ATAATAATAGATTACCAAAAAGGTCGGTGATGTTGATCGGAATCGAAATCTTAAATACCATACAATTTATGCACGAG
CAAGGATATTCTCACGGAGATATTAAAGCGAGTAATATAGTCTTGGATCAAATAGATAAGAATAAATTATATCTAGT
GGATTACGGATTGGTTTCTAAATTCATGTCAAGCTTGTCTCCCTATAGTGAGTCGTATTAGAGCTTGGCGTAATCAT
GGTCATAGCTGTTTCCTGTGTGAAATTGTTATCCGCTCACAATTCCACACAACATACGAGCCGGAAGCATAAAGTGT
AAAGCCTGGGGTGCCTAATGAGTGAGCTAACTCACATTAATTGCGTTGCGCTCACTGCCCGCTTTCGAGTCGGGAAA
CCTGTCGTGCCAGCTGCATTAATGAATCGGCCAACGCGCGGGGAGAGGCGGTTTGCGTATTGGGCGCTCTTCCGCTT
CCTCGCTCACTGACTCGCTGCGCTCGGTCGTTCGGCTGCGGCGAGCGGTATCAGCTCACTCAAAGGCGGTAATACGG
TTATCCACAGAATCAGGGGATAACGCAGGAAAGAACATGTGAGCAAAAGGCCAGCAAAAGGCCAGGAACCGTAAAAA
GGCCGCGTTGCTGGCGTTTTTCGATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCTCGTGCGCTCTCCTGTTCCGACC
CTGCCGCTTACCGGATACCTGTCCGCCTTTCTCCCTTCGGGAAGCGTGGCGCTTTCTCATAGCTCACGCTGTAGGTA
TCTCAGTTCGGTGTAGGTCGTTCGCTCCAAGCTGGGCTGTGTGCACGAACCCCCCGTTCAGCCCGACCGCTGCGCCT
TATCCGGTAACTATCGTCTTGAGTCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCTAACTACGGCTACACTAGAAGGAC
AGTATTTGGTATCTGCGCTCTGCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAAAAAGGATCTCAAGAAGATCCT
TTGATCTTTTCTACGGGGTCTGACGCTCAGTGGAACGAAAACTCACGTTAAGGGATTTTGGTCATGAGATTATCAAA
AAGGATCTTCACCTAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATCCATAGTTGCCTGA
CTCCCCGTCGTGTAGATAACTACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGCGCAGAAGTGGTCCTGCAACTT
TATCCGCCTCCATCCAGTCTATTAATTGTTGCCGGGAAGCTAGAGTAAGTAGTTCGCCAGTTAATAGTTTGCGCAAC
GTTGTTGGCATTGCTACAGGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTCCTCCGATCGTTGTCAGAA
GTAAGTTGGCCGCAGTGTTATCACTCATGGTTATGGCAGCACTGCATAATTCTCTTACTGTCATGCCATCCGTAAGA
TGCTTTTCTGTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTTCTTCGGGGC
GAAAACTCTCAAGGATCTTACCGCTGTTGAGATCCAGTTCGATGTAACCCACTCGTGCACCCAACTGATCTTCAGCA
TCTTTTACTTTCACCAGCGTTTCTGGGTGAGCAAAAACAGGAAGGCAAAATGCCGCAAAAAAGGGAATAAGGGCGAC
ACGGAAATGTTGAATACTCATACTCTTCCTTTTTCAATATTATTGAAGCATTTATCAGGGTTATTGTCTCATGAGCG
GATACATATTTGAATGTATTTAGAAAAATAAACAAATAGGGGTTCCGCGCACATTTCCCCGAAAAGTGCCACCTGAC
GTCTAAGAAACCATTATTATCATGACATTAACCTATAAAAATAGGCGTATCACGAGGCCCTTTCGTCTCGCGCGTTT
CGGTGATGACGGTGAAAACCTCTGACACATGCAGCTCCCGGAGACGGTCACAGCTTGTCTGTAAGCGGATGCCGGGA
GCAGACAAGCCCGTCAGGGCGCGTCAGCGGGTGTTGGCGGGTGTCGGGGCTGGCTTAACTATGCGGCATCAGAGCAG
ATTGTACTGAGAGTGCACCATATGCGGTGTGAAATACCGCACAGATGCGTAAGGAGAAAATACCGCATCAGGCGCCA
TTCGCCATTCAGGCTGCGCAACTGTTGGGAAGGGCGATCGGTGCGGGCCTCTTCGCTATTACGCCAGCTGGCGAAAG
GGGGATGTGCTGCAAGGCGATTAAGTTGGGTAACGCCAGGGTTTTCCCAGTCACGACGTTGTAAAACGACGGCCAGT
GAATTGGATTTAGGTGACACTATA

TABLE 8
Sudan Ebola Glycoprotein mutation table

| Changes | Mutation position |
|---|---|
| (Silent mutation) | on GP |
| T to C | 57 |
| A to G | 87 |
| T to C | 93 |
| A to G | 252 |
| A to G | 342 |
| T to C | 477 |
| A to G | 882, 885 |
| A to G | 1035 |
| A to G | 1407 |
| A to G | 1491 |
| T to C | 1776 |
| A to G | 1851 |

*Shown as lower case in SEQ ID NO: 13

SEQ ID 13:
pGEO-SUDV2014 GP sequence optimized for insertion into MVA vector: (FIG. 8)
GAATTCCCTGGGACATACGTATATTTCTATGATCTGTCTTATATGAAGTCTATACAGCGAATAGATTCAGAATTT
CTACATAATTATATATTGTACGCTAATAAGTTTAATCTAACACTCCCCGAAGATTTGTTTATAATCCCTACAAAT
TTGGATATTCTATGGCGTACAAAGGAATATATAGACTCGTTCGATATTAGTACAGAAACATGGAATAAATTATTA
TCCAATTATTATATGAAGATGATAGAGTATGCTAAACTTTATGTACTAAGTCCTATTCTCGCTGAGGAGTTGGAT
AATTTTGAGAGGACGGGAGAATTAACTAGTATTGTACAAGAAGCCATTTTATCTCTAAATTTACGAATTAAGATT
TTAAATTTTAAACATAAAGATGATGATACGTATATACACTTTTGTAAAATATTATTCGGTGTCTATAACGGAACA
AACGCTACTATATATTATCATAGACCTCTAACGGGATATATGAATATGATTTCAGATACTATATTTGTTCCTGTA
GATAATAACTAAGGCGCGCCTTTCATTTTGTTTTTTTCTATGCTATAAATGGTGAGCAAGGGCGAGGAGCTGTTC
ACCGGGGTGGTGCCCATCCTGGTCGAGCTGGACGGCGACGTAAACGGCCACAAGTTCAGCGTGTCCGGCGAGGGC
GAGGGCGATGCCACCTACGGCAAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACCACATGAAGCAGCACGACTTC
TTCAAGTCCGCCATGCCCGAAGGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAGGGCATCGACTTCAAGGAGGAC
GGCAACATCCTGGGGCACAAGCTGGAGTACAACTACAACAGCCACAACGTCTATATCATGGCCGACAAGCAGAAG
AACGGCATCAAGGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCACCCAGTCCGCCCTGAGC
AAAGACCCCAACGAGAAGCGCGATCACATGGTCCTGCTGGAGTTCGTGACCGCCGCCGGGATCACTCTCGGCATG
CACGAGCTGTACAAGTAAGAGCTCGAGGACGGGAGAATTAACTAGTATTGTACAAGAAGCCATTTTATCTCTAAA
TTTACGAATTAAGATTTTAAATTTTAAACATAAAGATGATGATACGTATATACACTTTTGTAAAATATTATTCGG
TGTCTATAACGGAACAAACGCTACTATATATTATCATAGACCTCTAACGGGATATATGAATATGATTTCAGATAC
TATATTTGTTCCTGTAGATAATAACTAACTCGAGGCCGCTGGTACCCAACCTAAAAATTGAAAATAAATACAAAG
GTTCTTGAGGGTTGTGTTAAATTGAAAGCGAGAAATAATCATAAATAAGCCCgggATGGGAGGACTATCTCTACT
ACAACTACCAAGAGATAAGTTTAGAAAATCTTCTTTcTTTGTTTGGGTTATAATTCTATTTCAAAAgGCGTTcTC
TATGCCATTGGGAGTAGTAACAAATTCTACACTAGAAGTAACAGAAATTGATCAACTAGTATGTAAAGATCATCT
AGCGTCTACAGATCAATTGAAATCTGTTGGATTGAATCTAGAAGGATCTGGTGTATCTACAGATATTCCATCTGC
GACAAAgAGATGGGGTTTTAGAAGTGGTGTACCACCAAAAGTAGTATCTTATGAAGCGGGAGAATGGGCGGAAAA
TTGTTATAATCTAGAAATTAAgAAACCAGATGGATCTGAATGTTTGCCACCACCACCAGATGGTGTTAGAGGATT
TCCAAGATGTAGATATGTTCATAAAGCGCAAGGAACAGGACCATGTCCTGGAGATTATGCGTTTCATAAAGATGG
TGCATTcTTTCTATATGATAGATTGGCGTCTACTGTAATATATAGAGGTGTAAATTTTGCGGAAGGTGTAATTGC
TTTTCTAATTCTAGCGAAACCTAAAGAAACATTTCTACAATCTCCACCAATTAGAGAAGCGGTTAATTATACAGA
AAATACTTCATCTTATTATGCGACATCTTATCTAGAATATGAAATTGAAAATTTTGGAGCGCAACATTCTACAAC
TTTGTTCAAAATTGATAATAATACTTTTGTTAGACTAGATAGACCACATACACCACAATTTTTGTTTCAATTGAA
TGATACAATTCATCTACATCAACAACTATCTAATACAACTGGAAGATTGATTTGGACACTAGATGCGAATATTAA
TGCGGATATTGGAGAATGGGCTTTcTGGGAAAATAAgAAgAATCTATCTGAACAACTAAGAGGAGAAGAATTGTC
TTTTGAAGCGCTATCTCTAAATGAAACTGAAGATGATGATGCGGCGTCTAGTAGAATTACAAAAGGAAGAATTTC
TGATAGAGCGACAAGACAATATTCTGATCTAGTACCAAAgAATCCACCTGGAATGGTTCCATTGCATATTCCAGA
AGGAGAAACAACACTACCATCTCAAAATTCTACTGAAGGAAGAAGAGTATCTGTAAATACTCAAGAAACAATTAC
AGAAACAGCGGCGACAATTATTGGAACAAATGGAAATCATATGCAAATTTCTACTATTGGAATTAGACCATCTTC
TTCTCAAATTCCATCTTCTAGTCCAACAACAGCGCCATCTCCAGAAGCGCAAACACCAACAACACATACAAGTGG
ACCATCTGTAATGGCGACAGAAGAACCTACAACACCACCAGGATCTTCTCCAGGTCCAACTACAGAAGCGCCAAC
TCTAACTACACCAGAAAATATTACAACAGCTGTAAAgACAGTACTACCACAAGAATCTACTTCTAATGGACTAAT
TACATCTACAGTAACTGGAATTCTAGGATCTCTAGGACTAAGAAAgAGATCTAGAAGACAAACAAATACAAAAGC
GACTGGAAAATGTAATCCAAATCTACATTATTGGACAGCGCAAGAACAACATAATGCGGCGGGAATTGCTTGGAT
TCCATATTTTGGACCAGGTGCTGAAGGAATATATACTGAAGGTCTAATGCATAATCAAAATGCGCTAGTATGTGG
ACTAAGACAACTAGCGAATGAAACAACTCAAGCGCTACAACTATTTCTAAGAGCGACTACAGAACTAAGAACATA
TACAATTCTAAATAGAAAAGCTATTGATTTcTTGTTGAGAAGATGGGGAGGAACATGTAGAATATTGGGACCAGA
TTGTTGTATTGAACCACATGATTGGACAAAgAATATTACTGACAAAATTAATCAAATTATTCATGACTTTATTGA
TAATCCACTACCAAATCAAGATAATGATGATAATTGGTGGACAGGATGGAGACAATGGATTCCAGCGGGAATAGG
AATTACTGGAATTATTATTGCGATTATAGCGCTACTATGTGTATGTAAACTACTATGTTAATAAgTCGACCTGCA
GTCAAACTCTAATGACCACATCTTTTTTTAGAGATGAAAAATTTTCCACATCTCCTTTTGTAGACACGACTAAAC
ATTTTGCAGAAAAAAGTTTATTAGTGTTTAGATAATCGTATACTTCATCAGTGTAGATAGTAAATGTGAACAGAT
AAAAGGTATTCTTGCTCAATAGATTGGTAAATTCCATAGAATATATTAATCCTTTCTTCTTGAGATCCCACATCA
TTTCAACCAGAGACGTTTTATCCAATGATTTACCTCGTACTATACCACATACAAAACTAGATTTTGCAGTGACGT
CGTATCTGGTATTCCTACCAAACAAAATTTTACTTTTAGTTCTTTTAGAAAATTCTAAGGTAGAATCTCTATTTG
CCAATATGTCATCTATGGAATTACCACTAGCAAAAAATGATAGAAATATATATTGATACATCGCAGCTGGTTTTG
ATCTACTATACTTTAAAAACGAATCAGATTCCATAATTGCCTGTATATCATCAGCTGAAAAACTATGTTTTACAC
GTATTCCTTCGGCATTTCTTTTTAATGATATATCTTGTTTAGACAATGATAAAGTTATCATGTCCATGAGAGACG
CGTCTCCGTATCGTATAAATATTTCATTAGATGTTAGACGCTTCATTAGGGGTATACTTCTATAAGGTTTCTTAA
TCAGTCCATCATTGGTTGCGTCAAGAACAAGCTTGTCTCCCTATAGTGAGTCGTATTAGAGCTTGGCGTAATCAT
GGTCATAGCTGTTTCCTGTGTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCACTGCCCGCTTTCGAGTCGG
GAAACCTGTCGTGCCAGCTGCATTAATGAATCGGCCAACGCGCGGGGAGAGGCGGTTTGCGTATTGGGCGCTCTT
CCGCTTCCTCGCTCACTGACTCGCTGCGCTCGGTCGTTCGGCTGCGGCGAGCGGTATCAGCTCACTCAAAGGCGG
TAATACGGTTATCCACAGAATCAGGGGATAACGCAGGAAAGAACATGTGAGCAAAAGGCCAGCAAAAGGCCAGGA
ACCGTAAAAAGGCCGCGTTGCTGGCGTTTTTCGATAGGCTCCGCCCCCCTGACGAGCATCACAAAAATCGACGCT
CAAGTCAGAGGTGGCGAAACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTGTGCACGAACCCCCCGTTC
AGCCCGACCGCTGCGCCTTATCCGGTAACTATCGTCTTGAGTCCAACCCGGTAAGACACGACTTATCGCCACTGG
CAGCAGCCACTGGTAACAGGATTAGCAGAGCGAGGTATGTAGGCGGTGCTACAGAGTTCTTGAAGTGGTGGCCTA
ACTACGGCTACACTAGAAGGACAGTATTTGGTATCTGCGCTCTGCTGAAGCCAGTTACCTTCGGAAAAAGAGTTG
GTAGCTCTTGATCCGGCAAACAAACCACCGCTGGTAGCGGTGGTTTTTTTGTTTGCAAGCAGCAGATTACGCGCA
GAAAAAAAGGATCTCAAGAAGATCCTTTGATCTTTTCTACGGGGTCTGACGCTCAGTGGAACGAAAACTCACGTT
AAGGGATTTTGGTCATGAGATTATCAAAAAGGATCTTCACCTAGATCCTTTTAAATTAAAAATGAAGTTTTAAAT
CAATCTAAAGTATATATGAGTAAACTTGGTCTGACAGTTACCAATGCTTAATCAGTGAGGCACCTATCTCAGCGA
TCTGTCTATTTCGTTCATCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATCAGCAATAAACCAGCCAG
CCGGAAGGGCCGAGCGCAGAAGTGGTCCTGCAACTTTATCCGCCTCCATCCAGTCTATTAATTGTTGCCGGGAAG
CTAGAGTAAGTAGTTCGCCAGTTAATAGTTTGCGCAACGTTGTTGGCATTGCTACAGGCATCGTGGTGTCACGCT
CGTCGTTTGGTATGGCTTCATTCAGCTCCGGTTCCCAACGATCAAGGCGAGTTACATGATCCCCCATGTTGTGCA
AAAAAGCGGTTAGCTCCTTCGGTCCTCCGATCGTTGTCAGAAGTAAGTTGGCCGCAGTGTTATCACTCATGGTTA
TGGCAGCACTGCATAATTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAACTCTCAAGGATCTTACCGCTGT
TGAGATCCAGTTCGATGTAACCCACTCGTGCACCCAACTGATCTTCAGCATCTTTTACTTTCACCAGCGTTTCTG
GGTGAGCAAAAACAGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATATTTGAATGTATTT
AGAAAAATAAACAAATAGGGGTTCCGCGCACATTTCCCCGAAAAGTGCCACCTGACGTCTAAGAAACCATTATTA
TCATGACATTAACCTATAAAAATAGGCGTATCACGAGGCCCTTTCGTCTCGCGCGTTTCGGTGATGACGGTGAAA
ACCTCTGACACATGCAGCTCCCGGAGACGGTCACAGCTTGTCTGTAAGCGGATGCCGGGAGCAGACAAGCCCGTC
AGGGCGCGTCAGCGGGTGTTGGCGGGTGTCGGGGCTGGCTTAACTATGCGGCATCAGAGCAGATTGTACTGAGAG
TGCACCATATGCGGTGTGAAATACCGCACAGATGCGTAAGGAGAAAATACCGCATCAGGCGCCATTCGCCATTCA
GGCTGCGCAACTGTTGGGAAGGGCGATCGGTGCGGGCCTCTTCGCTATTACGCCAGCTGGCGAAAGGGGGATGTG
CTGCAAGGCGATTAAGTTGGGTAACGCCAGGGTTTTCCCAGTCACGACGTTGTAAAACGACGGCCAGTGAATTGG
ATTTAGGTGACACTATA

### Example 4: MVA Vaccine Incorporating Marburg Virus Sequences

In an exemplary embodiment, sequences from Marburg virus (MARV) are prepared and optimized in shuttle plasmids and then the viral sequences are incorporated into an MVA vector. Such MVA vectors may be used individually as part of an administration protocol to elicit an immune response to Marburg virus or as part of a multivalent vaccine composition having one or more MVA vectors expressing EBOV and Marburg antigens to elicit an immune response.

TABLE 9
Marburg VP40 mutation table

| Changes | Mutation position |
|---|---|
| (Silent mutation) | on VP40 |
| A to G | 357 |
| T to C | 465 |
| A to G | 519 |
| A to G | 630 |
| A to G | 654 |
| A to G | 717 |
| A to G | 729 |
| A to G | 792 |

*Shown as lower case in SEQ ID NO: 14

Exemplary Marburg VP40 and GP sequences are provided below.

SEQ ID 14:
pGEO-MARV2014 VP40 sequence optimized for insertion into MVA vector: (FIG. 9)
GAATTCGGAGTATACGAACCGGGAAAGAGAAGATGGTTAAAAATAAAGCGAGACTATTTGAACGAGGGTTCCATGGC
AGATTCTGCCGATTTAGTAGTACTAGGTGCTTACTATGGTAAAGGAGCAAAGGGTGGTATCATGGCAGTCTTTCTAA
TGGGTTGTTACGACGATGAATCCGGTAAATGGAAGACGGTTACCAAGTGTTCAGGACACGATGATAATACGTTAAGG
GAGTTGCAAGACCAATTAAAGATGATTAAAATTAACAAGGATCCCAAAAAAATTCCAGAGTGGTTAGTAGTTAATAA
AATCTATATTCCCGATTTTGTAGTAGAGGATCCAAAACAATCTCAGATATGGGAAATTTCAGGAGCAGAGTTTACAT
CTTCCAAGTCCCATACCGCAAATGGAATATCCATTAGATTTCCTAGATTTACTAGGATAAGAGAGGATAAAACGTGG
AAAGAATCTACTCATCTAAACGATTTAGTAAACTTGACTAAATCTTAATTTTTATGGCGCGCCTTTCATTTTGTTTT
TTTCTATGCTATAAATGGTGAGCAAGGGCGAGGAGCTGTTCACCGGGGTGGTGCCCATCCTGGTCGAGCTGGACGGC
GACGTAAACGGCCACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTACGGCGTGCAGTGCTTCA
GCCGCTACCCCGACCACATGAAGCAGCACGACTTCTTCAAGTCCGCCATGCCCGAAGGCTACGTCCAGGAGCGCACC
ATCTTCTTCAAGGACGACGGCAACTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAACATCGAGGACGGC
AGCGTGCAGCTCGCCGACCACTACCAGCAGAACACCCCCATCGGCGACGGCCCCGTGCTGCTGCCCGACAACCACTA
CCTGAGCACCCAGTCCGCCCTGAGCAAAGACCCCAACGAGAAGCGCGATCACATGGTCCTGCTGGAGTTCGTGACCG
CCGCCGGGATCACTCTCGGCATGCACGAGCTGTACAAGTAAGAGCTCCCCGATTTTGTAGTAGAGGATCCAAAACAA
TCTCAGATATGGGAAATTTCAGGAGCAGAGTTTACATCTTCCAAGTCCCATACCGCAAATGGAATATCCATTAGATT
TCCTAGATTTACTAGGATAAGAGAGGATAAAACGTGGAAAGAATCTACTCATCTAAACGATTTAGTAAACTTGACTA
AATCTTAATTTTTATCTCGAGGCCGCTGGTACCCAACCTAAAAATTGAAAATAAATACAAAGGTTCTTGAGGGTTGT
GTTAAATTGAAAGCGAGAAATAATCATAAATAAGCCCgggATGGCGTCTAGTTCTAATTATAATACTTATATGCAAT
ATCTAAATCCACCACCATATGCGGATCATGGTGCTAATCAACTAATTCCAGCGGATCAACTATCTAATCAACATGGA
ATTACACCAAATTATGTTGGAGATCTAAATCTAGATGATCAGTTTAAAGGAAATGTTTGTCATGCGTTTACACTAGA
AGCGATTATTGATATTTCTGCGTATAATGAAAGAACAGTAAAAGGTGTACCAGCTTGGCTACCACTAGGAATTATGT
CTAATTTTGAATATCCACTAGCGCATACAGTAGCGGCGCTATTGACAGGATCTTATACAATTACACAGTTTACACAT
AATGGACAAAAgTTTGTTAGAGTAAATAGACTAGGAACTGGAATACCAGCGCATCCACTAAGAATGCTAAGAGAAGG
AAATCAAGCTTTTATTCAAAATATGGTTATTCCAAGAAATTTcTCTACAAATCAGTTTACTTATAATCTAACTAATC
TAGTACTATCTGTACAAAAgCTACCAGATGATGCTTGGAGACCATCTAAAGATAAACTAATTGGAAATACAATGCAT
CCAGCGATTTCTATTCATCCAAATCTACCACCAATAGTACTACCAACTGTAAAgAAACAAGCGTATAGACAACATAA
gAATCCAAATAATGGACCACTATTGGCGATTTCTGGAATTCTACATCAACTAAGAGTAGAAAAgGTACCAGAAAAgA
CATCTTTGTTTAGAATTTCTCTACCAGCGGATATGTTTTCTGTAAAAGAAGGAATGATGAAgAAAAGAGGAGAATCT
TCTCCAGTAGTATATTTTCAAGCGCCAGAAAATTTTCCATTGAATGGTTTTAATAATAGACAAGTAGTACTAGCGTA
TGCGAATCCAACACTATCTGCGATATAAtaagTCGACCTGCAGCTAATGTATTAGTTAAATATTAAAACTTACCACG
TAAAACTTAAAATTTAAAATGATATTTCATTGACAGATAGATCACACATTATGAACTTTCAAGGACTTGTGTTAACT
GACAATTGCAAAAATCAATGGGTCGTTGGACCATTAATAGGAAAAGGTGGATTTGGTAGTATTTATACTACTAATGA
CAATAATTATGTAGTAAAAATAGAGCCCAAAGCTAACGGATCATTATTTACCGAACAGGCATTTTATACTAGAGTAC
TTAAACCATCCGTTATCGAAGAATGGAAAAAATCTCACAATATAAAGCACGTAGGTCTTATCACGTGCAAGGCATTT
GGTCTATACAAATCCATTAATGTGGAATATCGATTCTTGGTAATTAATAGATTAGGTGCAGATCTAGATGCGGTGAT
CAGAGCCAATAATAATAGATTACCAAAAAGGTCGGTGATGTTGATCGGAATCGAAATCTTAAATACCATACAATTTA
TGCACGAGCAAGGATATTCTCACGGAGATATTAAAGCGAGTAATATAGTCTTGGATCAAATAGATAAGAATAAATTA
TATCTAGTGGATTACGGATTGGTTTCTAAATTCATGTCAAGCTTGTCTCCCTATAGTGAGTCGTATTAGAGCTTGGC
GTAATCATGGTCATAGCTGTTTCCTGTGTGAAATTGTTATCCGCTCACAATTCCACACAACATACGAGCCGGAAGCA
TAAAGTGTAAAGCCTGGGGTGCCTAATGAGTGAGCTAACTCACATTAATTGCGTTGCGCTCACTGCCCGCTTTCGAG
TCGGGAAACCTGTCGTGCCAGCTGCATTAATGAATCGGCCAACGCGCGGGGAGAGGCGGTTTGCGTATTGGGCGCTC
TTCCGCTTCCTCGCTCACTGACTCGCTGCGCTCGGTCGTTCGGCTGCGGCGAGCGGTATCAGCTCACTCAAAGGCGG
TAATACGGTTATCCACAGAATCAGGGGATAACGCAGGAAAGAACATGTGAGCAAAAGGCCAGCAAAAGGCCAGGAAC
CGTAAAAAGGCCGCGTTGCTGGCGTTTTTCGATAGGCTCCGCCCCCCTGACGAGCATCACAAAAATCGACGCTCAAG
TCAGAGGTGGCGAAACCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTTTCTCATAGCTCACGC
TGTAGGTATCTCAGTTCGGTGTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATCGCCACTGGCAGCAGCCACTG
GTAACAGGATTAGCAGAGCGAGGTATGTAGGCGGTGCTACAGAGTTCTTGAAGTGGTGGCCTAACTACGGCTACACT
AGAAGGACAGTATTTGGTATCTGCGCTCTGCTGAAGCCAGTTACCTTCGGAAAAAGAGTTGGTAGCTCTTGATCCGG
CAAACAAACCACCGCTGGTAGCGGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACGTTAAGGGATTTTGGTCATGAGA
TTATCAAAAAGGATCTTCACCTAGATCCTTTTAAATTAAAAATGAAGTTTTAAATCAATCTAAAGTATATATGAGTA
AACTTGGTCTGACAGTTACCAATGCTTAATCAGTGAGGCACCTATCTCAGCGATCTGTCTATTTCGTTCATCCATAG
TTGCCTGACTCCCCGTCGTGTAGATAACTACGATACGGGAGGGCTTACCATCTGGCCCCAGTGCTGCAATGATACCG
CGAGACCCACGCTCACCGGCTCCAGATTTATCAGCAATAAACCAGCCAGCCGGAAGGGCCGAGCGCAGAAGTGGTCC
TGCAACTTTATCCGCCTCCATCCAGTCTATTAATTGTTGCCGGGAAGCTAGAGTAAGTAGTTCGCCAGTTAATAGTT
TGCGCAACGTTGTTGGCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCACTGCATAATTCTCTTACTGTCATGCCAT
CCGTAAGATGCTTTTCTGTGACTGGTGAGTACTCAACCAAGTCATTCTGAGAATAGTGTATGCGGCGACCGAGTTGC
TCTTGCCCGGCGTCAATACGGGATAATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGTAACCCACTCGTGCACCCAACTGAT
CTTCAGCATCTTTTACTTTCACCAGCGTTTCTGGGTGAGCAAAAACAGGAAGGCAAAATGCCGCAAAAAAGGGAATA
AGGGCGACACGGAAATGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACATTTCCCCGAAAAGTGC
CACCTGACGTCTAAGAAACCATTATTATCATGACATTAACCTATAAAAATAGGCGTATCACGAGGCCCTTTCGTCTC
GCGCGTTTCGGTGATGACGGTGAAAACCTCTGACACATGCAGCTCCCGGAGACGGTCACAGCTTGTCTGTAAGCGGA
TGCCGGGAGCAGACAAGCCCGTCAGGGCGCGTCAGCGGGTGTTGGCGGGTGTCGGGGCTGGCTTAACTATGCGGCAT
CAGAGCAGATTGTACTGAGAGTGCACCATATGCGGTGTGAAATACCGCACAGATGCGTAAGGAGAAAATACCGCATC
AGGCGCCATTCGCCATTCAGGCTGCGCAACTGTTGGGAAGGGCGATCGGTGCGGGCCTCTTCGCTATTACGCCAGCT
GGCGAAAGGGGGATGTGCTGCAAGGCGATTAAGTTGGGTAACGCCAGGGTTTTCCCAGTCACGACGTTGTAAAACGA
CGGCCAGTGAATTGGATTTAGGTGACACTATA

TABLE 10
Marburg Glycoprotein mutation table

| Changes | Mutation position |
|---|---|
| (Silent mutation) | on GP |
| T to C | 18 |
| T to C | 21 |
| A to G | 129 |
| A to G | 174 |
| A to G | 237 |
| T to C | 429 |
| T to C | 480 |
| A to G | 516 |
| A to G | 666 |
| A to G | 861 |
| A to G | 1125 |
| A to G | 1143 |
| A to G | 1182 |
| A to G | 666 |
| A to G | 861 |
| A to G | 1125 |
| A to G | 1143 |
| A to G | 1182 |
| A to G | 1302 |
| A to G | 1395 |
| T to C | 1404 |
| A to G | 1527 |
| T to C | 1605 |
| T to C | 1608 |
| A to G | 1650 |
| A to G | 1656 |
| T to C | 1749 |
| A to G | 1884 |
| A to G | 1899 |
| T to C | 2028 |
| A to G | 2034 |

*Shown as lower case in SEQ ID NO: 15

SEQ ID 15:
pGEO-MARV2014 GP sequence optimized for insertion into MVA vector: (FIG. 10)
GAATTCCCTGGGACATACGTATATTTCTATGATCTGTCTTATATGAAGTCTATACAGCGAATAGATTCAGAATTTC
TACATAATTATATATTGTACGCTAATAAGTTTAATCTAACACTCCCCGAAGATTTGTTTATAATCCCTACAAATTT
GGATATTCTATGGCGTACAAAGGAATATATAGACTCGTTCGATATTAGTACAGAAACATGGAATAAATTATTATCC
AATTATTATATGAAGATGATAGAGTATGCTAAACTTTATGTACTAAGTCCTATTCTCGCTGAGGAGTTGGATAATT
TTGAGAGGACGGGAGAATTAACTAGTATTGTACAAGAAGCCATTTTATCTCTAAATTTACGAATTAAGATTTTAAA
TTTTAAACATAAAGATGATGATACGTATATACACTTTTGTAAAATATTATTCGGTGTCTATAACGGAACAAACGCT
ACTATATATTATCATAGACCTCTAACGGGATATATGAATATGATTTCAGATACTATATTTGTTCCTGTAGATAATA
ACTAAGGCGCGCCTTTCATTTTGTTTTTTTCTATGCTATAAATGGTGAGCAAGGGCGAGGAGCTGTTCACCGGGGT
GGTGCCCATCCTGGTCGAGCTGGACGGCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCTGCCCGTGCCCTGGCCCACCCTCGTGA
CCACCCTGACCTACGGCGTGCAGTGCTTCAGCCGCTACCCCGACCACATGAAGCAGCACGACTTCTTCAAGTCCGC
CATGCCCGAAGGCTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAACGGCATCAAGGT
GAACTTCAAGATCCGCCACAACATCGAGGACGGCAGCGTGCAGCTCGCCGACCACTACCAGCAGAACACCCCCATC
GGCGACGGCCCCGTGCTGCTGCCCGACAACCACTACCTGAGCACCCAGTCCGCCCTGAGCAAAGACCCCAACGAGA
AGCGCGATCACATGGTCCTGCTGGAGTTCGTGACCGCCGCCGGGATCACTCTCGGCATGCACGAGCTGTACAAGTA
AGAGCTCGAGGACGGGAGAATTAACTAGTATTGTACAAGAAGCCATTTTATCTCTAAATTTACGAATTAAGATTTT
AAATTTTAAACATAAAGATGATGATACGTATATACACTTTTGTAAAATATTATTCGGTGTCTATAACGGAACAAAC
GCTACTATATATTATCATAGACCTCTAACGGGATATATGAATATGATTTCAGATACTATATTTGTTCCTGTAGATA
ATAACTAACTCGAGGCCGCTGGTACCCAACCTAAAAATTGAAAATAAATACAAAGGTTCTTGAGGGTTGTGTTAAA
TTGAAAGCGAGAAATAATCATAAATAAGCCCgggATGTGGACAACATGTTTcTTcATTTCTCTAATTCTAATTCAA
GGAATTAAAACACTACCAATTCTAGAAATTGCGTCTAATGATCAACCACAAAATGTAGATTCTGTATGTTCTGGAA
CACTACAAAAgACTGAAGATGTACATTTGATGGGTTTTACACTATCTGGACAAAAgGTAGCGGATTCTCCACTAGA
AGCGTCTAAAAGATGGGCGTTTAGAACAGGTGTACCACCAAAgAATGTTGAATATACAGAAGGAGAAGAAGCGAAA
ACTTGTTATAATATTTCTGTAACAGATCCATCTGGAAAATCTCTACTACTAGATCCACCAACTAATGTTAGAGATT
ATCCAAAATGTAAAACAATTCATCATATTCAAGGACAAAATCCACATGCGCAAGGAATTGCGCTACATCTATGGGG
AGCATTcTTTCTATATGATAGAATAGCGTCTACAACAATGTATAGAGGAAAAGTTTTcACTGAAGGAAATATTGCG
GCTATGATAGTAAATAAgACAGTTCACAAAATGATATTTTCTAGACAAGGACAAGGATATAGACATATGAATCTAA
CATCTACAAATAAATATTGGACATCTTCTAATGGAACACAAACAAATGATACAGGATGTTTTGGAACATTGCAAGA
ATATAATAGTACAAAgAATCAAACATGTGCGCCATCTAAAACTCCACCACCACCTCCAACAGCGCATCCAGAAATT
AAACCTACATCTACACCAACAGATGCGACAAGATTGAATACAACAAATCCAAATTCTGATGATGAAGATCTAACAA
CATCTGGATCTGGAAGTGGAGAACAAGAACCATATACAACAAGTGATGCGGTTACAAAgCAAGGACTATCTTCTAC
AATGCCACCAACACTATCTCCACAACCTGGAACTCCACAACAAGGTGGAAATAATACAAATCATTCTCAAGATGCG
GCGACAGAACTAGATAATACTAATACAACTGCGCAACCACCAATGCCATCTCATAATACTACAACTATTTCTACTA
ATAATACTTCTAAACATAATCTATCTACATTGTCTGAACCACCTCAAAATACTACTAATCCTAATACTCAATCTAT
GGCGACTGAAAATGAAAAgACTTCTGCGCCTCCAAAgACAACTCTACCACCAACTGAATCTCCAACAACAGAAAAg
AGTACAAATAATACAAAATCTCCAACTACAATGGAACCTAATACAACTAATGGACACTTTACATCTCCATCTTCTA
CTCCTAATTCTACAACACAACATTTGATATACTTTAGAAGAAAgAGATCTATTTTGTGGAGAGAAGGAGATATGTT
TCCATTTCTAGATGGATTGATTAATGCGCCAATTGATTTTGATCCAGTACCAAATACAAAgACAATTTTcGATGAA
TCTTCTTCTTCTGGTGCTTCTGCGGAAGAAGATCAACATGCGTCTAGTAATATTAGTCTAACATTGTCTTATCTAC
CTCATACTTCTGAAAATACTGCGTATAGTGGAGAAAATGAgAATGATTGTGATGCGGAACTAAGAATTTGGAGTGT
ACAAGAAGATGATCTAGCGGCGGGATTGTCTTGGATTCCTTTcTTcGGACCTGGAATTGAAGGACTATATACAGCG
GGATTGATTAAgAATCAgAATAATCTAGTATGTAGACTAAGAAGATTGGCGAATCAAACAGCGAAATCTCTAGAAC
TACTACTAAGAGTAACAACTGAAGAAAGAACATTcTCTTTGATTAATAGACATGCGATTGATTTTCTATTGACAAG
ATGGGGAGGAACATGTAAAGTACTAGGACCAGATTGTTGTATTGGAATAGAAGATCTATCTAGAAATATTTCAGAA
CAAATTGATCAAATTAAgAAAGATGAACAAAAgGAAGGAACTGGATGGGGACTAGGTGGAAAATGGTGGACATCTG
ATTGGGGAGTACTAACAAATCTAGGAATTCTACTATTGCTATCTATTGCGGTACTAATTGCGTTGTCTTGTATATG
TAGAATTTTcACAAAgTATATTGGATAATAAgTCGACCTGCAGTCAAACTCTAATGACCACATCTTTTTTTAGAGA
TGAAAAATTTTCCACATCTCCTTTTGTAGACACGACTAAACATTTTGCAGAAAAAAGTTTATTAGTGTTTAGATAA
TCGTATACTTCATCAGTGTAGATAGTAAATGTGAACAGATAAAAGGTATTCTTGCTCAATAGATTGGTAAATTCCA
TAGAATATATTAATCCTTTCTTCTTGAGATCCCACATCATTTCAACCAGAGACGTTTTATCCAATGATTTACCTCG
TACTATACCACATACAAAACTAGATTTTGCAGTGACGTCGTATCTGGTATTCCTACCAAACAAAATTTTACTTTTA
GTTCTTTTAGAAAATTCTAAGGTAGAATCTCTATTTGCCAATATGTCATCTATGGAATTACCACTAGCAAAAAATG
ATAGAAATATATATTGATACATCGCAGCTGGTTTTGATCTACTATACTTTAAAAACGAATCAGATTCCATAATTGC
CTGTATATCATCAGCTGAAAAACTATGTTTTACACGTATTCCTTCGGCATTTCTTTTTAATGATATATCTTGTTTA
GACAATGATAAAGTTATCATGTCCATGAGAGACGCGTCTCCGTATCGTATAAATATTTCATTAGATGTTAGACGCT
TCATTAGGGGTATACTTCTATAAGGTTTCTTAATCAGTCCATCATTGGTTGCGTCAAGAACAAGCTTGTCTCCCTA
TAGTGAGTCGTATTAGAGCTTGGCGTAATCATGGTCATAGCTGTTTCCTGTGTGAAATTGTTATCCGCTCACAATT
CCACACAACATACGAGCCGGAAGCATAAAGTGTAAAGCCTGGGGTGCCTAATGAGTGAGCTAACTCACATTAATTG
CGTTGCGCTCACTGCCCGCTTTCGAGTCGGGAAACCTGTCGTGCCAGCTGCATTAATGAATCGGCCAACGCGCGGG
GAGAGGCGGTTTGCGTATTGGGCGCTCTTCCGCTTCCTCGCTCACTGACTCGCTGCGCTCGGTCGTTCGGCTGCGG
CGAGCGGTATCAGCTCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCGATAGGCTCCGCCCCCC
TGACGAGCATCACAAAAATCGACGCTCAAGTCAGAGGTGGCGAAACCCGACAGGACTATAAAGATACCAGGCGTTT
CCCCCTGGAAGCTCCCTCGTGCGCTCTCCTGTTCCGACCCTGCCGCTTACCGGATACCTGTCCGCCTTTCTCCCTT
CGGGAAGCGTGGCGCTTTCTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAGGTATGTAGGCGGTGCTACAG
AGTTCTTGAAGTGGTGGCCTAACTACGGCTACACTAGAAGGACAGTATTTGGTATCTGCGCTCTGCTGAAGCCAGT
TACCTTCGGAAAAAGAGTTGGTAGCTCTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGATCTTTTCTACGGGGTCTGACGCTCAGT
GGAACGAAAACTCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGTGTAGATAACTACGATAC
GGGAGGGCTTACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGTTGTTGGCATTGCTACAGGCATCG
TGGTGTCACGCTCGTCGTTTGGTATGGCTTCATTCAGCTCCGGTTCCCAACGATCAAGGCGAGTTACATGATCCCC
CATGTTGTGCAAAAAAGCGGTTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCAATACGGGATAATAC
CGCGCCACATAGCAGAACTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATCTTTTACTTTCACCAGCG
TTTCTGGGTGAGCAAAAACAGGAAGGCAAAATGCCGCAAAAAAGGGAATAAGGGCGACACGGAAATGTTGAATACT
CATACTCTTCCTTTTTCAATATTATTGAAGCATTTATCAGGGTTATTGTCTCATGAGCGGATACATATTTGAATGT
ATTTAGAAAAATAAACAAATAGGGGTTCCGCGCACATTTCCCCGAAAAGTGCCACCTGACGTCTAAGAAACCATTA
TTATCATGACATTAACCTATAAAAATAGGCGTATCACGAGGCCCTTTCGTCTCGCGCGTTTCGGTGATGACGGTGA
AAACCTCTGACACATGCAGCTCCCGGAGACGGTCACAGCTTGTCTGTAAGCGGATGCCGGGAGCAGACAAGCCCGT
CAGGGCGCGTCAGCGGGTGTTGGCGGGTGTCGGGGCTGGCTTAACTATGCGGCATCAGAGCAGATTGTACTGAGAG
TGCACCATATGCGGTGTGAAATACCGCACAGATGCGTAAGGAGAAAATACCGCATCAGGCGCCATTCGCCATTCAG
GCTGCGCAACTGTTGGGAAGGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACGACGGCCAGTGAATTGGATT
TAGGTGACACTATA

### Example 5: MVA Vaccine Incorporating Lassa Virus Sequences

In an exemplary embodiment, sequences from Lassa Virus (LARV) are prepared and optimized in shuttle plasmids and then the viral sequences are incorporated into an MVA vector. Such MVA vectors may be used individually as part of an administration protocol to elicit an immune response to Lassa Virus or as part of a multivalent vaccine composition having one or more MVA vectors expressing EBOV and Lassa Virus antigens to elicit an immune response. Original Lassa GP and Z Sequences are obtained from Genbank (GenBank: JN650517.1 and JN650518.1) and optimized as described herein for insertion into MVA vectors.

TABLE 11
Lassa Glycoprotein mutation table

| Changes | Mutation position |
|---|---|
| (Silent mutation) | on GP |
| T to C | 21 |
| T to C | 24 |
| T to C | 114 |
| A to G | 264 |
| T to C | 351 |
| A to G | 375 |
| A to G | 378 |
| A to G | 483 |
| T to C | 573 |
| A to G | 669 |
| T to C | 699 |
| T to C | 786 |
| A to G | 816 |
| A to G | 912 |
| A to G | 1056 |
| T to C | 1197 |
| A to G | 1251 |
| A to G | 1275 |
| T to C | 1308 |
| T to C | 1320 |
| A to G | 1353 |

*Shown as lower case in SEQ ID NO: 16

Exemplary Lassa Virus GP and Z sequences are provided below.

SEQ ID 16:
Optimized Lassa GP sequence (GVX-LAS.GP) for
insertion into MVA vector:
ATGGGACAAATAGTAACATTcTTcCAAGAAGTACCACATGTAATTGAAGA
AGTAATGAATATTGTACTAATTGCGCTATCTGTACTAGCGGTATTGAAAG
GATTGTATAATTTcGCGACATGTGGACTAGTAGGACTAGTTACATTTCTA
CTACTATGTGGAAGATCTTGTACAACTTCTTTGTATAAAGGAGTATATGA
ACTACAAACACTAGAATTGAATATGGAAACTCTAAATATGACAATGCCTC
TATCATGTACAAAgAATAATTCTCATCATTATATTATGGTTGGAAATGAA
ACAGGACTAGAACTAACACTAACAAATACTTCTATTATTAATCATAAATT
cTGTAATCTATCTGATGCGCATAAgAAgAATCTATATGATCATGCGCTAA
TGTCTATTATTTCTACATTTCATCTATCTATTCCAAACTTTAATCAATAT
GAAGCTATGTCTTGTGACTTTAATGGTGGAAAgATTTCTGTACAATATAA
TCTAAGTCATTCTTATGCGGGAGATGCGGCGAATCATTGTGGAACAGTAG
CGAATGGTGTACTACAAACTTTcATGAGAATGGCGTGGGGAGGATCTTAT
ATTGCGCTAGATTCTGGAAGAGGAAATTGGGATTGTATTATGACATCTTA
TCAATATCTAATTATTCAgAATACAACATGGGAAGATCATTGTCAATTcT
CTAGACCATCTCCAATAGGATATCTAGGACTACTATCTCAAAGAACAAGA
GATATATATATTAGTAGAAGATTGCTAGGAACTTTcACATGGACACTATC
TGATTCTGAAGGAAAgGATACACCTGGAGGATATTGTCTAACAAGATGGA
TGCTAATTGAAGCGGAATTGAAATGTTTTGGAAATACTGCGGTAGCGAAA
TGTAATGAAAAgCATGATGAAGAATTTTGTGATATGCTAAGACTATTTGA
CTTTAATAAACAAGCGATTCAAAGATTGAAAGCGGAAGCGCAAATGAGTA
TTCAATTGATAAATAAAGCGGTTAATGCTTTGATTAATGATCAACTAATT
ATGAAgAATCATCTAAGAGATATTATGGGAATTCCATATTGTAATTATAG
TAAATATTGGTATCTAAATCATACAACAACTGGAAGAACATCTCTACCAA
AATGTTGGCTAGTATCTAATGGATCTTATCTAAATGAAACACATTTcTCT
GATGATATTGAACAACAAGCGGATAATATGATTACAGAAATGCTACAAAA
gGAATATATGGAAAGACAAGGAAAgACACCACTAGGATTGGTAGATCTAT
TTGTTTTcTCTACATCTTTcTATCTAATTAGTATATTTCTACATCTAGTA
AAgATTCCAACACATAGACATATAGTAGGAAAATCTTGTCCAAAACCACA
TAGATTGAATCATATGGGAATATGTTCTTGTGGATTGTATAAACAACCAG
GTGTACCAGTTAAATGGAAAAGATAAtaa
SEQ ID 17:
Optimized Z sequence (GVX-LAS.Z) for insertion
into MVA vector:
ATGGGAAATAAACAAGCGAAAGCGCCAGAATCTAAAGATTCTCCAAGAGC
GAGTCTAATTCCAGATGCGACACATCTAGGACCACAATTTTGTAAATCTT
GTTGGTTTGAAAATAAAGGACTAGTAGAATGTAATAATCATTATCTATGT
CTAAATTGTCTAACACTACTACTATCTGTATCTAATAGATGTCCAATATG
CAAAATGCCACTACCAACAAAACTAAGACCATCTGCTGCTCCAACAGCGC
CACCAACAGGTGCTGCTGATTCTATTAGACCACCACCATATTCTCCATAA
taa

### Example 6: Immunogenic and Protective Potential of the MVA/Z-VLP Vaccine

To test for the immunogenic and protective potential of the MVA/Z-VLP vaccine, two rodent models for Ebola virus (EBOV) infection and disease were tested for vaccine-elicited immune responses and protection against an EBOV challenge. The guinea pig and Syrian Golden Hamster (SGH) models were chosen because of the extensive experience with these models and the availability of suitable challenge stocks at the NIH Rocky Mountain Laboratories (RML) where challenges can be conducted under BSL4 containment.

### Animal Study

Hamsters and guinea pigs were acquired by BIOQUAL, Inc., and randomized into two groups per species: a six-animal MVA/Z-VLP group and a two-animal MVA control (parental MVA, with no vaccine insert) group. Animals in the MVA/Z-VLP and MVA control groups were immunized intramuscularly at BIOQUAL. Two groups of naïve animals were also acquired and housed at BIOQUAL but were not vaccinated. All animals (MVA/Z-VLP, MVA control, and naïve control) were shipped to RML for challenge of the guinea pigs with guinea pig-adapted and the hamsters with mouse-adapted EBOV. Challenge was intraperitoneal with 10 plaque forming units of the respective adapted EBOV strains.

Table 12 summarizes the trial groups and procedures.

TABLE 12
Trial Groups and Procedures
Trial Groups and Procedures

| | | | Immunization and | | | Sampling | |
|---|---|---|---|---|---|---|---|
| | | | bleed schedule | | | schedule (days | |
| | Species | | (week in study) | | | post challenge)³ | |
| | and no. of | | | Bleed for | | | Bleed for |
| Group | animals¹ | Vaccine² | Imm. | serum | Challenge | Weight | serum |
| 1 | 6 guinea | MVA/Z-VLP | 0, 4 | 0, 4, 6 | 11 | 1-14 | 42 |
| | pigs⁴ | | | | | | |
| 2 | 2 guinea | Parental MVA | 0, 4 | 0, 4, 6 | 11 | 1-14 | 42 |
| | pigs | | | | | | |
| 3 | 6 guinea | none | N/A | 0, 4, 6 | 11 | 1-14 | 42 |
| | pigs | | | | | | |
| 4 | 6 SGH | MVA/Z-VLP | 0, 4 | 0, 4, 6 | 11 | 1-14 | 42 |
| 5 | 2 SGH | Parental MVA | 0, 4 | 0, 4, 6 | 11 | 1-14 | 42 |
| 6 | 6 SGH | none | N/A | 0, 4, 6 | 11 | 1-14 | 42 |

¹Young adult animals were used for vaccinations
²MVA/Z-VLP and parental MVA were used at a dose of 1 × 10⁸ tissue culture infectious doses (TCID)50
³Animals were euthanized on day 42.
⁴One guinea pig died of unrelated causes before the 2^{nd} vaccination Immune Responses

Vaccine induced binding Ab was determined by an ELISA using a secreted EBOV glycoprotein produced by a recombinant baculovirus in insect cells Plates were coated with the secreted EBOV glycoprotein or a control baculovirus supernatant that expressed no EBOV antigens. After blocking with 5% dry milk in 2% normal goat serum, serial serum dilutions were added to duplicate wells coated with both the EBOV glycoprotein and control supernatant. Antibody binding was detected by peroxidase-labeled anti-guinea pig IgG or peroxidase-labeled anti-hamster IgG and tetramethylbenzidine substrate. Reactions were stopped with 1N hydrochloric acid. Each plate included a standard curve generated using anti-guinea pig IgG and guinea pig IgG or anti-hamster IgG and hamster IgG. Standard curves were fitted, and sample concentrations were interpolated as micrograms of antibody per milliliter of serum using SoftMax Pro v.5.4.5. Background was calculated as antibody raised in wells coated with control baculovirus supernatant and was subtracted from EBOV glycoprotein antibody titers to obtain final results. These data are shown in FIGS. 11A and 11B.

The results of the binding Ab assays showed a single inoculation of MVA/Z-VLP eliciting similar titers of binding Ab as a single inoculation of a chimeric VSV expressing GP. It was, a chimeric VSV vector (rVSV-ZEBOV), which achieved protection against Ebola in Guinea (Agnandj S. T., N Engl J Med (2015)).

### Neutralizing Antibody

Neutralizing antibody titers were determined by focus reduction neutralization assay. Vero cells were seeded into 96-well plates at a density adequate to generate a confluent monolayer on the day of infection. Serum dilutions were prepared in PBS. For each dilution, 10 μL of diluted serum was mixed with 10 μL of medium containing 100 focus-forming units (PFU) of ZEBOV-GFP (total volume of 20 μL). After 30 min at 37° C., the media was removed from cells, the serum-virus mixture was added and the samples were incubated for 60 min at 37° C. Then the mixture was removed from the cells, 100 μL of 1.2% carboxymethylcellulose-MEM was added and the cells were incubated for 4 days at 37° C. The neutralizing antibody titer of the serum samples was considered positive at a dilution showing a >80% reduction in GFP-foci compared with the control without serum. These data are shown in FIGS. 12A and 12B.

The titers are comparable to those from other vaccines that have shown protective efficacy against EBOV in rodents and non-human primates. For example, neutralizing titers elicited by other EBOV vaccine candidates (including VSVΔG/ZEBOVGP, Adeno, and VLP) in rodents or non-human primates vary from 1:20 to 1:160 (Ye, L., et al. Virology 351, 260-270 (2006); Marzi, A., et al. J Infect Dis 204 Suppl 3, S1066-1074 (2011); Feldmann, H., et al. PLoS Pathog 3(2007), Warfield, K. L., et al. J Infect Dis 15, 8 (2007)).

### Challenge Results

The guinea pigs and hamsters were challenged intraperitoneally with 10 pfu of either guinea pig-adapted or mouse-adapted EBOV, respectively. The animals were weighed daily for 14 days. On day 42 post challenge, a terminal serum sample will be taken from all the survivors. These data are down in FIG. 13A-13D.

All of the control guinea pigs (the two vaccinated with parental MVA and the 6 unvaccinated) succumbed to the lethal challenge. One of the two hamsters receiving parental MVA succumbed and four of the unvaccinated SGHs succumbed. Minimal weight loss (1-2%) occurred on days 5-7 for the vaccinated guinea pigs and no weight loss, but a leveling in weight gain, occurred on days 4-6 for the vaccinated SGHs. In contrast, all of the unvaccinated animals underwent major losses in weight.

The complete protection elicited in rodents by MVA/Z-VLP is comparable to that seen from other vaccines that have shown protective efficacy. For example, it has been shown that a VSV-based vaccine candidate (VSVDG/ZEBOV) protects SGH and guinea pigs from lethal challenge with the Zaire strain of Ebola (Marzi, A., et al. J Infect Dis 204 Suppl 3, S1066-1074 (2011); Tsuda, Y., et al. J Infect Dis 204, 8, (2011)).

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

All references cited herein are incorporated by reference in their entirety.

1. A method of preventing an infection by a Marburg virus in a human subject at risk of exposure thereto, said method comprising administering to the human subject in need thereof a pharmaceutical composition in a prophylactically effective amount, wherein the pharmaceutical composition comprises:
a) at least one recombinant modified vaccinia ankara (MVA) vector comprising:
i) a first nucleic acid encoding a Marburg virus (MARV) glycoprotein, and
ii) a second nucleic acid sequence encoding a Marburg virus (MARV) VP40 matrix protein;
wherein both the first nucleic acid sequence and the second nucleic acid sequence are under the control of one or more promoters compatible with poxvirus expression systems;
wherein the first nucleic acid is located between MVA genes I8R and G1L;
wherein the second nucleic acid sequence is located between MVA genes A50R and B1R in a restructured and modified deletion site III; and
wherein the MARV glycoprotein and the MARV VP40 matrix protein are capable of assembling into virus-like particles (VLPs) when expressed in a host cell; and
b) a pharmaceutically acceptable carrier.

2. The method of claim 1, wherein the first nucleic acid sequence and the second nucleic acid sequence are optimized by one or more method selected from the group consisting of i) changing selected codons to other synonymous codons that are optimal for protein expression by MVA, ii) interrupting homopolymer stretches using silent mutations, and iii) interrupting transcription terminator motifs using silent mutations.

3. The method of claim 1, wherein the MARV glycoprotein and MARV VP40 matrix protein are derived from the same MARV strain.

4. The method of claim 1, wherein the first nucleic acid sequence comprises nucleic acids 1631-3676 of SEQ ID NO: 15, or a nucleic acid sequence at least 95% identical thereto.

5. The method of claim 1, wherein the first nucleic acid sequence comprises nucleic acids 1631-3676 of SEQ ID NO: 15.

6. The method of claim 1, wherein the second nucleic acid sequence comprises nucleic acids 1581-2492 of SEQ ID NO: 14, or a nucleic acid sequence at least 95% identical thereto.

7. The method of claim 1, wherein the second nucleic acid sequence comprises nucleic acids 1581-2492 of SEQ ID NO: 14.

8. The method of claim 1, wherein the first nucleic acid sequence comprises nucleic acids 1631-3676 of SEQ ID NO: 15, and the second nucleic acid sequence comprises nucleic acids 1581-2492 of SEQ ID NO:14.

9. The method of claim 1, wherein the administration is repeated at least twice.

10. A method of preventing an infection by a Marburg virus in a human subject at risk of exposure thereto, said method comprising administering to the human subject in need thereof a pharmaceutical composition in a prophylactically effective amount, wherein the pharmaceutical composition comprises:
a) at least one recombinant modified vaccinia ankara (MVA) vector comprising:
i) a first nucleic acid sequence encoding a Marburg virus glycoprotein comprising nucleic acids 1631-3676 of SEQ ID NO: 15, or a nucleic acid sequence at least 95% identical thereto, and
ii) a second nucleic acid sequence encoding a Marburg virus VP40 matrix protein comprising nucleic acids 1581-2492 of SEQ ID NO: 14, or a nucleic acid sequence at least 95% identical thereto;
wherein both the first nucleic acid sequence and the second nucleic acid sequence are under the control of one or more promoters compatible with poxvirus expression systems;
wherein the first nucleic acid is located between MVA genes I8R and G1L;
wherein the second nucleic acid sequence is located between MVA genes A50R and B1R in a restructured and modified deletion site III; and
wherein the Marburg virus glycoprotein and the Marburg virus VP40 matrix protein are capable of assembling into virus-like particles (VLPs) when expressed in a host cell; and
b) a pharmaceutically acceptable carrier.

11. The method of claim 10, wherein the administration is repeated at least twice.
